(19) United States
(12) Patent Application Publication
(10) Pub. No.: US 2024/0140980 A1
(43) Pub. Date: May 2, 2024

(54) HETEROCYCLIC INHIBITORS OF CD73 FOR TREATMENT OF DISEASE

(71) Applicant: Teon Therapeutics, Inc., Redwood City, CA (US)

(72) Inventor: Elfatih ELZEIN, Mountain House, CA (US)

(21) Appl. No.: 18/477,946

(22) Filed: Sep. 29, 2023

Related U.S. Application Data
(60) Provisional application No. 63/378,109, filed on Oct. 3, 2022.

(51) Int. Cl. C07H 19/23; A61K 45/06

(52) U.S. Cl. CPC C07H 19/23; A61K 45/06

(57) ABSTRACT

Provided are compounds which may be useful as inhibitors of CD73 for the treatment or prevention of cancer.

This application claims the benefit of priority of U.S. provisional application No. 63/378,109, filed Oct. 3, 2022, the contents of which are incorporated by reference as if written herein in their entirety.

CD73 is a 70-kD, glycosylphosphatidylinositol (GPI) anchored cell surface protein that is encoded by NT5E gene, also known as ecto-5′-nucleotidase (ecto-5′-NT), plays a crucial role in switching on adenosinergic signaling. CD73 has both enzymatic and nonenzymatic functions in cells. As a nucleotidase, CD73 catalyzes the hydrolysis of AMP into adenosine and phosphate. Notably, CD73-generated adenosine plays an important role in tumor immunoescape. In addition to its enzymatic function, CD73 is also a signal and adhesive molecule that can regulate cell interaction with extracellular matrix (ECM) components, such as laminin and fibronectin, to mediate cancer invasive and metastatic properties. Indeed, the enzymatic and nonenzymatic functions of CD73 are both involved in cancer associated process and not completely independent of each other.

CD73 has also been found to be overexpressed in many types of cancer cell lines and patient's biopsies including breast cancer, colorectal cancer, ovarian cancer, gastric cancer, and gallbladder cancer and associated with clinical characteristics, or prognosis of cancer patients. Increasing evidence has verified that CD73 is a key regulatory molecule in cancer development. In particular, due to the favorable effect on tumor-bearing mice models, although have not been investigated in clinical patients, anti-CD73 therapy has become a promising approach for the treatment of cancer patients.

The emerging roles of CD73 in tumor growth and metastasis, especially as a key immunosuppressive factor in tumor microenvironment, have presented potential opportunities to develop anti-CD73 therapy for various human cancers. In this regard, accumulating results with small molecular inhibitors, or monoclonal antibodies targeting CD73 in mice tumor models, suggest that targeted CD73 therapy is an important approach to effective control of tumor growth and metastasis.

CD73 plays multiple roles in cancer related processes. The connection between CD73 overexpression and cancer subtype, prognosis, drug response of patients has presented the potential value of CD73 that served as a detectable biomarker of individual cancer therapy. In addition, the promotive effect of CD73 on tumor growth and metastasis suggests that CD73 is a potential therapeutic target for cancer treatment. Targeting CD73 therapy with small molecule inhibitors has displayed favorable antitumor effects in mice tumor models. These observations present a good opportunity to develop anti-CD73 therapy for the treatment of certain cancer patients.

Accordingly, a need exists for new inhibitors of CD73 for use in treatment of cancers.

### SUMMARY

Provided is a compound of Formula (I):

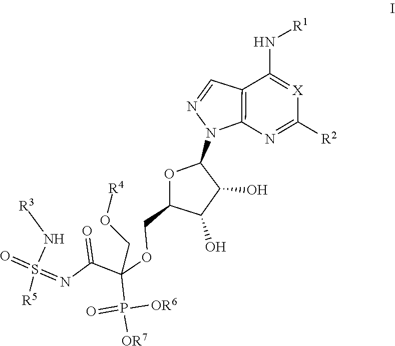

or a pharmaceutically acceptable salt thereof, wherein:
X is chosen from CH and N;
R¹ is chosen from C₁-C₆ alkyl, C₃-C₁₂ cycloalkyl, heterocycloalkyl, aryl, C₁-C₆ alkylaryl, C₁-C₆ alkylheteroaryl, C₃-C₅ cycloalkylaryl, C₃-C₅ cycloalkylheteroaryl, and heteroaryl, each of which is optionally substituted with one or more groups chosen from C₁-C₆ alkyl, C₁-C₆ alkoxy, C₁-C₆ fluoroalkyl, C₁-C₄ fluoroalkoxy, halo, and hydroxy;
R² is chosen from hydrogen, halo, aryl, cyano, C₃-C₅ cycloalkyl, and heteroaryl;
R³ is chosen from hydrogen, C₁-C₆ alkyl, —C(O)R⁸, —C(O)OR⁸, and —C(O)NHR⁸;
R⁴ is chosen from hydrogen, C₃-C₅ cycloalkyl, and C₁-C₆ alkyl;
R⁵ is C₁-C₆ alkyl;
R⁶ and R⁷ are each independently chosen from hydrogen, C₁-C₆ alkyl, —C(O)R⁹, and C₁-C₆ alkylcarboxylmethyl;
R⁸ is chosen from C₁-C₆ alkyl, C₃-C₇ cycloalkyl, 3- to 7-membered heterocycloalkyl, heteroaryl, and aryl, any of which may be substituted by one or more groups chosen from C₁-C₃ alkyl and halo; and
R⁹ is C₁-C₇ alkyl.

Also provided is a compound of Formula (I):

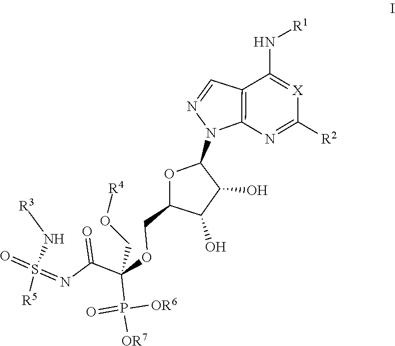

or a pharmaceutically acceptable salt thereof, wherein:
X is chosen from CH and N;
R¹ is chosen from C₁-C₆ alkyl, C₃-C₇ cycloalkyl, heterocycloalkyl, aryl, and heteroaryl, each of which is optionally substituted with C₁-C₆ alkyl, C₁-C₆ alkoxy, C₁-C₆ fluoroalkyl, C₁-C₄ fluoroalkoxy, halo, and hydroxy;
R² is chosen from hydrogen, halo, and heteroaryl;
R³ is chosen from hydrogen, C₁-C₆ alkyl, acetyl, carboxyl, C₁-C₆ alkoxycarbonyl, aminocarbonyl, and aryl optionally substituted by C₁-C₆ alkyl;
R⁴ is chosen from hydrogen and C₁-C₆ alkyl;
R⁵ is C₁-C₆ alkyl; and
R⁶ and R⁷ are each independently chosen from hydrogen and C₁-C₆ alkyl.

Also provided is a pharmaceutical composition comprising a compound or a pharmaceutically acceptable salt thereof described herein, and a pharmaceutically acceptable carrier.

Also provided is a method of treatment of a CD73-mediated disease, comprising the administration of a therapeutically effective amount of a compound or a pharmaceutically acceptable salt thereof described herein, to a patient having a CD73-mediated disease.

These and other aspects of the disclosure disclosed herein will be set forth in greater detail as the patent disclosure proceeds.

### DETAILED DESCRIPTION

### Definitions

As used in the present specification, the following words and phrases are generally intended to have the meanings as set forth below, except to the extent that the context in which they are used indicates otherwise.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “alkenyl,” as used herein, alone or in combination, refers to a straight-chain or branched-chain hydrocarbon radical having one or more double bonds and containing from 2 to 20 carbon atoms. In certain embodiments, alkenyl will comprise from 2 to 6 carbon atoms. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, alkyl will comprise from 1 to 10 carbon atoms. In further embodiments, alkyl will comprise from 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, noyl and the like.

The term “alkynyl,” as used herein, alone or in combination, refers to a straight-chain or branched chain hydrocarbon radical having one or more triple bonds and containing from 2 to 20 carbon atoms. In certain embodiments, alkynyl comprises from 2 to 6 carbon atoms. In further embodiments, alkynyl comprises from 2 to 4 carbon atoms.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “carbamate,” as used herein, alone or in combination, refers to an ester of carbamic acid (—NHCOO—) which may be attached to the parent molecular moiety from either the nitrogen or acid end.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system. In certain embodiments, cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, heteroaryl will comprise from 1 to 4 heteroatoms as ring members. In further embodiments, heteroaryl will comprise from 1 to 2 heteroatoms as ring members. In certain embodiments, heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzindolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each heteroatom may be independently chosen from nitrogen, oxygen, and sulfur. In certain embodiments, heterocycloalkyl will comprise from 1 to 4 heteroatoms as ring members. In further embodiments, heterocycloalkyl will comprise from 1 to 2 heteroatoms as ring members. In certain embodiments, heterocycloalkyl will comprise from 3 to 8 ring members in each ring. In further embodiments, heterocycloalkyl will comprise from 3 to 7 ring members in each ring. In yet further embodiments, heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like.

The term “hydroxyalkyl,” as used herein, alone or in combination, refers to a hydroxy group attached to the parent molecular moiety through an alkyl group.

The term “lower,” as used herein, alone or in a combination, where not otherwise specifically defined, means containing from 1 to and including 6 carbon atoms (i.e., C₁-C₆ alkyl).

The term “oxo,” as used herein, alone or in combination, refers to ═O.

The terms “sulfonate,” “sulfonic acid,” and “sulfonic,” as used herein, alone or in combination, refer the —SO₃H group and its anion as the sulfonic acid is used in salt formation.

The term “sulfonyl,” as used herein, alone or in combination, refers to a —S(O)₂—, —S(O)₂R, or —S(O)₂R— group, with R as defined herein.

The term “sulfonamido,” as used herein, alone or in combination, includes both N-sulfonamido and S-sulfonamido. The term “N-sulfonamido” refers to either a RS(O)₂NR′- or —S(O)₂NR′— group with R and R′ as defined herein. The term “S-sulfonamido” refers to a —S(O)₂NRR′ or —S(O)₂NR— group, with R and R′ as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “optionally substituted” means the anteceding group may be substituted or unsubstituted.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“CD73 inhibitor” is used herein to refer to a compound that exhibits an IC₅₀ with respect to CD73 activity of no more than about 100 μM and more typically not more than about 50 μM, as measured in the CD73 described generally herein. “IC₅₀” is that concentration of inhibitor which reduces the activity of an enzyme (e.g., CD73) to half-maximal level. Certain compounds disclosed herein have been discovered to exhibit inhibition against CD73.

The phrase “therapeutically effective” is intended to qualify the amount of active ingredients used in the treatment of a disease or disorder or on the effecting of a clinical endpoint.

The term “pharmaceutically acceptable” refers to those compounds (or salts) which are suitable for use in contact with the tissues of patients without undue toxicity, irritation, and allergic response, are commensurate with a reasonable benefit/risk ratio, and are effective for their intended use.

As used herein, the term “treat,” “treating,” or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The compounds disclosed herein can exist as therapeutically acceptable salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethanesulfonate (isethionate), lactate, maleate, malonate, DL-mandelate, mesitylenesulfonate, methanesulfonate, naphthalenesulfonate, nicotinate, 2-naphthalenesulfonate, oxalate, pamoate, pectinate, persulfate, 3-phenylpropionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and stearyl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

Provided is a compound of Formula (I):

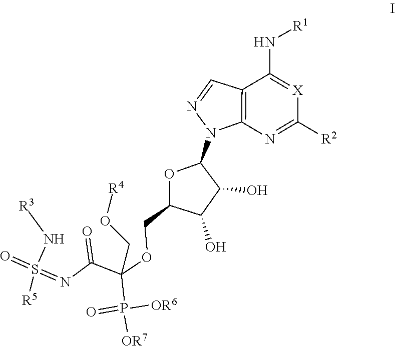

or a pharmaceutically acceptable salt thereof, wherein:
X is chosen from CH and N;
R¹ is chosen from C₁-C₆ alkyl, C₃-C₁₂ cycloalkyl, heterocycloalkyl, aryl, C₁-C₆ alkylaryl, C₁-C₆ alkylheteroaryl, C₃-C₅ cycloalkylaryl, C₃-C₅ cycloalkylheteroaryl, and heteroaryl, each of which is optionally substituted with one or more groups chosen from C₁-C₆ alkyl, C₁-C₆ alkoxy, C₁-C₆ fluoroalkyl, C₁-C₄ fluoroalkoxy, halo, and hydroxy;
R² is chosen from hydrogen, halo, aryl, cyano, C₃-C₅ cycloalkyl, and heteroaryl;
R³ is chosen from hydrogen, C₁-C₆ alkyl, —C(O)R⁸, —C(O)OR⁸, and —C(O)NHR⁸;
R⁴ is chosen from hydrogen, C₃-C₅ cycloalkyl, and C₁-C₆ alkyl;
R⁵ is C₁-C₆ alkyl;
R⁶ and R⁷ are each independently chosen from hydrogen, C₁-C₆ alkyl, —C(O)R⁹, and C₁-C₆ alkylcarboxylmethyl;
R⁸ is chosen from C₁-C₆ alkyl, C₃-C₇ cycloalkyl, 3- to 7-membered heterocycloalkyl, heteroaryl, and aryl, any of which may be substituted by one or more groups chosen from C₁-C₃ alkyl and halo; and
R⁹ is C₁-C₇ alkyl.

Also provided is a compound of Formula (I):

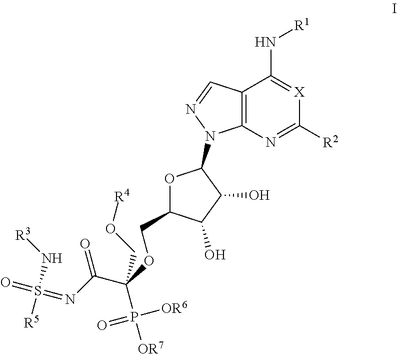

or a pharmaceutically acceptable salt thereof, wherein:
X is chosen from CH and N;
R¹ is chosen from C₁-C₆ alkyl, C₃-C₇ cycloalkyl, heterocycloalkyl, aryl, and heteroaryl, each of which is optionally substituted with C₁-C₆ alkyl, C₁-C₆ alkoxy, C₁-C₆ fluoroalkyl, C₁-C₄ fluoroalkoxy, halo, and hydroxy;
R² is chosen from hydrogen, halo, and heteroaryl;
R³ is chosen from hydrogen, C₁-C₆ alkyl, acetyl, carboxyl, C₁-C₆ alkoxycarbonyl, aminocarbonyl, and aryl optionally substituted by C₁-C₆ alkyl;
R⁴ is chosen from hydrogen and C₁-C₆ alkyl;
R⁵ is C₁-C₆ alkyl; and
R⁶ and R⁷ are each independently chosen from hydrogen and C₁-C₆ alkyl.

In some embodiments, X is N.

In some embodiments, X is CH.

In some embodiments, R¹ is cyclopentyl or cyclohexyl, each of which is optionally substituted with C₁-C₆ alkyl, C₁-C₆ alkoxy, C₁-C₆ fluoroalkyl, C₁-C₄ fluoroalkoxy, halo, and hydroxy.

In some embodiments, R¹ is cyclopentyl or cyclohexyl.

In some embodiments, R¹ is cyclopentyl.

In some embodiments, R² is halo.

In some embodiments, R² is chloro.

In some embodiments, R² is 1H-1,2,3-triazol-1-yl.

In some embodiments, X is N and R² is halo.

In some embodiments, X is N and R² is halo and R² is chloro.

In some embodiments, the compound of Formula I, or a pharmaceutically acceptable salt thereof, is a compound of Formula II,

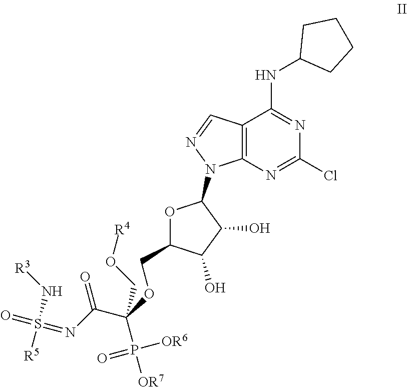

or a pharmaceutically acceptable salt thereof.

In some embodiments, the compound of Formula I, or a pharmaceutically acceptable salt thereof, is a compound of Formula III,

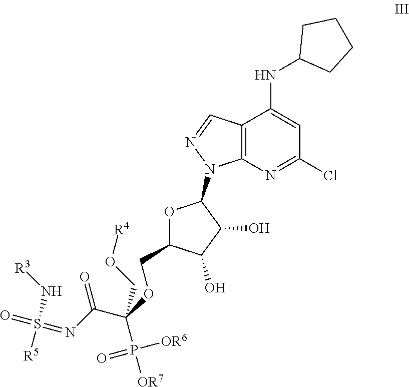

or a pharmaceutically acceptable salt thereof.

In some embodiments, R⁴ is C₁-C₆ alkyl.

In some embodiments, R⁴ is methyl.

In some embodiments, R⁶ is hydrogen.

In some embodiments, R⁷ is hydrogen.

In some embodiments, R⁶ and R⁷ are each hydrogen.

In some embodiments, R⁶ and R⁷ are each —CH₂OC(O)CH(CH₃)₂.

In some embodiments, R⁵ is methyl.

In some embodiments, R³ is chosen from hydrogen, acetyl, C₁-C₆ alkoxycarbonyl, and aminocarbonyl.

In some embodiments, R³ is chosen from hydrogen, —C(O)R⁸, —C(O)OR⁸, and —C(O)NHR⁸.

In some embodiments, R³ is hydrogen.

In some embodiments, R³ is acetyl.

In some embodiments, R³ is —C(O)OR⁸ or —C(O)NHR⁸ where R⁸ is C₁-C₃ alkyl.

In some embodiments, R³ is chosen from —C(O)OR⁸ or —C(O)NHR⁸ and R⁸ is C₁-C₃ alkyl

In some embodiments, R⁸ is methyl.

In some embodiments, R³ is —C(O)R⁸ and R⁸ is chosen from methyl, cyclopropyl, ethyl, isopropyl, oxetanyl, methylpyrazolyl, pyridinyl, and fluorophenyl.

In some embodiments, the compound of Formula I, a pharmaceutically acceptable salt thereof, is chosen from

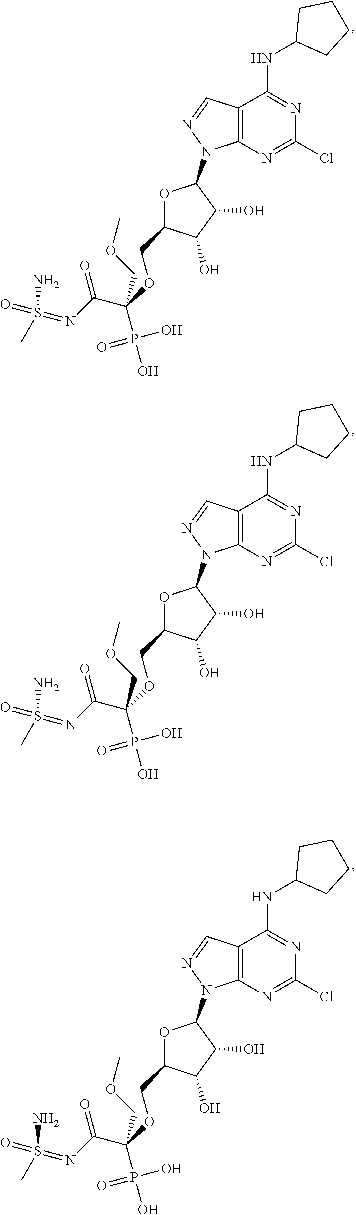 [SMILES: COC[C@@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O ; COC[C@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O]

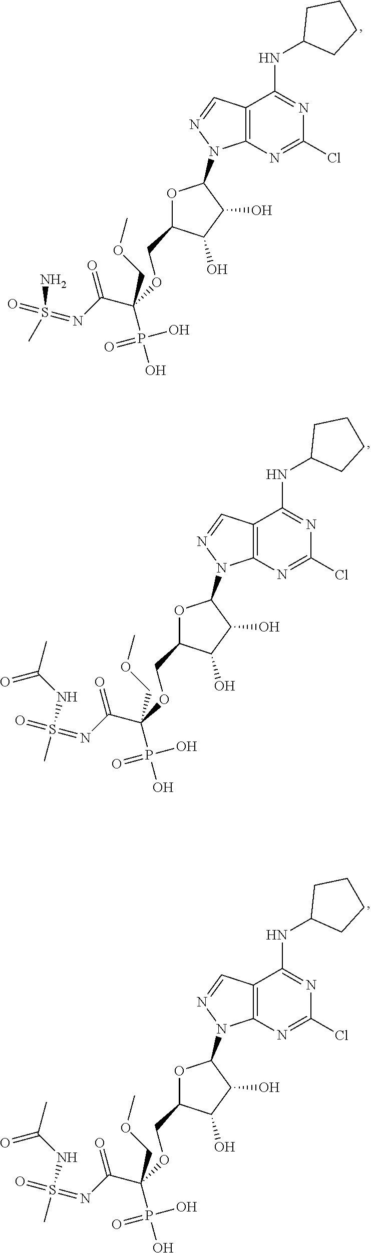 [SMILES: COC[C@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

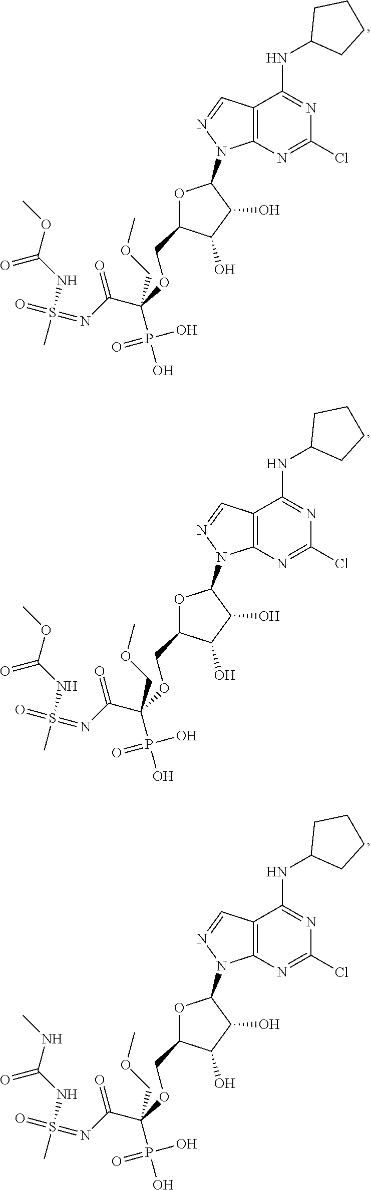 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O ; CNC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

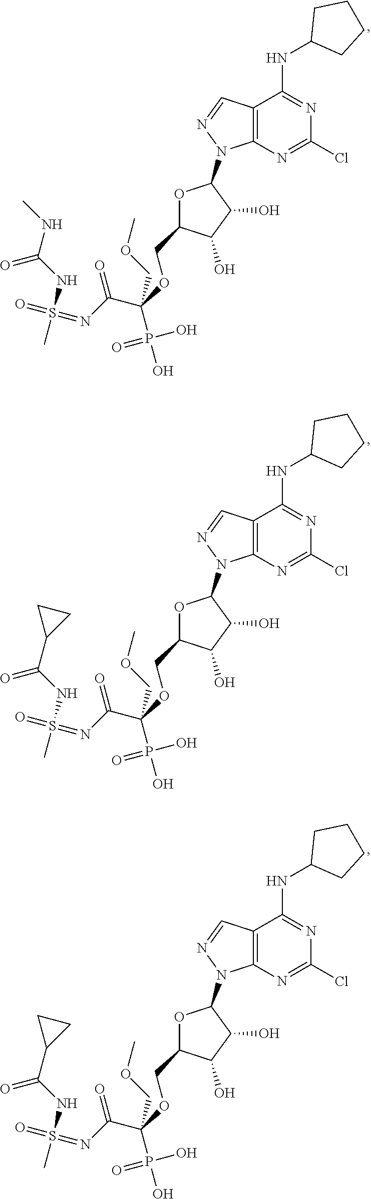 [SMILES: CNC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(O)O]

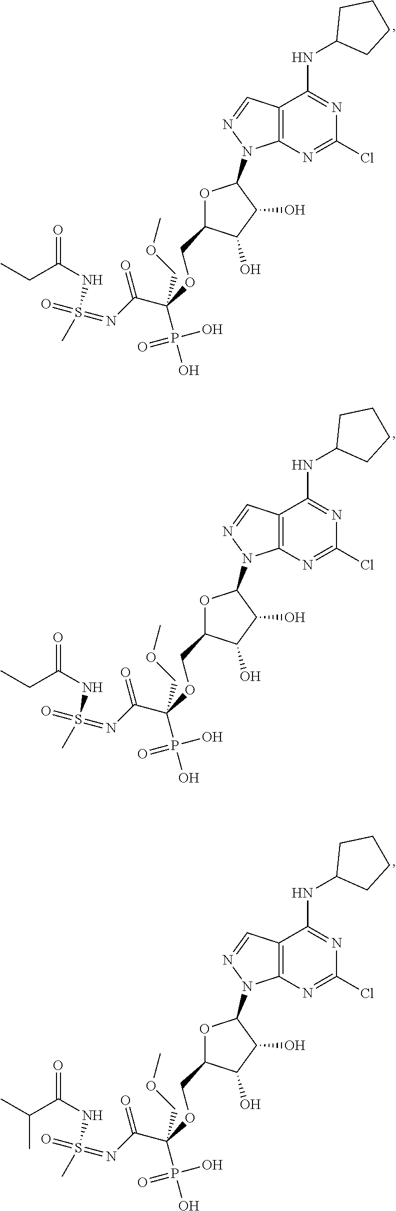 [SMILES: CCC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; CCC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C(C)C)P(=O)(O)O]

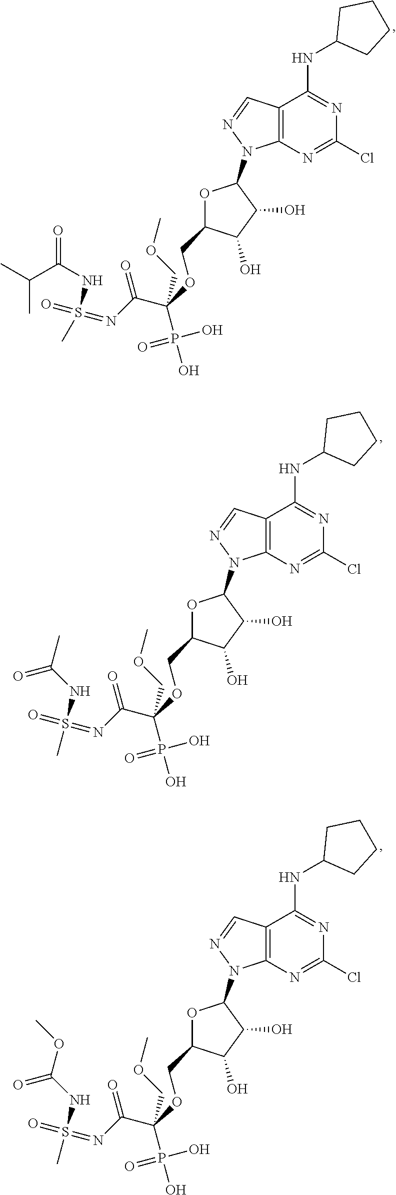 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C(C)C)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(C)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)OC)P(=O)(O)O]

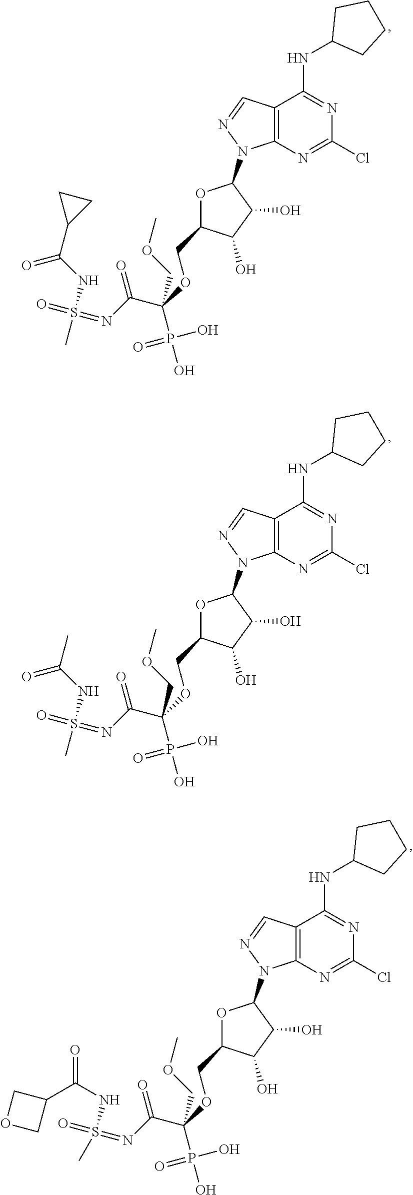 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1COC1)P(=O)(O)O]

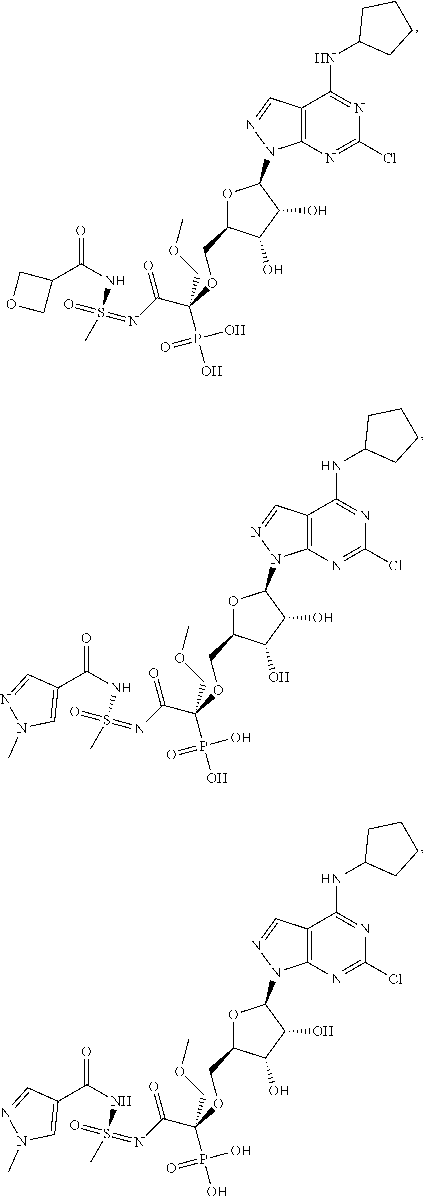 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1COC1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cnn(C)c1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1cnn(C)c1)P(=O)(O)O]

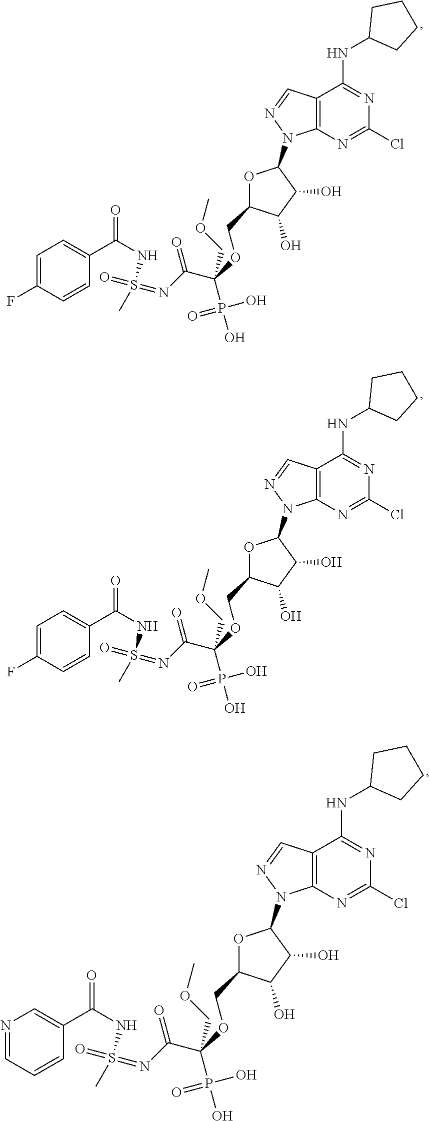 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1ccc(F)cc1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1ccc(F)cc1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cccnc1)P(=O)(O)O]

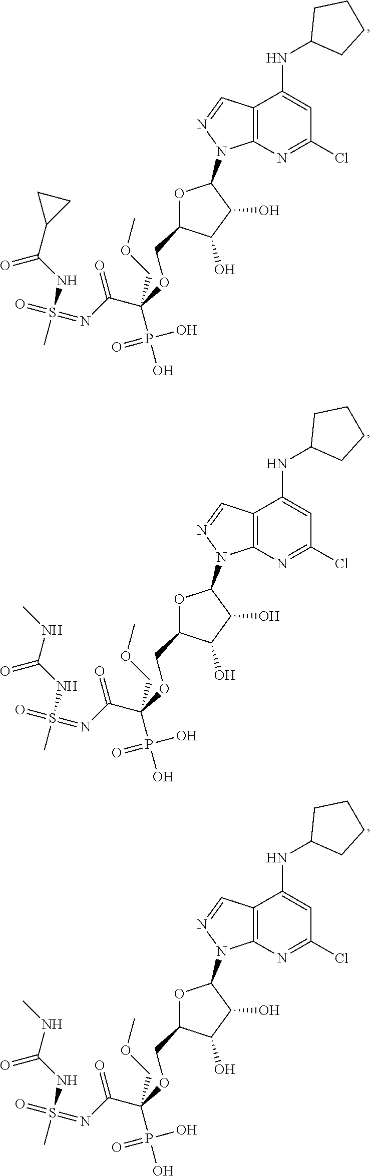 [SMILES: COC[C@@](OC[C@H]1O[C@@H](n2ncc3c(NC4CCCC4)cc(Cl)nc32)[C@H](O)[C@@H]1O)(C(=O)N=[S@](C)(=O)NC(=O)C1CC1)P(=O)(O)O ; CNC(=O)N[S@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O ; CNC(=O)N[S@@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O]

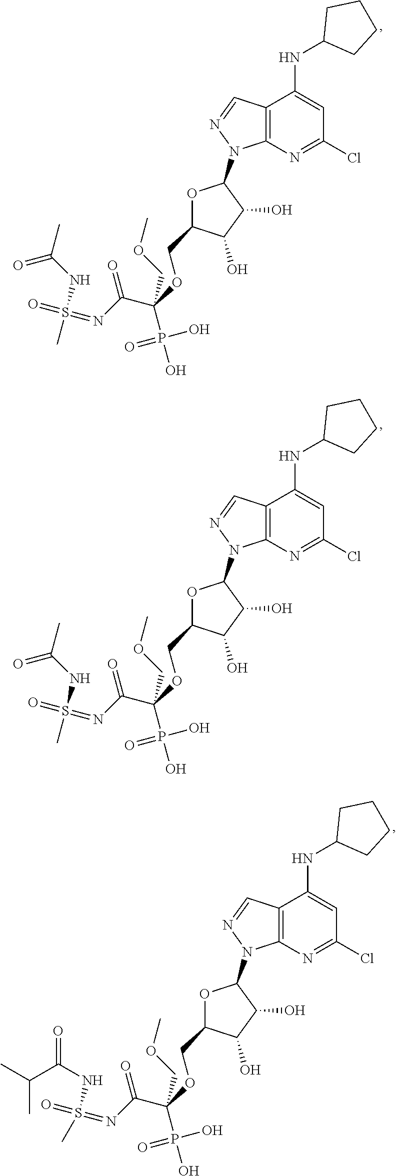 [SMILES: COC[C@@](OC[C@H]1O[C@@H](n2ncc3c(NC4CCCC4)cc(Cl)nc32)[C@H](O)[C@@H]1O)(C(=O)N=[S@@](C)(=O)NC(C)=O)P(=O)(O)O ; COC[C@@](OC[C@H]1O[C@@H](n2ncc3c(NC4CCCC4)cc(Cl)nc32)[C@H](O)[C@@H]1O)(C(=O)N=[S@](C)(=O)NC(C)=O)P(=O)(O)O ; COC[C@@](OC[C@H]1O[C@@H](n2ncc3c(NC4CCCC4)cc(Cl)nc32)[C@H](O)[C@@H]1O)(C(=O)N=[S@@](C)(=O)NC(=O)C(C)C)P(=O)(O)O]

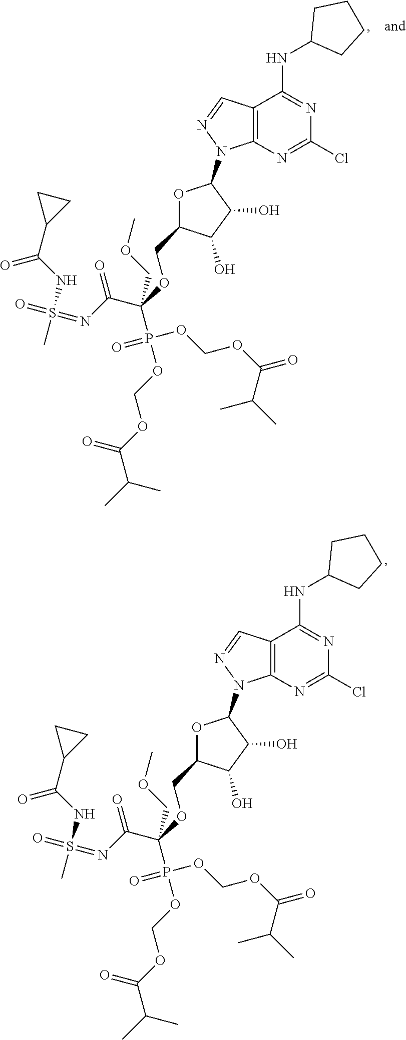 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(OCOC(=O)C(C)C)OCOC(=O)C(C)C ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(OCOC(=O)C(C)C)OCOC(=O)C(C)C]

or a Pharmaceutically acceptable salt thereof.

### Pharmaceutical Compositions

While it may be possible for the compounds described herein to be administered as the raw chemical, it is also possible to present them as a pharmaceutical formulation. Accordingly, provided herein are pharmaceutical compositions which comprise one or more of certain compounds disclosed herein, or one or more pharmaceutically acceptable salts thereof, together with one or more pharmaceutically acceptable carriers thereof and optionally one or more other therapeutic ingredients.

The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

### Indications and Methods of Treatment

Also provided is a method of treatment of a CD73-mediated disease, comprising the administration of a therapeutically-effective amount of a compound described herein, or a pharmaceutically acceptable salt thereof, to a patient having a CD-73-mediated disease.

In some embodiments, the CD73-mediated disease is cancer.

In some embodiments, the cancer is breast cancer.

In some embodiments, the breast cancer is triple-negative breast cancer.

In some embodiments, the cancer is chosen from melanoma, renal cell carcinoma, colorectal carcinoma, pancreatic cancer, prostate cancer, ovarian cancer, gastric cancer, leukemia and lymphoma.

### Combination Therapy

In certain instances, it may be appropriate to administer at least one of the compounds described herein (or a pharmaceutically acceptable salt thereof) in combination with another therapeutic agent. Accordingly, also provided is a method further comprises administering one or more additional pharmaceutically active agents; such as wherein the additional pharmaceutically active agent is a chemotherapeutic agent.

By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds or salts described herein with chemotherapeutic agents.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Further embodiments include the embodiments disclosed in the following Examples, which is not to be construed as limiting in any way.

### Synthetic Methods

### General Schemes

Schemes below provide exemplary synthetic methods for the preparation of the compounds provided herein. One of ordinary skills in the art will understand that similar methods may be employed to prepare the compounds provided herein. In other words, one of ordinary skills in the art will recognize that suitable adjustments to reagents, protecting groups, reaction conditions, reaction sequences, purification methods, and chiral separation conditions may be employed to prepare a desired embodiment. The reactions may be scaled upwards or downwards to suit the amount of material to be prepared. In some embodiment, the compound of Formula I may be prepared following the schemes provided herein, using suitable starting materials known in the art and/or available from a commercial source. In one embodiment, the starting materials of the schemes provided herein may be prepared from commercially available compounds using procedures and conditions known in the art. In some embodiments, provided herein is a process of preparing a compound of Formula I, or a pharmaceutically acceptable salt thereof.

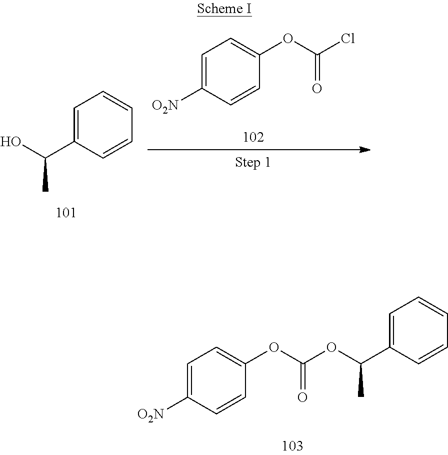

Referring to Scheme I, Step 1, to a solution of the compound of Formula 101 in an organic solvent, such as dichloromethane is added an organic base such as pyridine and the compound of Formula 102. The mixture is stirred at ambient temperature for 16 h. The product, a compound of Formula 103, is isolated and purified using methods known in the art.

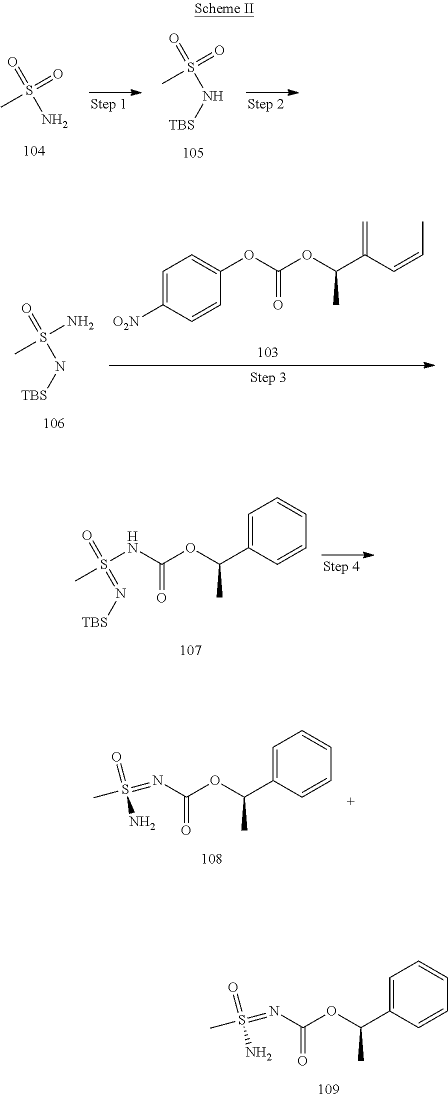

Referring to Scheme II, Step 1, to a solution of the compound of Formula 104 in an organic solvent, such as dichloromethane is added a protecting group such as tert-butyldimethylsilyl chloride and an organic base, such as triethylamine. The mixture is stirred at ambient temperature for 16 h. The product, a compound of Formula 105, is isolated and purified using methods known in the art.

Referring to Scheme II, Step 2, to a mixture of 1,1,1,2,2,2-hexachloroethane in dichloromethane was added a nucleophile such as PPh₃ and the mixture was stirred at elevated temperatures for 18 h, and then an organic base such as triethylamine was added at ambient temperature. The resulting mixture was stirred at ambient temperature for 0.5 h, and then the compound of Formula 105 was added at 0° C., the resulting mixture was stirred at 0° C. for 0.5 h, NH₃ was bubbled into the solution at 0° C. for 6 minutes, the resulting mixture was stirred at ambient temperature for 1 h. The product, a compound of Formula 106, is isolated and purified using methods known in the art.

Referring to Scheme II, Step 3, to a solution of the compound of Formula 106 in an organic solvent such as THF was added an organolithium reagent such as n-butyl lithium, the mixture was stirred at −78° C. for 0.5 h. To the mixture was added the compound of Formula 103 at −78° C., the mixture was stirred at 0° C. for 1 h. The product, a compound of Formula 107, is isolated and purified using methods known in the art.

Referring to Scheme II, Step 4, to a solution of a compound of Formula 107 in an organic solvent such as THF is added a deprotecting agent such as tetra-n-butylammonium fluoride. The mixture was stirred at ambient temperature for 1 h. The products, compounds of Formulas 107 and 108, are isolated and purified using methods known in the art. Individual enantiomers can be separated by using methods known in the art, such as chiral chromatography.

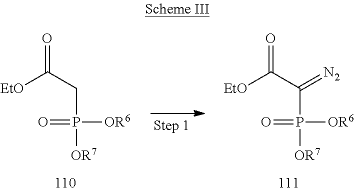

Referring to Scheme III, Step 1, to a solution of the compound of Formula 110 in an organic solvent, such as DMSO is added tosyl azide and an inorganic base, such as K₂CO₃. The mixture is stirred at ambient temperature for 1 h. The product, a compound of Formula 111, is isolated and purified using methods known in the art.

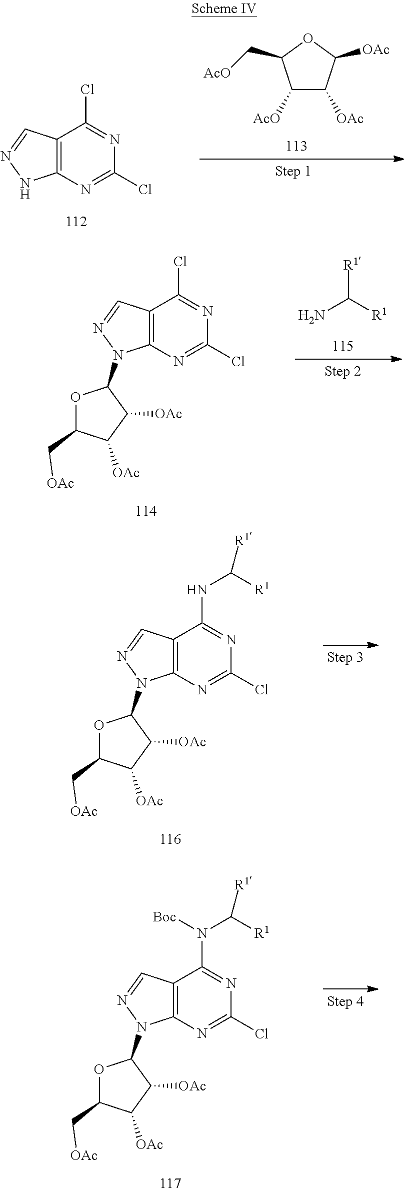

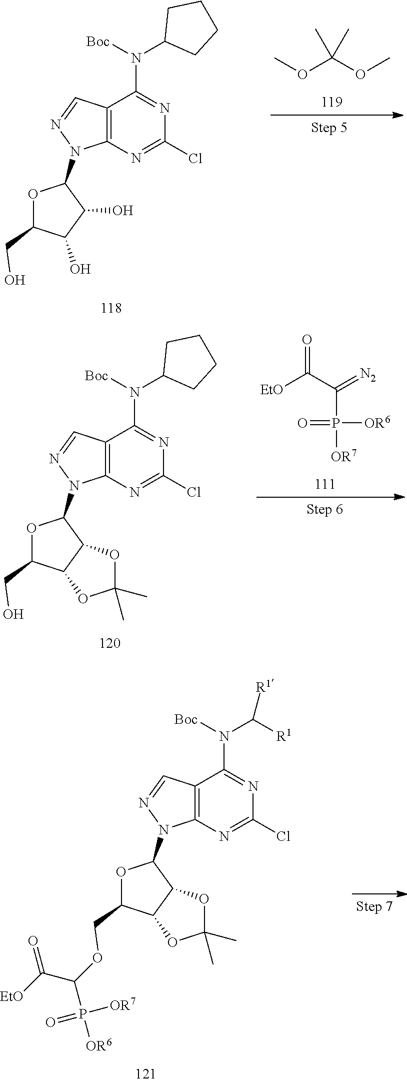

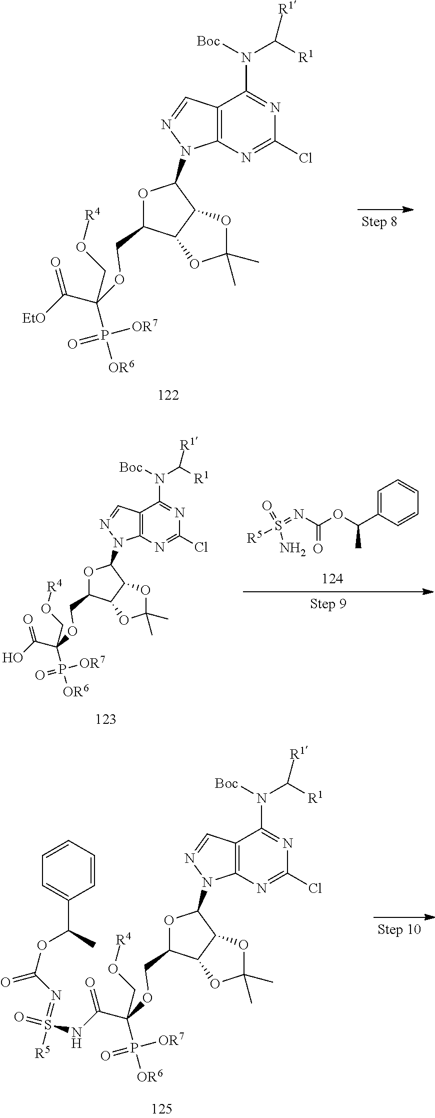

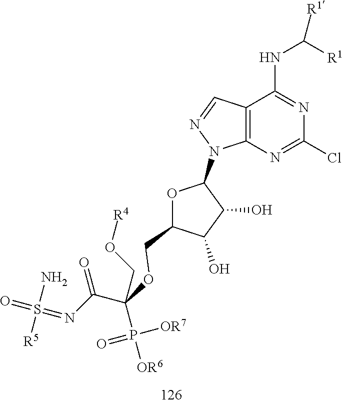

Referring to Scheme IV, Step 1, to a solution of the compound of Formula 112 in a solvent such as hexamethyldisilazane is added an inorganic salt such as ammonium sulfate. The mixture is stirred at reflux for 3 h. The mixture is then filtered and concentrated using methods known in the art. To the crude product is added a solution of the compound of Formula 113 in an organic solvent such as acetonitrile at ambient temperature. Then, trimethylsilyl trifluoromethanesulfonate is added dropwise at 0° C., and the resulting mixture is stirred at ambient temperature for 12 h. A strong base, such as sodium hydroxide, is added to the solution along with water dropwise at 0° C. to pH=8. The product, a compound of Formula 114, is isolated and purified using methods known in the art.

Referring to Scheme IV, Step 2, to a solution of a compound of Formula 114 is added an organic base, such as triethylamine and a compound of Formula 115. The mixture is stirred at elevated temperature for 1 h. The product, a compound of Formula 116, is isolated and purified using methods known in the art.

Referring to Scheme IV, Step 3, to a solution of a compound of Formula 116 in an organic solvent such as THF is added di-tert-butyl dicarbonate, an organic base such as triethylamine (16.80 g, 166.06 mmol, 23.11 mL, 1.5 eq) and a nucleophilic catalyst such as 4-dimethylaminopyridine. The mixture is stirred at elevated temperature for 1 h. The product, a compound of Formula 117, is isolated and purified using methods known in the art.

Referring to Scheme IV, Step 4, to a solution of a compound of Formula 117 in a protic solvent such as methanol is added a base such as NH₃/MeOH. The mixture is stirred at ambient temperature for 12 h. The product, a compound of Formula 118, is isolated and purified using methods known in the art.

Referring to Scheme IV, Step 5, to a solution of a compound of Formula 118 in an organic solvent such as THF is added a compound of Formula 119, and an organic acid such as p-toluenesulfonic acid. The mixture is stirred at elevated temperature for 2 h. The product, a compound of Formula 120, is isolated and purified using methods known in the art.

Referring to Scheme IV, Step 6, to a solution of a compound of Formula 120 in an aprotic solvent such as toluene is added a compound of Formula 111, and a catalyst such as Rh(OAc)₂ under an inert atmosphere such as N₂. The mixture is stirred at elevated temperatures for 2 h. The product, a compound of Formula 121, is isolated and purified using methods known in the art.

Referring to Scheme IV, Step 7, to a solution of a compound of Formula 121 in an organic solvent such as THF is added a strong base such as sodium bis(trimethylsilyl)amide at −78° C. The mixture is stirred at −78° C. for 0.5 h. Then to the mixture is added an organic ammonium salt such as tetra-n-butylammonium iodide and an enantioselective reagent such as bromomethyl methyl ether at −78° C. The mixture is stirred at ambient temperature for 2 h. The reaction mixture is quenched with water. The product, a compound of Formula 122, is isolated and purified using methods known in the art.

Referring to Scheme IV, Step 8, to a solution of a compound of Formula 122 in an organic solvent such as THF and H₂O is added a strong base such as LiOH H₂O. The mixture is stirred at an elevated temperature for 12 h. The product, a compound of Formula 123, is isolated and purified using methods known in the art. Individual enantiomers can be separated by using methods known in the art, such as chiral chromatography.

Referring to Scheme IV, Step 9, to a solution of a compound of Formula 123 in an organic base such as DMF is added a coupling agent such as 1-[bis(dimethylamino)methylene]-1H-1,2,3-triazolo[4,5-b]pyridinium 3-oxide hexafluorophosphate in an aprotic solvent such as 4-methylmorpholine. The mixture is stirred at ambient temperature for 1 h. Then a mixture of an alkali metal hydride such as NaH and a compound of Formula 124 in an organic solvent such as DMF is added slowly. The mixture is stirred at ambient temperature for 1 h. The product, a compound of Formula 125, is isolated and purified using methods known in the art.

Referring to Scheme IV, Step 10, to a solution of a compound of Formula 125 in an organic solvent such as dichloromethane is added an acid such as trifluoroacetic acid. The mixture is stirred at ambient temperature for 1 h. The organic layers is concentrated under high vacuum. The mixture is diluted with dichloromethane and then added a Boc deprotecting reagent such as trimethylsilyl iodide and an organic base such as triethylamine. The mixture is stirred at ambient temperature for 1 h. The mixture is concentrated under high vacuum. The mixture is diluted with H₂O and then an acid such as trifluoracetic acid is added. The mixture is stirred at ambient temperature for 2 h. The product, a compound of Formula 126, is isolated and purified using methods known in the art.

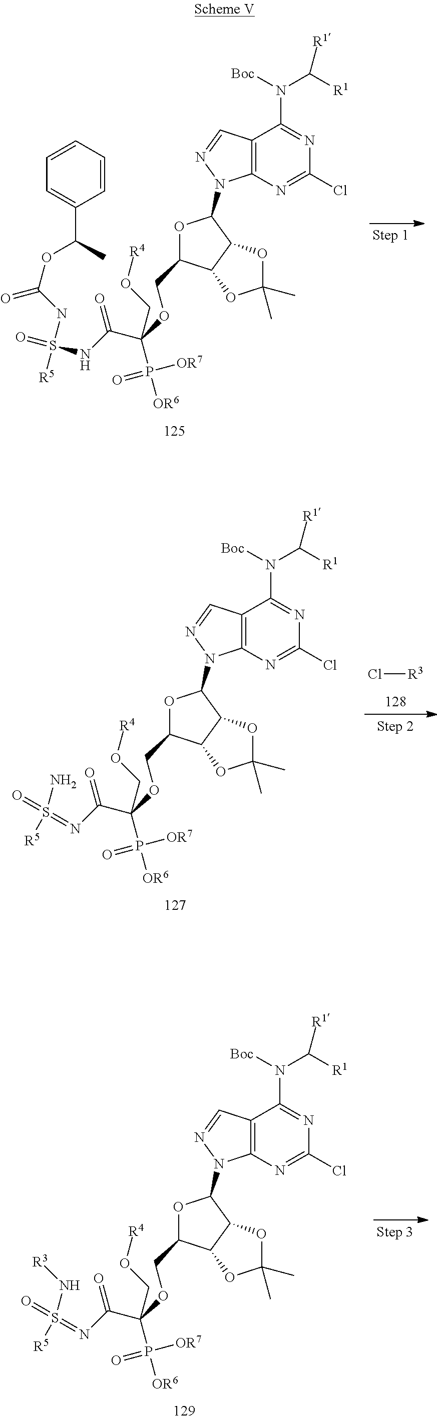

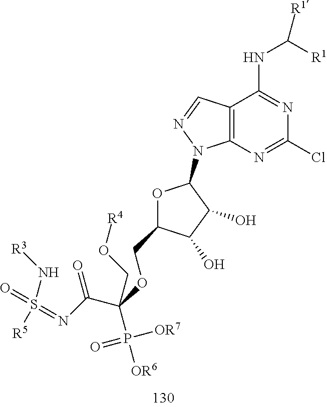

Referring to Scheme V, Step 1, to a solution of a compound of Formula 125 in a protic solvent such as ethanol is added a catalyst such as PtO₂ in an organic solvent such as dichloromethane. The mixture is stirred at ambient temperature under H₂ for 2 h. The product, a compound of Formula 127, is isolated and purified using methods known in the art.

Referring to Scheme V, Step 2, to a solution of a nucleophilic reagent such as a compound of Formula 128 in an organic solvent such as dichloromethane was added an organic base such as triethylamine and a compound of Formula 127. The mixture was stirred at ambient temperature for 0.5 h. The mixture was poured into an acidic solution such as 10% citric acid. The product, a compound of Formula 129, is isolated and purified using methods known in the art.

Referring to Scheme V, Step 3, to a solution of a compound of Formula 127 in an organic solvent such as dichloromethane was added an acid such as trifluoroacetic acid and H₂O. The mixture was stirred at elevated temperature for 1 h, then concentrated. To the resulting mixture was added an organic base like triethylamine and a Boc deprotecting reagent such as trimethylsilyl iodide in an organic solvent such as dichloromethane; the mixture was stirred at ambient temperature for 1 h. The product, a compound of Formula 130, is isolated and purified using methods known in the art.

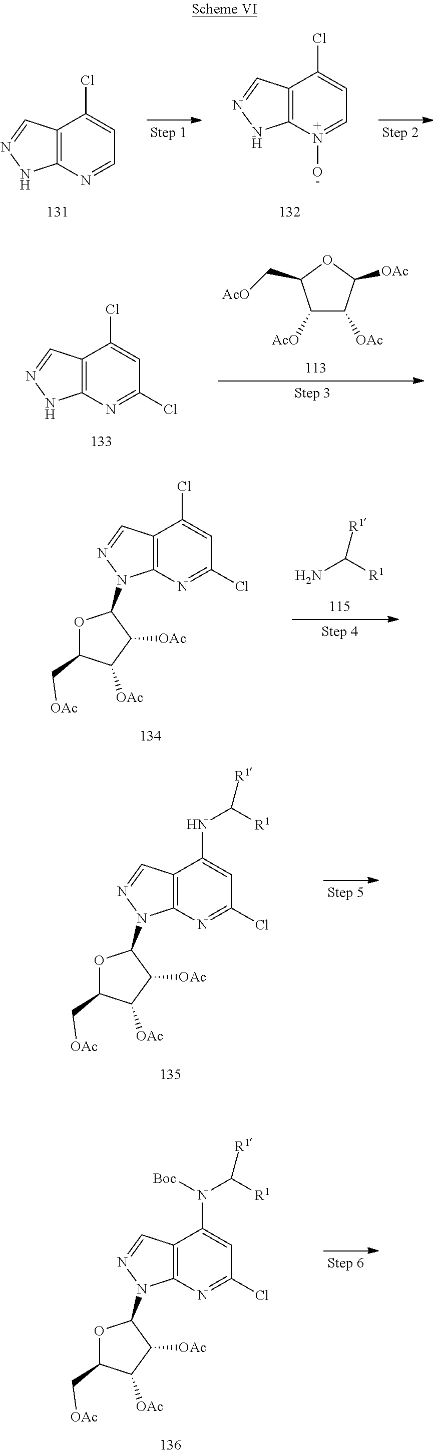

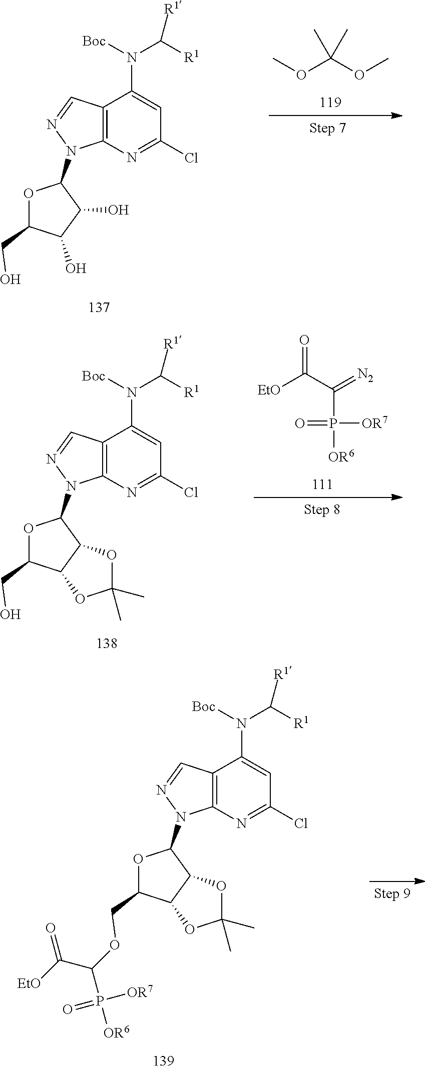

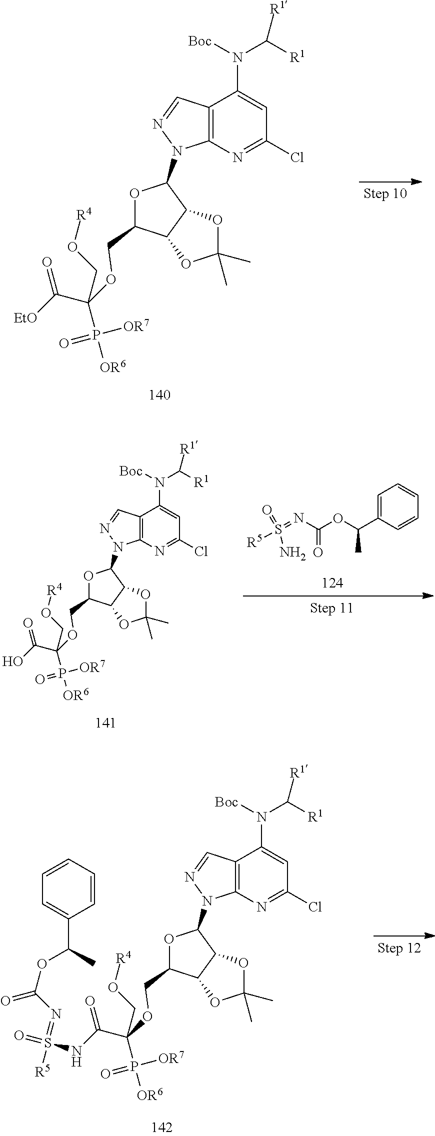

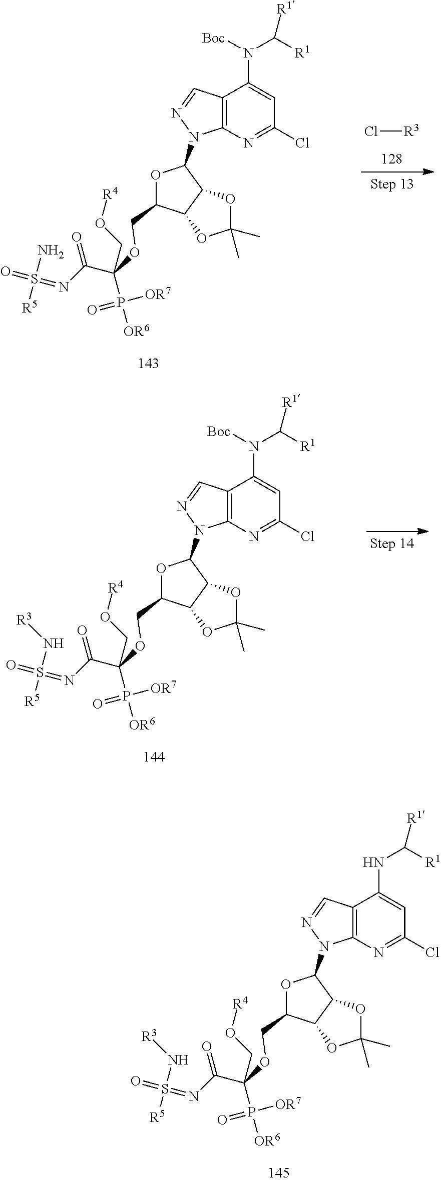

Referring to Scheme VI, Step 1, to a solution of the compound of Formula 131 in an organic solvent such as tetrahydrofuran was added an oxidizing agent such as 3-chlorobenzenecarboperoxoic acid. The mixture was stirred at ambient temperature for 12 h. The product, a compound of Formula 132, is isolated and purified using methods known in the art.

Referring to Scheme VI, Step 2, to a solution of the compound of Formula 132 and a catalyst such as benzyltriethylammonium chloride in an organic solvent such as acetonitrile was added a halogenating agent such as phosphorus oxychloride dropwise. The mixture was stirred at ambient temperature for 12 h. The product, a compound of Formula 133, is isolated and purified using methods known in the art.

Referring to Scheme VI, Step 3, to a solution of the compound of Formula 133 in solvent such as hexamethyldisilazane is added an inorganic salt such as ammonium sulfate. The mixture is stirred at reflux for 3 h. Then the mixture was concentrated in vacuo and then dried under high vacuum. The residue was then diluted with an organic solvent such as acetonitrile. To the crude product is added a solution of the compound of Formula 113 in an organic solvent such as acetonitrile at ambient temperature. Then, trimethylsilyl trifluoromethanesulfonate is added dropwise at 0° C., and the resulting mixture is stirred at ambient temperature for 12 h. The reaction mixture was stirred for an additional hour at 0° C. The product, a compound of Formula 134, is isolated and purified using methods known in the art.

Referring to Scheme VI, Step 4, to a solution of a compound of Formula 134 in an organic solvent such as DMSO is added an organic base, such as triethylamine and a compound of Formula 115. The mixture is stirred at elevated temperature for 12 h. The product, a compound of Formula 135, is isolated and purified using methods known in the art.

Referring to Scheme VI, Step 5, to a solution of a compound of Formula 135 in an organic solvent such as THF is added di-tert-butyl dicarbonate, an organic base such as triethylamine and a nucleophilic catalyst such as 4-dimethylaminopyridine. The mixture is stirred at elevated temperature for 12 h. The product, a compound of Formula 136, is isolated and purified using methods known in the art.

Referring to Scheme VI, Step 6, to a solution of a compound of Formula 136 in a protic solvent such as methanol is added a base such as NH₃/MeOH. The mixture is stirred at ambient temperature for 12 h. The product, a compound of Formula 137, is isolated and purified using methods known in the art.

Referring to Scheme VI, Step 7, to a solution of a compound of Formula 137 in an organic solvent such as DMF is added a compound of Formula 119, and an organic acid such as p-toluenesulfonic acid. The mixture is stirred at elevated temperature for 1 h. The product, a compound of Formula 138, is isolated and purified using methods known in the art.

Referring to Scheme VI, Step 8, to a solution of a compound of Formula 138 in an aprotic solvent such as toluene is added a compound of Formula 111, and a catalyst such as Rh(OAc)₂ under an inert atmosphere such as N₂. The mixture is stirred at elevated temperatures for 1 h. The product, a compound of Formula 139, is isolated and purified using methods known in the art.

Referring to Scheme VI, Step 9, to a solution of a compound of Formula 139 in an organic solvent such as THF is added a strong base such as sodium bis(trimethylsilyl)amide dropwise at −78° C. The mixture is stirred at −78° C. for 0.5 h. Then to the mixture is added an organic ammonium salt such as tetra-n-butylammonium iodide and an enantioselective reagent such as bromomethyl methyl ether at −78° C. The mixture is stirred at ambient temperature for 4 h. The reaction mixture is quenched with by addition of NH₄Cl (35 mL) at 0° C. then diluted with water. The product, a compound of Formula 140, is isolated and purified using methods known in the art.

Referring to Scheme VI, Step 10, to a solution of a compound of Formula 140 in an organic solvent such as THF and H₂O is added a strong base such as LiOH H₂O. The mixture is stirred at an elevated temperature for 36 h. The mixture was acidified by slow addition of an acid such as citric acid. The product, a compound of Formula 141, is isolated and purified using methods known in the art. Individual enantiomers can be separated by using methods known in the art, such as chiral chromatography.

Referring to Scheme VI, Step 11, to a solution of an alkali metal hydride such as NaH in an organic solvent such as DMF was added a compound of Formula 141. The mixture is stirred at ambient temperature for 0.5 h. Then to a mixture of a compound of Formula 124 in an organic solvent such as DMF is added a coupling agent such as 1-[bis(dimethylamino)methylene]-1H-1,2,3-triazolo[4,5-b]pyridinium 3-oxide hexafluorophosphate in an aprotic solvent such as 4-methylmorpholine. The mixture is stirred at ambient temperature for 0.5 h. Then the two mixtures are added together and stirred for 1 h at ambient temperature. The product, a compound of Formula 142, is isolated and purified using methods known in the art.

Referring to Scheme VI, Step 12, to a solution of a compound of Formula 142 in a organic solvent such as THF is added a catalyst such as PtO₂ in an organic solvent such as chloroform. The mixture is stirred at ambient temperature under H₂ for 2 h. The product, a compound of Formula 143, is isolated and purified using methods known in the art.

Referring to Scheme VI, Step 13, to a solution of a compound of Formula 143 in an organic solvent such as dichloromethane was added an organic base such as triethylamine and a nucleophilic reagent such as a compound of Formula 128. The mixture was stirred at ambient temperature for 2 h. The reaction mixture was quenched by addition water at 0° C. The product, a compound of Formula 144, is isolated and purified using methods known in the art.

Referring to Scheme VI, Step 14, to a solution of a compound of Formula 144 in an organic solvent such as dichloromethane was added an acid such as trifluoroacetic acid and H₂O. The mixture was stirred at ambient temperature for 10 minutes, then concentrated. To the resulting mixture was added an organic base like triethylamine in an organic solvent such as ethyl acetate. The product, a compound of Formula 145, is isolated and purified using methods known in the art.

The invention is further illustrated by the following examples. All IUPAC names were generated using CambridgeSoft's ChemDraw 19.0.

The following chemical abbreviations are used in the synthetic methods and are defined as such.

| Abbreviation | Meaning |
|---|---|
| DMSO | dimethyl sulfoxide |
| TsN3 | Tosyl azide |
| HMDS | hexamethyldisilazane |
| EtOH | ethanol |
| TEA | triethylamine |
| THF | tetrahydrofuran |
| DMAP | dimethylaminopyridine |
| PTSA | p-Toluenesulfonic acid |
| OAc | acetate |
| HPLC | High-performance liquid chromatography |
| TFA | trifluoroacetic acid |
| ACN | acetonitrile |
| TBAI | Tetra-n-butylammonium iodide |
| NaHMDS | Sodium bis(trimethylsilyl)amide |
| MeOH | methanol |
| SFC | Supercritical fluid chromatography |
| DMA | dimethylamine |
| HATU | 1-[Bis(dimethylamino)methylene]-1H-1,2,3- |
| | triazolo[4,5-b]pyridinium 3-oxide |
| | hexafluorophosphate |
| DCM | dichloromethane |
| TMSI | Trimethylsilyl iodide |
| t-BuOH | t-butanol |

### Example 1

### ((2S)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

### Step 1

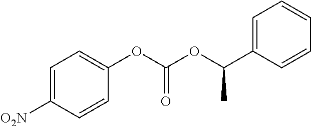 [SMILES: C[C@@H](OC(=O)Oc1ccc([N+](=O)[O-])cc1)c1ccccc1]

### (R)-4-Nitrophenyl (1-phenylethyl) carbonate

To a solution of (R)-1-phenylethan-1-ol (20 g, 163.71 mmol, 19.80 mL, 1 eq) in dichloromethane (200 mL) was added pyridine (13.60 g, 171.90 mmol, 13.87 mL, 1.05 eq) and (4-nitrophenyl) carbonochloridate (33.00 g, 163.71 mmol, 1 eq) at 0° C. The mixture was stirred at ambient temperature for 16 h. The mixture was poured into water (500 mL), the aqueous layer was extracted with dichloromethane (3×200 mL), the combined organic layer was washed with saturated NaCl aqueous solution (3×50 mL), the combined organic layer was dried with anhydrous Na₂SO₄, filtered and concentrated to give a residue. The residue was purified by column chromatography (SiO₂, Petroleum ether/Ethyl acetate=0/1 to 10/1) to give (R)-4-nitrophenyl (1-phenylethyl) carbonate (30 g, yield 63.79%) as colorless oil.

¹H NMR: DMSO-d₆; δ=1.63 (d, J=6.63 Hz, 3H), 5.84 (q, J=6.59 Hz, 1H), 7.33-7.49 (m, 5H), 7.51-7.57 (m, 2H), 8.27-8.32 (m, 2H).

### Step 2

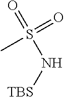

### N-(tert-Butyldimethylsilyl)methanesulfonamide

To a solution of methanesulfonamide (10 g, 105.13 mmol, 1 eq) in CHCl₃ (100 mL) was added tert-butyl(chloro)di(methyl)silane (47.54 g, 315.39 mmol, 38.65 mL, 3 eq) and triethylamine (31.91 g, 315.39 mmol, 43.90 mL, 3 eq) at 0° C. The mixture was stirred at ambient temperature for 16 h. The mixture was poured into water (50 mL), the aqueous layer was extracted with dichloromethane (3×20 mL), the combined organic layer was washed with saturated NaCl aqueous solution (2×5 mL), the combined organic layer was dried with anhydrous Na₂SO₄, filtered and concentrated to give a residue. The residue was purified by column chromatography (SiO₂, Petroleum ether/Ethyl acetate=10/1 to 1/1) to give N-(tert-butyldimethylsilyl)methanesulfonamide (19 g, yield 100.00%) as a white solid.

¹H NMR: 400 MHz, DMSO-d₆; δ=0.16 (s, 6H), 0.89 (s, 9H), 2.90 (s, 3H), 7.06 (s, 1H).

### Step 3

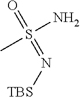

### N′-(tert-Butyldimethylsilyl)methanesulfonimidamide

To a mixture of 1,1,1,2,2,2-hexachloroethane (8.79 g, 37.11 mmol, 4.20 mL, 1.11 eq) in CHCl₃ (30 mL) was added PPh₃ (9.73 g, 37.11 mmol, 1.11 eq) and the mixture was stirred at 70° C. for 18 h, and then triethylamine (5.07 g, 50.15 mmol, 6.98 mL, 1.5 eq) was added at ambient temperature, the resulting mixture was stirred at ambient temperature for 0.5 h, and then N-(tert-butyldimethylsilyl)methanesulfonamide (7 g, 33.43 mmol, 1 eq) was added at 0° C., the resulting mixture was stirred at 0° C. for 0.5 h, NH₃ was bubbled into the solution at 0° C. for 6 minutes, the resulting mixture was stirred at ambient temperature for 1 h. The mixture was poured into water (20 mL) the aqueous layer was extracted with dichloromethane (3×20 mL), the combined organic layer was washed with brine (3×10 mL), the combined organic layer was dried with anhydrous Na₂SO₄, filtered and concentrated in vacuum. The residue was purified by flash silica gel chromatography (petroleum ether/ethyl acetate=20:1 to 1:1) to give N′-(tert-butyldimethylsilyl)methanesulfonimidamide (2.8 g, yield 40.19%) as a white solid.

¹H NMR: 400 MHz, DMSO-d₆; δ=0.01 (d, J=2.75 Hz, 6H), 0.85 (s, 9H), 2.92 (s, 3H), 6.18 (s, 2H).

### Step 4

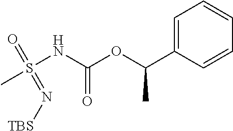

### (R)-1-Phenylethyl (N-(tert-butyldimethylsilyl)-S-methylsulfonimidoyl)carbamate

To a solution of N′-(tert-butyldimethylsilyl)methanesulfonimidamide (5.4 g, 25.91 mmol, 1 eq) in THF (60 mL) was added n-BuLi (2.5 M, 22.80 mL, 2.2 eq), the mixture was stirred at −78° C. for 0.5 h. To the mixture was added (R)-4-nitrophenyl (1-phenylethyl) carbonate (9.68 g, 33.69 mmol, 1.3 eq) at −78° C., the mixture was stirred at 0° C. for 1 h. The mixture was poured into saturated NH₄Cl (10 mL), the aqueous layer was extracted with ethyl acetate (3×5 mL), the combined organic layer was washed with brine (2×5 mL), the combined organic layer was dried with anhydrous Na₂SO₄, filtered and concentrated in vacuum to give (R)-1-phenylethyl (N-(tert-butyldimethylsilyl)-S-methylsulfonimidoyl)carbamate (9 g, yield 97.41%) as a colorless oil.

### Step 5

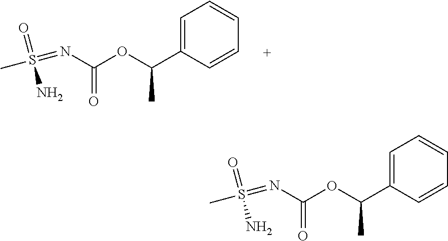 [SMILES: C[C@@H](OC(=O)N=[S@@](C)(N)=O)c1ccccc1 ; C[C@@H](OC(=O)N=[S@](C)(N)=O)c1ccccc1]

### (R)-1-Phenylethyl ((R)-amino(methyl)(oxo)-λ⁶-sulfaneylidene)carbamate and (R)-1-Phenylethyl ((S)-amino(methyl)(oxo)-λ⁶-sulfaneylidene)carbamate

To a solution of (R)-1-phenylethyl (N-(tert-butyldimethylsilyl)-S-methylsulfonimidoyl)carbamate (9 g, 25.24 mmol, 1 eq) in THF (90 mL) was added tetra-n-butylammonium fluoride (1 M, 50.48 mL, 2 eq), the mixture was stirred at ambient temperature for 1 h. The mixture was poured into saturated NH₄Cl (100 mL), the aqueous layer was extracted with ethyl acetate (3×50 mL), the combined organic layer was washed with brine (2×50 mL), the combined organic layer was dried with anhydrous Na₂SO₄, filtered and concentrated in vacuum. The residue was purified by prep-HPLC (column: Welch Xtimate C18 250*70 mm #10 um; mobile layer: [water(NH4HCO3)-ACN]; B %: 10%-40%, 20 min), and then separated by SFC (column: DAICEL CHIRALPAK IC(250 mm*30 mm, 10 um); mobile layer: [0.1% NH3H2O IPA]; B %: 50%-50%, 8 min) to give (R)-1-phenylethyl ((R)-amino(methyl)(oxo)-λ⁶-sulfaneylidene)carbamate (0.9 g, 3.71 mmol, 45.00% yield) as a white solid and (R)-1-phenylethyl ((S)-amino(methyl)(oxo)-λ⁶-sulfaneylidene)carbamate (0.9 g, 3.71 mmol, 45.00% yield) as a white solid.

¹H NMR: (R)-1-phenylethyl ((R)-amino(methyl)(oxo)-λ⁶-sulfaneylidene)carbamate; 400 MHz, DMSO-d₆; δ=1.42 (d, J=6.63 Hz, 3H), 3.22 (s, 3H), 5.62 (q, J=6.55 Hz, 1H), 7.24-7.31 (m, 3H), 7.31-7.38 (m, 4H).

¹H NMR: (R)-1-phenylethyl ((S)-amino(methyl)(oxo)-λ⁶-sulfaneylidene)carbamate; DMSO-d₆; δ=1.43 (d, J=6.63 Hz, 3H), 3.20 (s, 3H), 5.63 (q, J=6.59 Hz, 1H), 7.21-7.40 (m, 7H).

SFC: Rt (P1): 2.301 min; Rt (P2): 2.721 min. Column: ChiralPak IC-3,100×4.6 mm I.D., 3 um, Mobile layer: A: CO₂ B:IPA(0.1% IPAm, v/v), Flow rate: 3.4 mL/min, Column temp.: 35° C., ABPR:2000 psi.

### Step 6

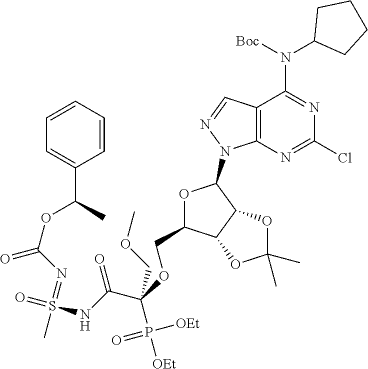 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N[S@@](C)(=O)=NC(=O)O[C@H](C)c1ccccc1]

### tert-Butyl (6-chloro-1-((3aR,4R,6R,6aR)-6-((((S)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate

To a solution of (S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-2-(diethoxyphosphoryl)-3-methoxypropanoic acid (50 mg, 66.83 μmol, 1 eq) in DMF (0.5 mL) was added 1-[bis(dimethylamino)methylene]-1H-1,2,3-triazolo[4,5-b]pyridinium 3-oxide hexafluorophosphate (30.49 mg, 80.20 μmol, 1.2 eq) and 4-methylmorpholine (20.28 mg, 200.49 μmol, 22.04 μL, 3 eq), the mixture was stirred for 1 h, and then (R)-1-phenylethyl ((R)-amino(methyl)(oxo)-λ⁶-sulfaneylidene)carbamate (19.43 mg, 80.20 μmol, 1.2 eq) and NaH (8.02 mg, 200.49 μmol, 60% purity, 3 eq) in DMF (0.5 mL) was added, the resulting mixture was stirred at ambient temperature for 1 h. The mixture was poured into saturated NH₄Cl (10 mL), the aqueous layer was extracted with ethyl acetate (3×5 mL), the combined organic layer was washed with brine (2×5 mL), the combined organic layer was dried with anhydrous Na₂SO₄, filtered and concentrated in vacuum to give tert-butyl (6-chloro-1-((3aR,4R,6R,6aR)-6-((((S)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (60 mg, yield 92.32%) as a colorless oil.

LCMS (ESI+): m/z 972.5 [M+H]⁺, Rt: 1.091 min.

### Step 7

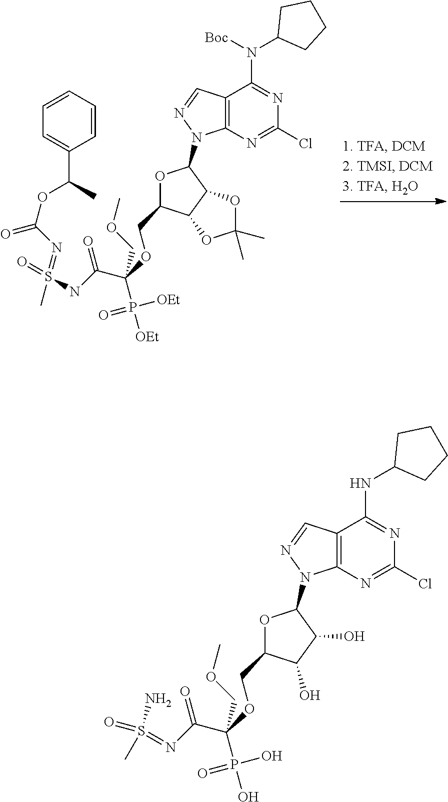 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N[S@@](C)(=O)=NC(=O)O[C@H](C)c1ccccc1 ; COC[C@@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O]

### ((2S)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

To a solution of tert-butyl (6-chloro-1-((3aR,4R,6R,6aR)-6-((((S)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (60 mg, 61.70 μmol, 1 eq) in dichloromethane (1 mL) was added trifluoroacetic acid (308.00 mg, 2.70 mmol, 0.2 mL, 43.78 eq), the resulting mixture was stirred at ambient temperature for 1 h. The mixture was filtered and concentrated under reduced pressure to give a residue. To the mixture was added triethylamine (111.79 mg, 1.10 mmol, 153.76 μL, 20 eq) and trimethylsilyl iodide (165.79 mg, 828.54 μmol, 112.78 μL, 15 eq) in dichloromethane (1 mL), the resulting mixture was stirred at ambient temperature for 1 h. The mixture was filtered and concentrated under reduced pressure to give a residue. To the mixture was added H₂O (0.5 mL) and trifluoroacetic acid (0.5 mL), the resulting mixture was stirred at ambient temperature for 1 h. The mixture was concentrated and the residue was purified by prep-HPLC (column: Waters XBridge BEH C18 100*30 mm*10 um; mobile layer: [water(NH3H2O+NH4HCO3)-ACN]; B %: 5%-35%, 8 min) to give ((2S)-1-((amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid (6 mg, yield 18.24%, purity 100%) as a white solid.

¹H NMR: 400 MHz, CD₃OD; δ=1.58-1.74 (m, 4H), 1.77-1.89 (m, 2H), 2.08-2.16 (m, 2H), 3.32-3.32 (m, 3H), 3.36 (s, 3H), 3.85-3.92 (m, 1H), 4.05-4.18 (m, 2H), 4.20-4.29 (m, 2H), 4.50-4.61 (m, 1H), 4.70 (t, J=4.89 Hz, 2H), 6.22 (d, J=4.27 Hz, 1H), 8.14 (s, 1H).

LCMS (ESI+): m/z 628.2 [M+H]⁺, Rt:1.472 min.

### Example 2

### ((2R)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

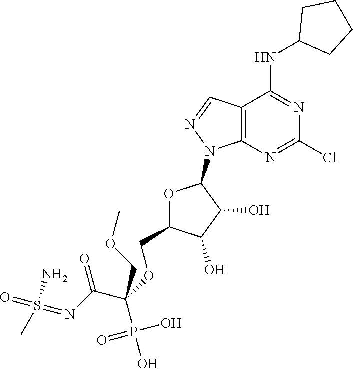 [SMILES: COC[C@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O]

((2R)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid was prepared analogously to Example 1.

¹H NMR: 400 MHz, CD₃OD; δ=1.55-1.74 (m, 4H), 1.76-1.85 (m, 2H), 2.06-2.14 (m, 2H), 3.34 (s, 3H), 3.38 (s, 3H), 3.86-3.94 (m, 1H), 3.95-4.02 (m, 1H), 4.04-4.11 (m, 1H), 4.20-4.31 (m, 2H), 4.49-4.58 (m, 1H), 4.72-4.77 (m, 1H), 5.00-5.03 (m, 1H), 6.20-6.26 (m, 1H), 8.16 (s, 1H).

LCMS (ESI+): m/z 628.2 [M+H]⁺, Rt:1.473 min.

### Example 3

### ((2S)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

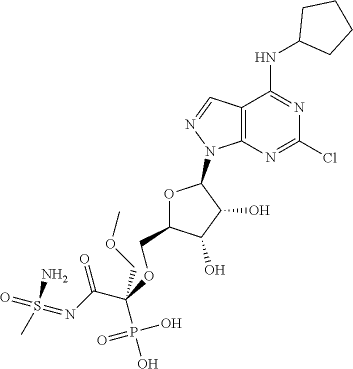 [SMILES: COC[C@@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O]

((2S)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid was prepared analogously to Example 1.

¹H NMR: 400 MHz, MeOD; δ=1.57-1.71 (m, 4H), 1.77-1.85 (m, 2H), 2.05-2.13 (m, 2H), 3.35 (s, 3H), 3.37 (s, 3H), 3.89 (dd, J=10.51, 2.88 Hz, 1H), 4.05-4.10 (m, 1H), 4.12-4.17 (m, 1H), 4.20-4.26 (m, 2H), 4.51-4.59 (m, 2H), 4.66 (br s, 1H), 6.21 (br d, J=4.38 Hz, 1H), 8.12 (s, 1H).

LCMS (ESI+): m/z 628.1 [M+H]⁺, Rt:1.471 min.

### Example 4

### ((2R)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

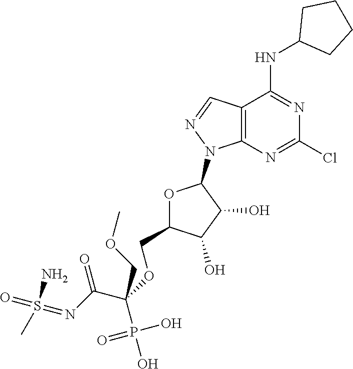 [SMILES: COC[C@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O]

((2R)-1-((Amino(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid was prepared analogously to Example 1.

¹H NMR: 400 MHz, MeOD; δ=1.57-1.71 (m, 4H), 1.78-1.85 (m, 2H), 2.06-2.15 (m, 2H), 3.30 (br s, 3H), 3.33 (s, 3H), 3.87 (dd, J=10.82, 2.69 Hz, 1H), 3.96-4.03 (m, 1H), 4.06-4.12 (m, 1H), 4.22-4.30 (m, 2H), 4.52-4.57 (m, 1H), 4.76 (br d, J=1.88 Hz, 1H), 4.99 (t, J=5.38 Hz, 1H), 6.23 (d, J=5.63 Hz, 1H), 8.15 (s, 1H).

LCMS (ESI+): m/z 628.1 [M+H]⁺, Rt:1.481 min.

### Example 5

### ((S)-1-(((R)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

### Step 1

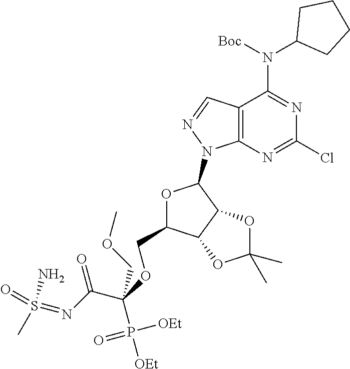 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@](C)(N)=O]

### tert-Butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a solution of tert-butyl (6-chloro-1-((3aR,4R,6R,6aR)-6-((((S)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (50 mg, 51.42 μmol, 1 eq) in EtOH (1 mL) was added PtO₂ (11.68 mg, 51.42 μmol, 1 eq) and CHCl₃ (12.28 mg, 102.83 μmol, 8.29 μL, 2 eq). The mixture was stirred at ambient temperature under H₂ (15 psi) for 2 h. The mixture was filtered, and the filtrate was concentrated to give the crude product, which was purified by prep-TLC (Ethyl acetate/Methanol=10/1) to give tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (20 mg, yield 47.19%) as a white solid.

¹H NMR: 400 MHz, MeOD-d₄; δ=1.28 (dd, J=7.19, 3.94 Hz, 6H), 1.40 (s, 3H), 1.57 (s, 9H), 1.59 (s, 3H), 1.61-1.70 (m, 2H), 1.88-2.00 (m, 5H), 2.01 (s, 3H), 2.04-2.09 (m, 1H), 3.34 (s, 3H), 3.69 (dd, J=9.19, 6.19 Hz, 1H), 3.83-3.86 (m, 1H), 3.88-3.99 (m, 2H), 4.06-4.11 (m, 1H), 4.13-4.20 (m, 4H), 4.46 (ddd, J=7.91, 6.10, 1.75 Hz, 1H), 5.32 (dd, J=6.00, 1.88 Hz, 1H), 5.48 (dd, J=6.00, 1.25 Hz, 1H), 6.49 (s, 1H), 8.07 (s, 1H).

### Step 2

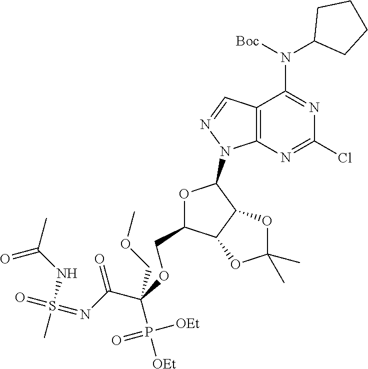 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(=O)NC(C)=O]

### tert-Butyl (1-((3aR,4R,6R,6aR)-6-((((S)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a solution of tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (20 mg, 24.26 μmol, 1 eq) in dichloromethane (1 mL) was added triethylamine (4.91 mg, 48.53 μmol, 6.75 μL, 2 eq) and 4-dimethylaminopyridine (296.43 ug, 2.43 μmol, 0.1 eq), acetic anhydride (2.72 mg, 26.69 μmol, 2.50 μL, 1.1 eq). The mixture was stirred at 30° C. for 1 h. The mixture was poured into water (10 mL), extracted with dichloromethane (3×5 mL), separated, the organic layer was washed with brine (3 mL), dried over Na₂SO₄ and concentrated to give tert-butyl (1-((3aR,4R,6R,6aR)-6-((((S)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (21 mg, yield 99.91%) as colorless oil.

LCMS (ESI+): m/z 866.4 [M+H]⁺, Rt:1.011 min.

### Step 3

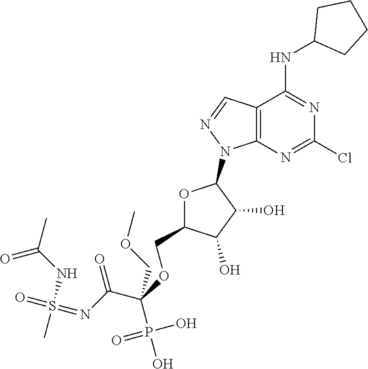 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

### ((S)-1-(((R)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

To a solution of tert-butyl (1-((3aR,4R,6R,6aR)-6-((((S)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (21 mg, 24.24 μmol, 1 eq) in dichloromethane (0.4 mL) was added trifluoroacetic acid (0.04 mL) and H₂O (0.83 mg, 46.18 μmol, 0.83 μL, 2 eq). The mixture was stirred at 30° C. for 1 h. The mixture was concentrated. To the mixture was added triethylamine (95.09 mg, 939.71 μmol, 130.80 μL, 40 eq) and trimethylsilyl iodide (141.02 mg, 704.78 μmol, 95.93 μL, 30 eq) in dichloromethane (0.4 mL), the mixture was stirred at ambient temperature for 1 h. The mixture was filtered, the filtrate was concentrated, and the residue was purified by prep-HPLC (column: Waters XBridge BEH C18 100*30 mm*10 um; mobile layers: [water (NH₃H₂O+NH₄HCO₃)-ACN]; B %: 5%-35%, 8 min) to give ((S)-1-(((R)-acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid (2 mg, yield 12.64%, purity 95.4%) as a white solid.

¹H NMR: 400 MHz, MeOD-d₄; δ=1.55-1.72 (m, 4H), 1.76-1.85 (m, 2H), 1.95-1.96 (s, 3H), 2.05-2.21 (m, 2H), 3.34 (s, 3H), 3.43-3.48 (m, 3H), 3.90 (br dd, J=9.94, 2.81 Hz, 1H), 4.10 (br d, J=4.25 Hz, 2H), 4.20-4.31 (m, 2H), 4.52-4.56 (m, 1H), 4.64 (br d, J=4.25 Hz, 2H), 6.22 (d, J=4.50 Hz, 1H), 8.14 (s, 1H).

LCMS (ESI+): m/z 670.2 [M+H]⁺, Rt:1.379 min.

### Example 6

### ((R)-1-(((R)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

### Step 1

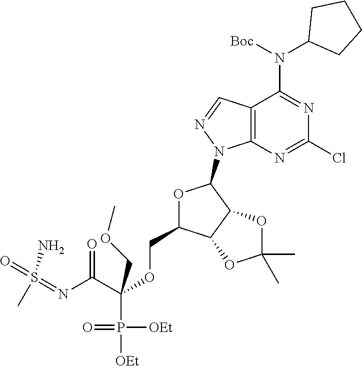 [SMILES: CCOP(=O)(OCC)[C@@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@](C)(N)=O]

### tert-Butyl (1-((3aR,4R,6R,6aR)-6-((((R)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a mixture of tert-butyl (6-chloro-1-((3aR,4R,6R,6aR)-6-((((R)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (120 mg, 123.40 μmol, 1 eq) in EtOH (2 mL) was added PtO₂ (28.02 mg, 123.40 μmol, 1 eq) and CHCl₃ (44.19 mg, 370.20 μmol, 29.86 μL, 3 eq), the resulting mixture was stirred at ambient temperature under H₂ (15 Psi) for 12 h. The mixture was filtered and concentrated in vacuum. The residue was purified by prep-TLC (EtOAc/MeOH=20/1) to give tert-butyl (1-((3aR,4R,6R,6aR)-6-((((R)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (80 mg, yield 78.65%) as a colorless oil.

¹H NMR: 400 MHz, MeOD-d₄; δ=1.24 (s, 3H), 1.28-1.31 (m, 3H), 1.40 (s, 3H), 1.56 (s, 9H), 1.58-1.65 (m, 5H), 1.85-2.00 (m, 4H), 2.02-2.09 (m, 2H), 3.33 (s, 3H), 3.40 (s, 3H), 3.69 (dd, J=9.01, 5.50 Hz, 1H), 3.83-3.92 (m, 1H), 3.92-3.98 (m, 2H), 4.12-4.24 (m, 4H), 4.41-4.48 (m, 1H), 4.59 (s, 2H), 5.03 (t, J=8.69 Hz, 1H), 5.34 (dd, J=6.07, 1.94 Hz, 1H), 5.46 (dd, J=6.00, 1.25 Hz, 1H), 6.48 (s, 1H), 8.07 (s, 1H).

### Step 2

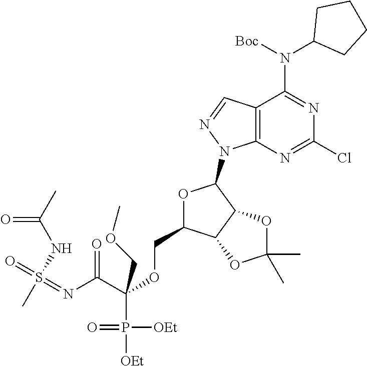 [SMILES: CCOP(=O)(OCC)[C@@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(=O)NC(C)=O]

### tert-Butyl (1-((3aR,4R,6R,6aR)-6-((((R)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a mixture of tert-butyl (1-((3aR,4R,6R,6aR)-6-((((R)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (40 mg, 48.53 μmol, 1 eq) in dichloromethane (1 mL) was added triethylamine (9.82 mg, 97.05 μmol, 13.51 μL, 2 eq), DMAP (592.85 ug, 4.85 μmol, 0.1 eq) and Ac₂O (5.45 mg, 53.38 μmol, 5.00 μL, 1.1 eq), the resulting mixture was stirred at ambient temperature for 1 h. The mixture was poured into water (5 mL), the aqueous layers was extracted with dichloromethane (3×3 mL), the combined organic layers was washed with brine (2×3 mL), the combined organic layers was dried with anhydrous Na₂SO₄, filtered and concentrated in vacuum to give tert-butyl (1-((3aR,4R,6R,6aR)-6-((((R)-1-(((R)-acetamido(methyl)(oxo)-_i-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (40 mg, yield 95.15%) as a colorless oil.

LCMS (ESI+): m/z 865.9 [M+H]⁺, Rt:0.893 min.

### Step 3

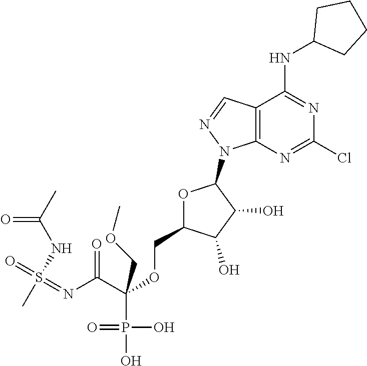 [SMILES: COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

### ((R)-1-(((R)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

To a mixture of tert-butyl (1-((3aR,4R,6R,6aR)-6-((((R)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (40 mg, 46.17 μmol, 1 eq) in dichloromethane (1 mL) was added H₂O (1.66 mg, 92.35 μmol, 1.66 μL, 2 eq) and trifluoroacetic acid (154.00 mg, 1.35 mmol, 100.00 μL, 29.25 eq), the resulting mixture was stirred at ambient temperature for 1 h. The mixture was filtered and concentrated under reduced pressure to give a residue. To the residue was added triethylamine (83.73 mg, 827.42 μmol, 115.17 μL, 20 eq) and trimethylsilyl iodide (124.17 mg, 620.56 μmol, 84.47 μL, 15 eq) in dichloromethane (1 mL), the resulting mixture was stirred at ambient temperature for 1 h. The mixture was filtered and concentrated under reduced pressure to give a residue. The residue was purified by prep-HPLC (column: Waters XBridge BEH C18 100*30 mm*10 um; mobile layers: [water(NH3H2O+NH4HCO3)-ACN]; B %: 1%-30%, 8 min) to give ((R)-1-(((R)-acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid (11 mg, 16.29 μmol, 39.37% yield, 99.2% purity) as a white solid.

¹H NMR: 400 MHz, MeOD-d₄; δ=1.51-1.74 (m, 4H), 1.76-1.87 (m, 2H), 1.99 (s, 3H), 2.04-2.16 (m, 2H), 3.32 (br s, 3H), 3.43 (s, 3H), 3.91 (dd, J=10.69, 2.81 Hz, 1H), 4.02-4.13 (m, 2H), 4.17-4.28 (m, 2H), 4.52-4.57 (m, 1H), 4.68 (t, J=4.88 Hz, 1H), 4.88-4.88 (m, 1H), 6.21 (d, J=4.50 Hz, 1H), 8.14 (s, 1H).

LCMS (ESI+): m/z 670.3 [M+H]⁺, Rt:1.385 min.

### Example 7

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-((methoxycarbonyl)amino)(methyl)(oxo)-λ⁶-sulfaneylidene)amino)-1-oxopropan-2-yl)phosphonic Acid

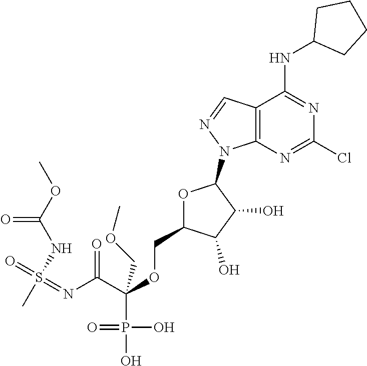 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O]

### Step 1

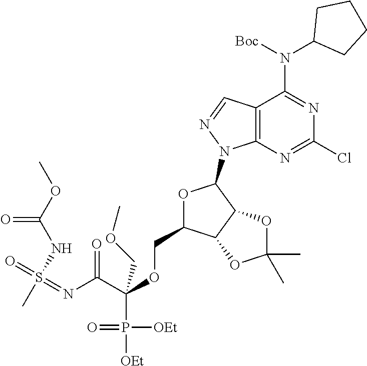 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(=O)NC(=O)OC]

### tert-Butyl (6-chloro-1-((3aR,4R,6R,6aR)-6-((((S)-2-(diethoxyphosphoryl)-3-methoxy-1-(((R)-((methoxycarbonyl)amino)(methyl)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate

To a solution of methyl carbonochloridate (0.08 g, 846.59 μmol, 65.57 μL, 46.52 eq) in dichloromethane (0.5 mL) was added triethylamine (9.21 mg, 90.99 μmol, 12.66 μL, 5 eq) and tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (15 mg, 18.20 μmol, 1 eq). The mixture was stirred at ambient temperature for 0.5 h. The mixtures was poured into 10% citric acid (10 mL), extracted with dichloromethane (3×5 mL), separated, the organic layer was washed with brine (3 mL), dried over Na₂SO₄ and concentrated to give tert-butyl (6-chloro-1-((3aR,4R,6R,6aR)-6-((((S)-2-(diethoxyphosphoryl)-3-methoxy-1-(((R)-((methoxycarbonyl)amino)(methyl)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (16 mg, yield 99.65%) as a yellow oil.

LCMS (ESI+): m/z 882.1 [M+H]⁺, Rt:1.033 min.

### Step 2

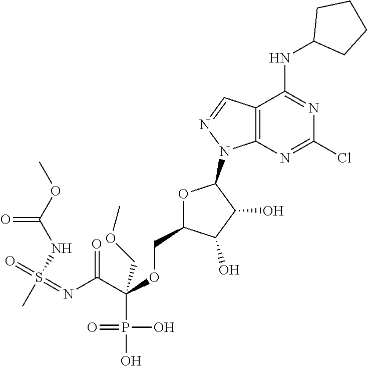 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O]

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-((methoxycarbonyl)amino)(methyl)(oxo)-λ⁶-sulfaneylidene)amino)-1-oxopropan-2-yl)phosphonic Acid

((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-((methoxycarbonyl)amino)(methyl)(oxo)-λ⁶-sulfaneylidene)amino)-1-oxopropan-2-yl)phosphonic acid was prepared analogously to Example 5.

¹H NMR: 400 MHz, MeOD-d₄; δ=1.54-1.75 (m, 4H), 1.76-1.89 (m, 2H), 2.05-2.17 (m, 2H), 3.35 (s, 3H), 3.44-3.49 (m, 3H), 3.60 (s, 3H), 3.94 (br dd, J=11.07, 2.19 Hz, 1H), 3.99-4.07 (m, 1H), 4.12 (br dd, J=9.82, 3.44 Hz, 1H), 4.20-4.29 (m, 2H), 4.51-4.59 (m, 1H), 4.60-4.68 (m, 1H), 4.80 (br s, 1H), 6.24 (d, J=4.88 Hz, 1H), 8.17 (s, 1H).

LCMS (ESI+): m/z 686.3 [M+H]⁺, Rt:1.410 min.

### Example 8

### ((R)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-((methoxycarbonyl)amino)(methyl)(oxo)-λ⁶-sulfaneylidene)amino)-1-oxopropan-2-yl)phosphonic Acid

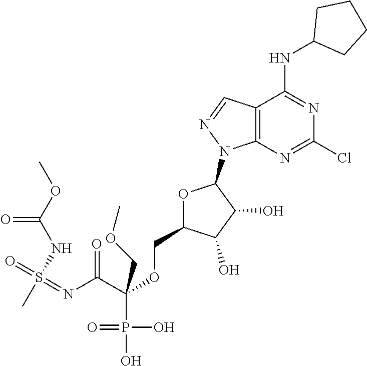 [SMILES: COC[C@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O]

((R)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-((methoxycarbonyl)amino)(methyl)(oxo)-λ⁶-sulfaneylidene)amino)-1-oxopropan-2-yl)phosphonic acid was prepared analogously to Example 7.

¹H NMR: 400 MHz, MeOD-d₄; δ=1.53-1.75 (m, 4H), 1.75-1.88 (m, 2H), 2.02-2.17 (m, 2H), 3.32-3.32 (m, 3H), 3.36-3.45 (m, 3H), 3.61 (s, 3H), 3.92 (br dd, J=10.82, 2.31 Hz, 1H), 3.96-4.06 (m, 1H), 4.08-4.16 (m, 1H), 4.16-4.29 (m, 2H), 4.55 (dt, J=13.51, 7.00 Hz, 1H), 4.66 (br t, J=4.82 Hz, 1H), 4.87-4.88 (m, 1H), 6.18-6.29 (m, 1H), 8.14 (s, 1H).

LCMS (ESI+): m/z 686.3 [M+H]⁺, Rt:1.405 min.

### Example 9

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-methyl(3-methylureido)(oxo)-λ⁶-sulfaneylidene)amino)-1-oxopropan-2-yl)phosphonic Acid

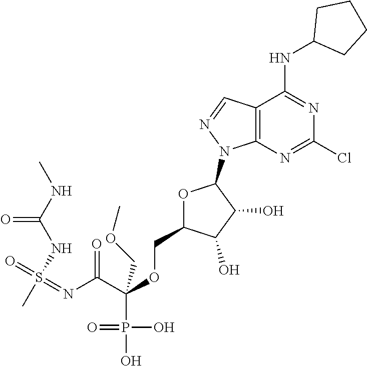 [SMILES: CNC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

### Step 1

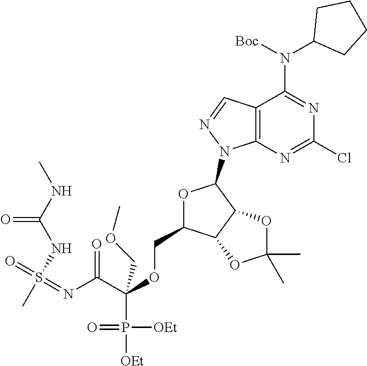 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(=O)NC(=O)NC]

### tert-Butyl (6-chloro-1-((3aR,4R,6R,6aR)-6-((((S)-2-(diethoxyphosphoryl)-3-methoxy-1-(((R)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate

To a solution of tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (30 mg, 36.40 μmol, 1 eq) and N-methylcarbamoyl chloride (10.21 mg, 109.19 μmol, 3 eq) in dichloromethane (0.5 mL) was added triethylamine (14.73 mg, 145.58 μmol, 20.26 μL, 4 eq). The mixture was stirred at ambient temperature for 18 h. To the mixture was added water (10 mL), the aqueous layer was extracted with ethyl acetate (3×5 mL), the combined organic layer was washed with saturated NaCl aqueous solution (2×2 mL), the combined organic layer was dried with anhydrous Na₂SO₄, filtered, and concentrated to give tert-butyl (6-chloro-1-((3aR,4R,6R,6aR)-6-((((S)-2-(diethoxyphosphoryl)-3-methoxy-1-(((R)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (30 mg, yield 93.53%) as a yellow oil.

LCMS (ESI+): m/z 881.5 [M+H]⁺, Rt: 0.983 min.

### Step 2

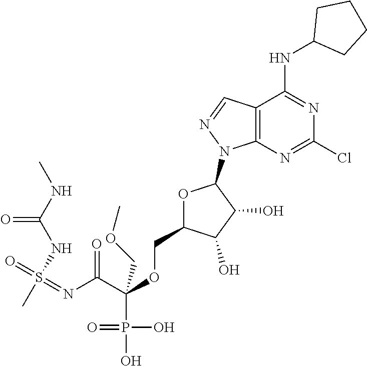 [SMILES: CNC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-methyl(3-methylureido)(oxo)-λ⁶-sulfaneylidene)amino)-1-oxopropan-2-yl)phosphonic Acid

To a solution of tert-butyl (6-chloro-1-((3aR,4R,6R,6aR)-6-((((S)-2-(diethoxyphosphoryl)-3-methoxy-1-(((R)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (30 mg, 34.04 μmol, 1 eq) in dichloromethane (1 mL) was added trifluoroacetic acid (3.88 mg, 34.04 μmol, 2.52 μL, 1 eq). The mixture was stirred at ambient temperature for 2 h. The mixture was filtered and concentrated under reduced pressure. The residue was dissolved in dichloromethane (1 mL) and to the solution was added triethylamine (68.26 mg, 674.63 μmol, 93.90 μL, 20 eq) and trimethylsilyl iodide (101.24 mg, 505.97 μmol, 68.87 μL, 15 eq). The mixture was stirred at ambient temperature for 1 h. The mixture was filtered and concentrated under reduced pressure. The residue was purified by prep-HPLC (column: Waters XBridge BEH C18 100*30 mm*10 um; mobile phase: [water(NH₃H₂O+NH₄HCO₃)-ACN]; B %: 1%-30%, 8 min) to give ((S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-(((R)-methyl(3-methylureido)(oxo)-λ⁶-sulfaneylidene)amino)-1-oxopropan-2-yl)phosphonic acid (6 mg, yield 24.98%, purity 96.2%) as a white solid.

¹H NMR: 400 MHz, MeOD-d₄; δ=1.55-1.74 (m, 4H), 1.80 (br d, J=6.00 Hz, 2H), 2.03-2.17 (m, 2H), 2.65 (s, 3H), 3.34 (s, 3H), 3.36 (br s, 3H), 3.94 (dd, J=10.88, 3.00 Hz, 1H), 4.02-4.13 (m, 2H), 4.19 (dd, J=10.69, 4.82 Hz, 1H), 4.26 (q, J=4.92 Hz, 1H), 4.50-4.58 (m, 1H), 4.64-4.67 (m, 1H), 4.76 (br s, 1H), 6.21 (d, J=4.13 Hz, 1H), 8.14 (s, 1H).

LCMS (ESI+): m/z 685.1 [M+H]⁺, Rt:1.943 min.

### Example 10

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((S)-methyl(3-methylureido)(oxo)-λ⁶-sulfaneylidene)amino)-1-oxopropan-2-yl)phosphonic Acid

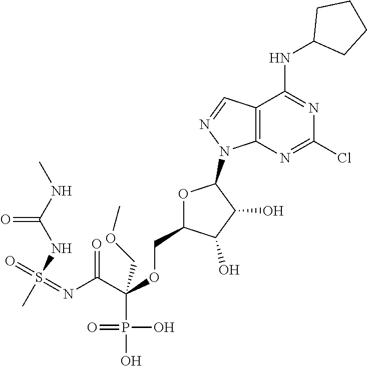 [SMILES: CNC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

### Step 1

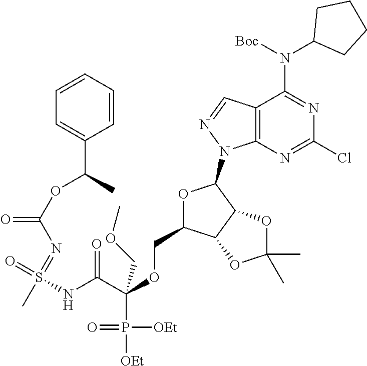 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N[S@](C)(=O)=NC(=O)O[C@H](C)c1ccccc1]

### tert-Butyl (6-chloro-1-((3aR,4R,6R,6aR)-6-((((S)-2-(diethoxyphosphoryl)-3-methoxy-1-oxo-1-((R)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate

To a solution of (S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-2-(diethoxyphosphoryl)-3-methoxypropanoic acid (400 mg, 534.65 μmol, 1 eq) in DMF (4 mL) was added 1-[bis(dimethylamino)methylene]-1H-1,2,3-triazolo[4,5-b]pyridinium 3-oxide hexafluorophosphate (264.28 mg, 695.04 μmol, 1.3 eq) and 4-methylmorpholine (162.23 mg, 1.60 mmol, 176.34 μL, 3 eq). The mixture was stirred at ambient temperature for 1 h. Then a solution of NaH (64.15 mg, 1.60 mmol, 60% purity, 3 eq) and (R)-1-phenylethyl (amino(methyl)(oxo)-λ⁶-sulfaneylidene)carbamate (194.31 mg, 801.97 μmol, 1.5 eq) in DMF (4 mL) was added slowly. The mixture was stirred at ambient temperature for 1 h. The reaction was poured into water (80 mL). The mixture was extracted with ethyl acetate (3×10 mL). The combined organic layers were washed with brine and dried over Na₂SO₄. The organic layer was concentrated to give crude tert-butyl (6-chloro-1-((3aR,4R,6R,6aR)-6-((((S)-2-(diethoxyphosphoryl)-3-methoxy-1-oxo-1-((R)—N—(((R)-1-phenylethoxy)carbonyl)methylsulfonoamidimidamido)propan-2-yl)oxy)methyl)-2,2-dimethyltetrahydrofuro[3,4-d]1[1,3]dioxol-4-yl)-1H-pyrazolo[3,4-d]pyrimidin-4-yl)(cyclopentyl)carbamate (400 mg, yield 76.94%) as a brown solid.

LCMS (ESI+): m/z 972.4 [M+H]⁺, Rt:0.942 min.

### Step 2

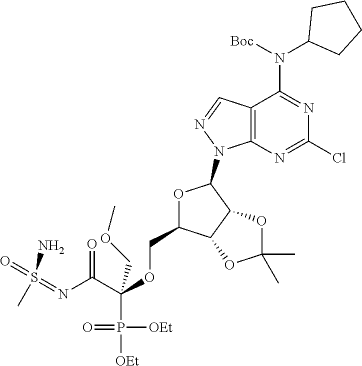 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(N)=O]

### tert-Butyl (1-((3aR,4R,6R,6aR)-6-((((S)-1-(((R)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a mixture of PtO₂ (93.41 mg, 411.34 μmol, 1 eq) in EtOH (4 mL) was added tert-butyl (6-chloro-1-((3aR,4R,6R,6aR)-6-((((S)-2-(diethoxyphosphoryl)-3-methoxy-1-oxo-1-((R)—N—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (400 mg, 411.34 μmol, 1 eq) and CHCl₃ (147.31 mg, 1.23 mmol, 99.54 μL, 3 eq) under H₂ (15 Psi). The mixture was stirred at ambient temperature for 12 h. The mixture was filtered. The organic layer was concentrated. The residue was purified by prep-TLC (SiO2, Ethyl acetate:Methanol=20:1) to give tert-butyl (1-((3aR,4R,6R,6aR)-6-((((S)-1-(((R)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (200 mg, yield 59.06%) as a white solid.

¹H NMR: 400 MHz, MeOD-d₄; δ=1.25-1.29 (m, 6H), 1.40 (s, 3H), 1.56 (s, 9H), 1.58-1.63 (m, 5H), 1.89-2.00 (m, 5H), 2.02-2.05 (m, 1H), 3.30 (br s, 3H), 3.33 (s, 3H), 3.66 (dd, J=9.01, 6.25 Hz, 1H), 3.84-3.92 (m, 3H), 4.16 (tdd, J=7.16, 5.00, 2.06 Hz, 4H), 4.42-4.48 (m, 1H), 5.03 (quin, J=8.66 Hz, 1H), 5.31 (dd, J=6.00, 1.75 Hz, 1H), 5.47 (dd, J=6.07, 1.06 Hz, 1H), 6.48 (s, 1H), 8.07 (s, 1H).

### Step 3

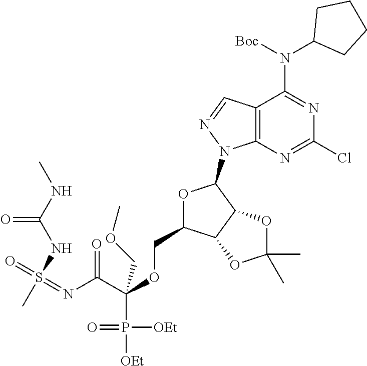 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@](C)(=O)NC(=O)NC]

### tert-Butyl (6-chloro-1-((3aR,4R,6R,6aR)-6-((((S)-2-(diethoxyphosphoryl)-3-methoxy-1-(((S)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate

To a solution of tert-butyl (1-((3aR,4R,6R,6aR)-6-((((S)-1-(((R)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (30 mg, 36.40 μmol, 1 eq) in dichloromethane (1 mL) was added N-methylcarbamoyl chloride (5.11 mg, 54.59 μmol, 1.5 eq) and triethylamine (7.37 mg, 72.79 μmol, 10.13 μL, 2 eq). The mixture was stirred at ambient temperature for 18 h. The mixture was poured into water (10 mL), the aqueous layer was extracted with ethyl acetate (3×5 mL), the combined organic layer was washed with saturated NaCl aqueous solution (2×2 mL), the combined organic layer was dried with anhydrous Na₂SO₄, filtered and concentrated to give tert-butyl (6-chloro-1-((3aR,4R,6R,6aR)-6-((((S)-2-(diethoxyphosphoryl)-3-methoxy-1-(((S)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (25 mg, yield 77.94%) as a yellow oil.

LCMS (ESI+): m/z 881.2 [M+H]⁺, Rt:0.662 min.

### Step 4

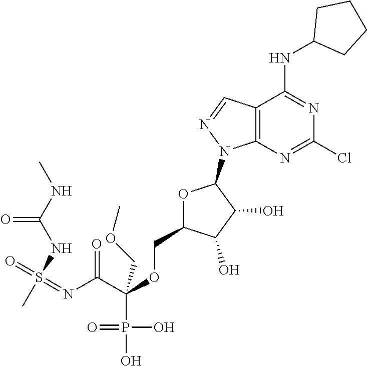 [SMILES: CNC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((S)-methyl(3-methylureido)(oxo)-λ⁶-sulfaneylidene)amino)-1-oxopropan-2-yl)phosphonic Acid

To a solution of tert-butyl (6-chloro-1-((3aR,4R,6R,6aR)-6-((((S)-2-(diethoxyphosphoryl)-3-methoxy-1-(((S)-methyl(3-methylureido)(oxo)-λ⁶-sulfaneylidene)amino)-1-oxopropan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (25 mg, 28.37 μmol, 1 eq) in dichloromethane (1 mL) was added trifluoroacetic acid (154.00 mg, 1.35 mmol, 100.00 μL, 47.61 eq). The mixture was stirred at ambient temperature for 3 h. The mixture was filtered and concentrated under reduced pressure to give a residue. The mixture in dichloromethane (1 mL) was added triethylamine (54.61 mg, 539.70 μmol, 75.12 μL, 20 eq) and trimethylsilyl iodide (80.99 mg, 404.78 μmol, 55.10 μL, 15 eq). The mixture was stirred at ambient temperature for 1 h. The mixture was filtered and concentrated under reduced pressure to give a residue. The residue was purified by prep-HPLC (column: Waters XBridge BEH C18 100*30 mm*10 um; mobile phase: [water(NH₃H₂O+NH₄HCO₃)-ACN]; B %: 1%-25%, 8 min) to give ((S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-(((S)-methyl(3-methylureido)(oxo)-_i-sulfaneylidene)amino)-1-oxopropan-2-yl)phosphonic acid (6.7 mg, yield 34.79%, purity 96.0%) as a white solid.

¹H NMR: 400 MHz, MeOD-d₄; δ=1.56-1.71 (m, 4H), 1.75-1.84 (m, 2H), 2.04-2.14 (m, 2H), 2.65 (s, 3H), 3.33 (s, 3H), 3.43 (s, 3H), 3.91-3.96 (m, 1H), 4.01-4.07 (m, 1H), 4.10-4.15 (m, 1H), 4.18 (br d, J=4.00 Hz, 1H), 4.25 (q, J=4.54 Hz, 1H), 4.51-4.55 (m, 2H), 4.66 (br s, 1H), 6.22 (d, J=4.75 Hz, 1H), 8.18 (s, 1H).

LCMS (ESI+): m/z 683.1 [M−H]⁻, Rt:1.950 min.

### Example 11

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonic Acid

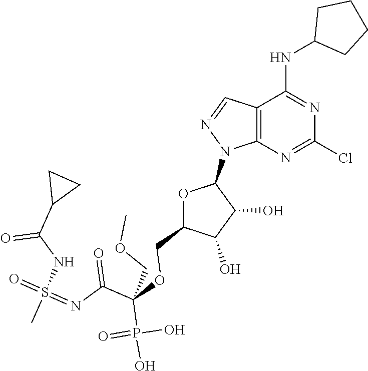

((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-(((R)-cyclopropanecarboxamido(methyl)(oxo)-_i-sulfaneylidene)amino)-3-methoxy-1-oxopropan-2-yl)phosphonic acid was prepared analogously to Example 9.

¹H NMR: 400 MHz, MeOD-d₄; δ=0.67-0.81 (m, 2H), 0.82-0.91 (m, 2H), 1.45-1.56 (m, 1H), 1.56-1.76 (m, 4H), 1.76-1.87 (m, 2H), 2.05-2.15 (m, 2H), 3.34 (s, 3H), 3.42 (br s, 3H), 3.92 (dd, J=10.82, 3.06 Hz, 1H), 4.09 (br d, J=3.63 Hz, 2H), 4.16-4.23 (m, 1H), 4.25 (q, J=4.71 Hz, 1H), 4.52-4.57 (m, 1H), 4.64 (br d, J=5.00 Hz, 1H), 4.77 (br s, 1H), 6.22 (d, J=4.38 Hz, 1H), 8.14 (s, 1H).

LCMS (ESI+): m/z 696.2 [M+H]⁺, Rt:2.011 min.

### Example 12

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-(((S)-cyclopropanecarboxamido(methyl)(oxo)-λ⁶-sulfaneylidene)amino)-3-methoxy-1-oxopropan-2-yl)phosphonic Acid

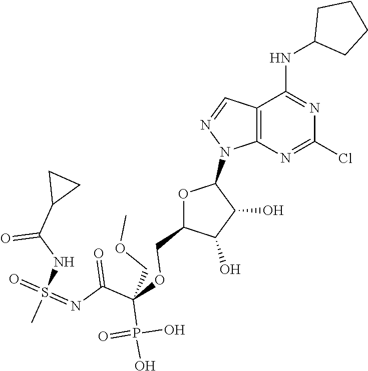

((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-(((S)-cyclopropanecarboxamido(methyl)(oxo)-λ⁶-sulfaneylidene)amino)-3-methoxy-1-oxopropan-2-yl)phosphonic acid was prepared analogously to Example 10.

¹H NMR: 400 MHz, MeOD-d₄; δ=0.64-0.72 (m, 2H), 0.79-0.86 (m, 2H), 1.55-1.73 (m, 5H), 1.75-1.86 (m, 2H), 2.04-2.16 (m, 2H), 3.32 (br s, 3H), 3.34 (s, 3H), 3.92 (dd, J=10.88, 3.25 Hz, 1H), 4.05-4.19 (m, 3H), 4.25 (q, J=5.54 Hz, 1H), 4.54 (br s, 1H), 4.63 (br d, J=5.50 Hz, 1H), 4.69 (br dd, J=5.00, 3.38 Hz, 1H), 6.19 (d, J=3.38 Hz, 1H), 8.10-8.13 (m, 1H).

LCMS (ESI+): m/z 696.2 [M+H]⁺, Rt:2.019 min.

### Example 13

### ((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxo)(propionamido)-λ⁶-sulfaneylidene)amino)-1-oxopropan-2-yl)phosphonic Acid

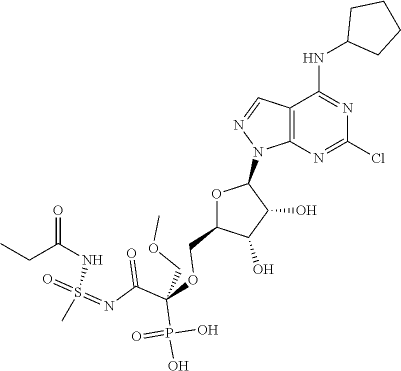

((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxo)(propionamido)-λ⁶-sulfaneylidene)amino)-1-oxopropan-2-yl)phosphonic acid was prepared analogously to Example 9.

¹H NMR: 400 MHz, MeOD-d₄; δ=1.05 (t, J=7.50 Hz, 3H), 1.57-1.72 (m, 4H), 1.77-1.85 (m, 2H), 2.06-2.14 (m, 2H), 2.23 (q, J=7.46 Hz, 2H), 3.34 (s, 3H), 3.47 (s, 3H), 3.88-3.93 (m, 1H), 4.10 (d, J=4.88 Hz, 2H), 4.19-4.24 (m, 1H), 4.24-4.28 (m, 1H), 4.51-4.57 (m, 1H), 4.64 (br t, J=4.82 Hz, 1H), 4.79 (br s, 1H), 6.22 (d, J=4.38 Hz, 1H), 8.15 (s, 1H).

LCMS (ESI+): m/z 684.2 [M+H]⁺, Rt:2.012 min.

### Example 14

### ((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxo)(propionamido)-λ⁶-sulfaneylidene)amino)-1-oxopropan-2-yl)phosphonic Acid

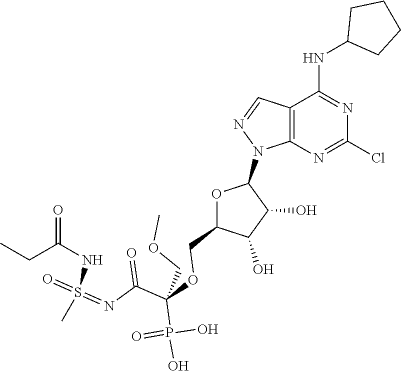

((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxo)(propionamido)-λ⁶-sulfaneylidene)amino)-1-oxopropan-2-yl)phosphonic acid was prepared analogously to Example 10.

¹H NMR: 400 MHz, MeOD-d₄; δ=1.09 (t, J=7.57 Hz, 3H), 1.59-1.70 (m, 4H), 1.78-1.84 (m, 2H), 2.07-2.14 (m, 2H), 2.28-2.34 (m, 2H), 3.34 (s, 3H), 3.58 (s, 3H), 3.87-3.92 (m, 1H), 4.12 (br d, J=3.75 Hz, 2H), 4.22-4.27 (m, 2H), 4.54 (br s, 1H), 4.67 (br s, 1H), 4.73 (br s, 1H), 6.22 (d, J=4.75 Hz, 1H), 8.15 (s, 1H).

LCMS (ESI+): m/z 684.2 [M+H]⁺, Rt:1.948 min.

### Example 15

### ((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-((isobutyramido(methyl)(oxo)-λ⁶-sulfaneylidene)amino)-3-methoxy-1-oxopropan-2-yl)phosphonic Acid

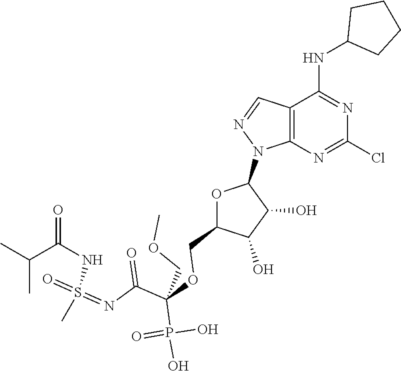

((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-((isobutyramido(methyl)(oxo)-λ⁶-sulfaneylidene)amino)-3-methoxy-1-oxopropan-2-yl)phosphonic acid was prepared analogously to Example 9.

¹H NMR: 400 MHz, MeOH-d₄; δ=1.07 (t, J=6.84 Hz, 6H), 1.57-1.65 (m, 2H), 1.66-1.74 (m, 2H), 1.78-1.85 (m, 2H), 2.06-2.14 (m, 2H), 2.33-2.42 (m, 1H), 3.35 (s, 3H), 3.46 (s, 3H), 3.92 (dd, J=11.04, 2.89 Hz, 1H), 4.10 (d, J=5.14 Hz, 2H), 4.21 (br dd, J=10.98, 5.08 Hz, 1H), 4.25-4.28 (m, 1H), 4.52-4.57 (m, 1H), 4.63-4.65 (m, 1H), 4.77 (br s, 1H), 6.23 (d, J=4.52 Hz, 1H), 8.15 (s, 1H)

LCMS (ESI+): m/z 698.2 [M+H]⁺, Rt:1.998 min.

### Example 16

### ((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-((isobutyramido(methyl)(oxo)-λ⁶-sulfaneylidene)amino)-3-methoxy-1-oxopropan-2-yl)phosphonic Acid

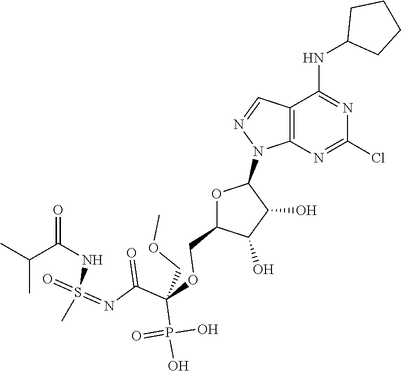

((2S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-1-((isobutyramido(methyl)(oxo)-λ⁶-sulfaneylidene)amino)-3-methoxy-1-oxopropan-2-yl)phosphonic acid was prepared analogously to Example 10.

¹H NMR: 400 MHz, MeOD-d₄; δ=1.11 (dd, J=6.88, 2.38 Hz, 6H), 1.28-1.32 (m, 1H), 1.58-1.68 (m, 4H), 1.79-1.83 (m, 2H), 2.06-2.14 (m, 3H), 2.41-2.48 (m, 1H), 3.34 (s, 3H), 3.55 (s, 3H), 3.90-3.94 (m, 1H), 4.12 (br d, J=5.88 Hz, 2H), 4.22-4.27 (m, 2H), 4.54 (br s, 1H), 4.67 (br s, 1H), 4.76 (br s, 1H), 6.22 (br d, J=4.38 Hz, 1H), 8.16 (s, 1H).

LCMS (ESI+): m/z 698.2 [M+H]⁺, Rt:1.990 min.

### Example 17

### ((S)-1-(((S)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

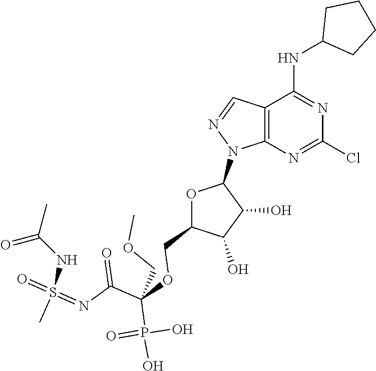 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(C)=O)P(=O)(O)O]

((S)-1-(((S)-Acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic acid was prepared analogously to Example 5.

¹H NMR: 400 MHz, MeOD-d₄; δ=1.58-1.71 (m, 4H), 1.78-1.84 (m, 2H), 2.02 (s, 3H), 2.07-2.13 (m, 2H), 3.34 (s, 3H), 3.53 (s, 3H), 3.90 (dd, J=10.88, 2.75 Hz, 1H), 4.11-4.14 (m, 2H), 4.19-4.25 (m, 2H), 4.51-4.57 (m, 1H), 4.66 (t, J=5.00 Hz, 1H), 4.78 (br s, 1H), 6.21 (d, J=4.13 Hz, 1H), 8.13 (s, 1H).

LCMS (ESI+): m/z 670.1 [M+H]⁺, Rt:1.942 min.

### Example 18

### ((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((S)-((methoxycarbonyl)amino)(methyl)(oxo)-λ⁶-sulfaneylidene)amino)-1-oxopropan-2-yl)phosphonic Acid

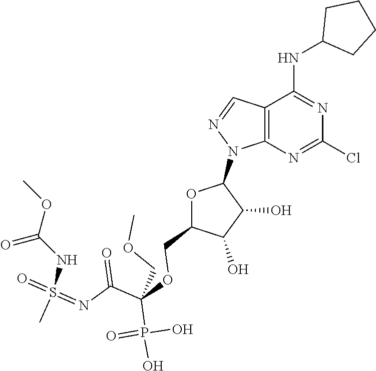 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)OC)P(=O)(O)O]

((S)-2-(((2R,3S,4R,5R)-5-(6-Chloro-4-(cyclopentylamino)-1H-pyrazolo[3,4-d]pyrimidin-1-yl)-3,4-dihydroxytetrahydrofuran-2-yl)methoxy)-3-methoxy-1-(((S)-((methoxycarbonyl)amino)(methyl)(oxo)-λ⁶-sulfaneylidene)amino)-1-oxopropan-2-yl)phosphonic acid was prepared analogously to Example 7.

¹H NMR: 400 MHz, MeOD-d₄; δ=1.57-1.66 (m, 2H), 1.67-1.75 (m, 2H), 1.79-1.87 (m, 2H), 2.08-2.16 (m, 2H), 3.35 (br s, 3H), 3.39 (s, 3H), 3.61 (s, 3H), 3.94 (dd, J=10.38, 3.13 Hz, 1H), 4.13 (br d, J=5.38 Hz, 2H), 4.19 (dd, J=10.94, 5.19 Hz, 1H), 4.27 (q, J=5.04 Hz, 1H), 4.56 (br s, 1H), 4.68 (br s, 1H), 4.75 (br s, 1H), 6.22 (d, J=3.63 Hz, 1H), 8.14 (s, 1H).

### Example 19

### ((S)-2-(((2R,3S,4R,5R)-5-(6-Chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonic Acid

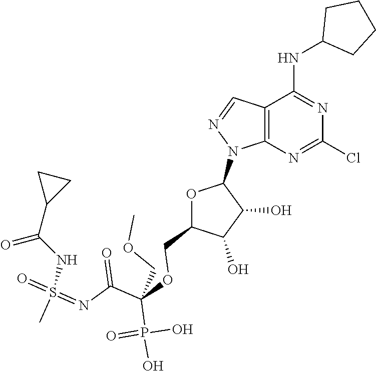 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(O)O]

### Step 1

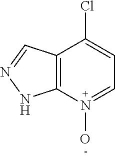

### 4-Chloro-1H-pyrazolo[3,4-b]pyridine 7-oxide

To a solution of 4-chloro-1H-pyrazolo[3,4-b]pyridine (24 g, 156.28 mmol, 1 eq) in tetrahydrofuran (300 mL) was added 3-chlorobenzenecarboperoxoic acid (67.42 g, 390.70 mmol, 2.5 eq). The mixture was stirred at 20° C. for 12 h. The reaction mixture was diluted with methyl tertiary butyl ether (500 mL). The mixture was triturated in methyl tertiary butyl ether. The resulting solid was collected by filtration to give 4-chloro-1H-pyrazolo[3,4-b]pyridine 7-oxide (24 g, 141.54 mmol, yield 90.56%) as a light-yellow solid.

¹H NMR: 400 MHz, DMSO-d6; δ=7.19-7.38 (m, 1H), 7.84-7.96 (m, 1H), 8.36 (br d, J=6.48 Hz, 1H), 14.66-15.07 (m, 1H).

### Step 2

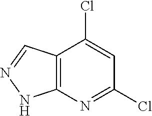 [SMILES: Clc1cc(Cl)c2cn[nH]c2n1]

### 4,6-Dichloro-1H-pyrazolo[3,4-b]pyridine

To a solution of 4-chloro-1H-pyrazolo[3,4-b]pyridine 7-oxide (24 g, 141.54 mmol, 1 eq) and benzyltriethylammonium chloride (16.12 g, 70.77 mmol, 0.5 eq) in acetonitrile (300 mL) was added phosphorus oxychloride (43.40 g, 283.07 mmol, 26.31 mL, 2 eq) dropwise. The mixture was stirred at 20° C. for 12 h. The reaction was quenched by addition of 100 mL of warm water and then the pH was adjusted to 8 by progressively adding solid NaHCO₃. The mixture was collected by filtration. The filtered cake was washed with water (50 mL) to give 4,6-dichloro-1H-pyrazolo[3,4-b]pyridine (24 g, 127.65 mmol, yield 90.19%) as a yellow solid.

¹H NMR: 400 MHz, DMSO-d6; δ=7.54 (s, 1H), 8.30 (s, 1H), 13.97-14.50 (m, 1H).

### Step 3

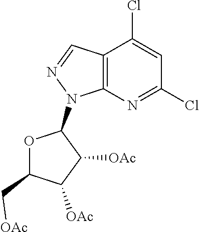 [SMILES: CC(=O)OC[C@H]1O[C@@H](n2ncc3c(Cl)cc(Cl)nc32)[C@H](OC(C)=O)[C@@H]1OC(C)=O]

### (2R,3R,4R,5R)-2-(Acetoxymethyl)-5-(4,6-dichloro-1H-pyrazolo[3,4-b]pyridin-1-yl)tetrahydrofuran-3,4-diyl Diacetate

To a mixture of 4,6-dichloro-1H-pyrazolo[3,4-b]pyridine (24 g, 127.65 mmol, 1 eq) in 1,1,1,3,3,3,-hexamethyldisilazane (30 mL) was added ammonium sulfate (1.69 g, 12.77 mmol, 952.98 μL, 0.1 eq). The mixture was stirred for 3 h at 130° C. Then the mixture was concentrated in vacuo and then dried under high vacuum. The residue was then diluted with acetonitrile (60 mL) and [(2R,3R,4R,5S)-3,4,5-triacetoxytetrahydrofuran-2-yl]methyl acetate (48.75 g, 153.18 mmol, 1.2 eq) was added. This mixture was cooled to 0° C. and trimethylsilyl trifluoromethanesulfonate (31.21 g, 140.42 mmol, 25.37 mL, 1.1 eq) was added dropwise. The mixture was stirred at 20° C. for 12 h. The reaction mixture was stirred for an additional hour at 0° C. and then quenched by slow addition of saturated aqueous NaHCO₃ (200 mL) at 0° C. The mixture was diluted with ethyl acetate (200 mL). The aqueous phase was extracted with ethyl acetate (3×200 mL), and then the combined organic extracts were washed with brine, dried over Na₂SO₄, and concentrated in vacuo to give (2R,3R,4R,5R)-2-(acetoxymethyl)-5-(4,6-dichloro-1H-pyrazolo[3,4-b]pyridin-1-yl)tetrahydrofuran-3,4-diyl diacetate (24 g, 53.78 mmol, 42.13% yield) as a white solid.

¹H NMR: 400 MHz, DMSO-d6; δ=1.98 (s, 3H), 2.08 (s, 3H), 2.11 (s, 3H), 4.08 (d, J=5.00 Hz, 1H), 4.35 (dd, J=12.13, 3.25 Hz, 1H), 4.39-4.49 (m, 1H), 5.66 (t, J=5.75 Hz, 1H), 5.88 (dd, J=5.19, 3.44 Hz, 1H), 6.50 (d, J=3.25 Hz, 1H), 7.74 (s, 1H), 8.57 (s, 1H).

### Step 4

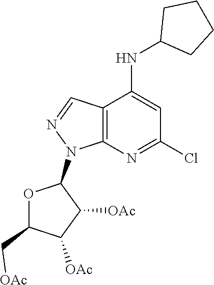 [SMILES: CC(=O)OC[C@H]1O[C@@H](n2ncc3c(NC4CCCC4)cc(Cl)nc32)[C@H](OC(C)=O)[C@@H]1OC(C)=O]

### (2R,3R,4R,5R)-2-(Acetoxymethyl)-5-(6-chloro-4-(cyclopentylamino)-1H-pyrazolo[3,4-b]pyridin-1-yl)tetrahydrofuran-3,4-diyl Diacetate

To a solution of (2R,3R,4R,5R)-2-(acetoxymethyl)-5-(4,6-dichloro-1H-pyrazolo[3,4-b]pyridin-1-yl)tetrahydrofuran-3,4-diyl diacetate (24 g, 53.78 mmol, 1 eq) in DMSO (250 mL) was added cyclopentanamine (5.04 g, 59.16 mmol, 5.84 mL, 1.1 eq) and triethylamine (19.05 g, 188.24 mmol, 26.20 mL, 3.5 eq). The mixture was stirred at 80° C. for 12 h. The residue was diluted with water (2 L) and extracted with ethyl acetate (3×500 mL). The combined organic layers were washed with brine (2 L), dried over Na₂SO₄, filtered, and concentrated under reduced pressure to give (2R,3R,4R,5R)-2-(Acetoxymethyl)-5-(6-chloro-4-(cyclopentylamino)-1H-pyrazolo[3,4-b]pyridin-1-yl)tetrahydrofuran-3,4-diyl diacetate (24 g, 48.49 mmol, yield 90.16%) as brown oil.

LCMS (ESI+): m/z 495.2 [M+H]⁺, Rt: 0.798 min.

### Step 5

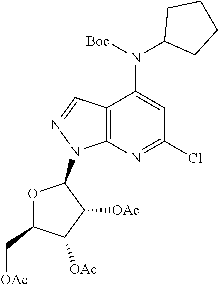 [SMILES: CC(=O)OC[C@H]1O[C@@H](n2ncc3c(N(C(=O)OC(C)(C)C)C4CCCC4)cc(Cl)nc32)[C@H](OC(C)=O)[C@@H]1OC(C)=O]

### (2R,3R,4R,5R)-2-(Acetoxymethyl)-5-(4-((tert-butoxycarbonyl)(cyclopentyl)amino)-6-chloro-1H-pyrazolo[3,4-b]pyridin-1-yl)tetrahydrofuran-3,4-diyl Diacetate

To a solution of (2R,3R,4R,5R)-2-(Acetoxymethyl)-5-(6-chloro-4-(cyclopentylamino)-1H-pyrazolo[3,4-b]pyridin-1-yl)tetrahydrofuran-3,4-diyl diacetate (9 g, 18.18 mmol, 1 eq) in THF (50 mL) was added Boc₂O (19.84 g, 90.92 mmol, 20.89 mL, 5 eq), triethylamine (4.60 g, 45.46 mmol, 6.33 mL, 2.5 eq) and 4-dimethylaminopyridine (2.22 g, 18.18 mmol, 1 eq). The mixture was stirred at 80° C. for 2 h. Two additional vials were set up as described above. All three reaction mixtures were combined for purification. The mixture was diluted with water (100 mL) and extracted with ethyl acetate (3×100 mL). The combined organic layer was washed with brine (300 mL), dried over Na₂SO₄, filtered and concentrated under reduced pressure. The residue was purified by column chromatography (SiO₂, Petroleum ether/Ethyl acetate=0:1 to 1:1) to give (2R,3R,4R,5R)-2-(acetoxymethyl)-5-(4-((tert-butoxycarbonyl)(cyclopentyl)amino)-6-chloro-1H-pyrazolo[3,4-b]pyridin-1-yl)tetrahydrofuran-3,4-diyl diacetate (25 g, 42.01 mmol, yield 77.01%) as a light yellow oil.

¹H NMR: 400 MHz, DMSO-d₆; δ=1.34 (s, 9H), 1.38-1.48 (m, 2H), 1.48-1.74 (m, 6H), 1.97 (s, 3H), 2.08 (s, 3H), 2.10-2.12 (m, 3H), 4.04-4.07 (m, 1H), 4.33-4.43 (m, 3H), 5.66 (t, J=5.69 Hz, 1H), 5.88 (dd, J=5.38, 3.38 Hz, 1H), 6.50 (d, J=3.25 Hz, 1H), 7.27 (s, 1H), 8.28 (s, 1H).

### Step 6

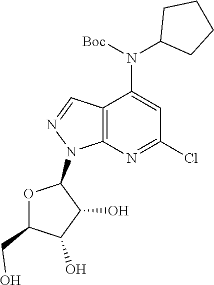 [SMILES: CC(C)(C)OC(=O)N(c1cc(Cl)nc2c1cnn2[C@@H]1O[C@H](CO)[C@@H](O)[C@H]1O)C1CCCC1]

### tert-Butyl (6-chloro-1-((2R,3R,4S,5R)-3,4-dihydroxy-5-(hydroxymethyl)tetrahydrofuran-2-yl)-1H-pyrazolo[3,4-b]pyridin-4-yl)(cyclopentyl)carbamate

A solution of (2R,3R,4R,5R)-2-(acetoxymethyl)-5-(4-((tert-butoxycarbonyl)(cyclopentyl)amino)-6-chloro-1H-pyrazolo[3,4-b]pyridin-1-yl)tetrahydrofuran-3,4-diyl diacetate (25 g, 42.01 mmol, 1 eq) in NH₃/MeOH (7 M, 40 mL) was stirred at 20° C. for 12 h. The reaction mixture was concentrated under reduced pressure to give tert-butyl (6-chloro-1-((2R,3R,4S,5R)-3,4-dihydroxy-5-(hydroxymethyl)tetrahydrofuran-2-yl)-1H-pyrazolo[3,4-b]pyridin-4-yl)(cyclopentyl)carbamate (19 g, 40.52 mmol, yield 96.44%) as a brown-yellow oil.

¹H NMR: 400 MHz, DMSO-d₆; δ=1.34 (s, 9H), 1.45-1.51 (m, 2H), 1.61 (br s, 4H), 1.91-1.98 (m, 2H), 3.39-3.45 (m, 1H), 3.53-3.59 (m, 1H), 3.93 (q, J=4.92 Hz, 1H), 4.21 (q, J=5.00 Hz, 1H), 4.36 (br t, J=8.19 Hz, 1H), 4.66 (q, J=5.13 Hz, 1H), 4.73 (t, J=5.75 Hz, 1H), 5.20 (d, J=5.63 Hz, 1H), 5.42 (d, J=6.00 Hz, 1H), 6.20 (d, J=4.88 Hz, 1H), 7.22 (s, 1H), 8.17 (s, 1H).

### Step 7

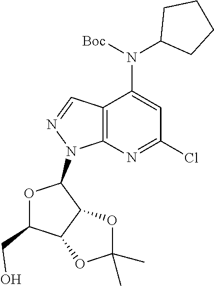 [SMILES: CC(C)(C)OC(=O)N(c1cc(Cl)nc2c1cnn2[C@@H]1O[C@H](CO)[C@H]2OC(C)(C)O[C@H]21)C1CCCC1]

### tert-Butyl (6-chloro-1-((3aR,4R,6R,6aR)-6-(hydroxymethyl)-2,2-dimethyltetrahydrofuro[3,4-d][1,3]dioxol-4-yl)-1H-pyrazolo[3,4-b]pyridin-4-yl)(cyclopentyl)carbamate

To a solution of tert-butyl (6-chloro-1-((2R,3R,4S,5R)-3,4-dihydroxy-5-(hydroxymethyl)tetrahydrofuran-2-yl)-1H-pyrazolo[3,4-b]pyridin-4-yl)(cyclopentyl)carbamate (19 g, 40.52 mmol, 1 eq) in DMF (220 mL) was added 2,2-dimethoxypropane (12.66 g, 121.55 mmol, 14.89 mL, 3 eq) and p-toluenesulfonic acid (1.40 g, 8.10 mmol, 0.2 eq). The mixture was stirred at 70° C. for 1 h. The mixture was diluted with water (2 L) and extracted with ethyl acetate (3×500 mL). The combined organic layer was washed with brine (2500 mL), dried over Na₂SO₄, filtered and concentrated under reduced pressure to give a residue. The residue was purified by column chromatography (SiO₂, Petroleum ether/Ethyl acetate=0:1 to 1:1) to give tert-butyl (6-chloro-1-((3aR,4R,6R,6aR)-6-(hydroxymethyl)-2,2-dimethyltetrahydrofuro[3,4-d][1,3]dioxol-4-yl)-1H-pyrazolo[3,4-b]pyridin-4-yl)(cyclopentyl)carbamate (18 g, 35.36 mmol, yield 87.28%) as a light-yellow oil.

¹H NMR: 400 MHz, DMSO-d₆; δ=1.33 (s, 9H), 1.35 (s, 3H), 1.48 (br d, J=2.88 Hz, 2H), 1.54 (s, 3H), 1.58-1.65 (m, 4H), 1.91-1.98 (m, 2H), 3.26-3.31 (m, 1H), 3.42-3.48 (m, 1H), 4.13-4.17 (m, 1H), 4.37 (quin, J=8.35 Hz, 1H), 4.89 (t, J=5.82 Hz, 1H), 4.97 (dd, J=6.13, 2.13 Hz, 1H), 5.40 (dd, J=6.13, 1.75 Hz, 1H), 6.41 (d, J=1.63 Hz, 1H), 7.24 (s, 1H), 8.21 (s, 1H).

### Step 8

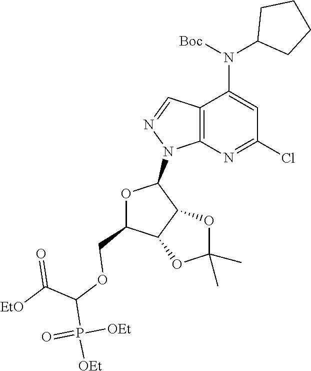 [SMILES: CCOC(=O)C(OC[C@H]1O[C@@H](n2ncc3c(N(C(=O)OC(C)(C)C)C4CCCC4)cc(Cl)nc32)[C@@H]2OC(C)(C)O[C@@H]21)P(=O)(OCC)OCC]

### Ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)acetate

To a solution of tert-butyl (6-chloro-1-((3aR,4R,6R,6aR)-6-(hydroxymethyl)-2,2-dimethyltetrahydrofuro[3,4-d][1,3]dioxol-4-yl)-1H-pyrazolo[3,4-b]pyridin-4-yl)(cyclopentyl)carbamate (13 g, 25.54 mmol, 1 eq) in toluene (130 mL) was added ethyl 2-diazo-2-(diethoxyphosphoryl)acetate (12.78 g, 51.08 mmol, 2 eq) and Rh(OAc)₂ (1.13 g, 5.11 mmol, 0.2 eq) under nitrogen. The mixture was stirred at 95° C. for 1 h. The mixture was diluted with water (200 mL) and extracted with ethyl acetate (3×200 mL). The combined organic layer was washed with brine (600 mL), dried over Na₂SO₄, filtered and concentrated under reduced pressure. The residue was purified by column chromatography (SiO₂, Petroleum ether/Ethyl acetate=0:1 to 1:1) to give ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)acetate (15 g, 20.52 mmol, 80.32% yield) as a light-yellow oil.

LCMS (ESI+): m/z 731.4 [M+H]⁺, Rt: 0.689 min.

### Step 9

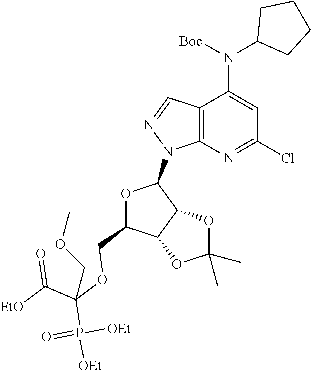 [SMILES: CCOC(=O)C(COC)(OC[C@H]1O[C@@H](n2ncc3c(N(C(=O)OC(C)(C)C)C4CCCC4)cc(Cl)nc32)[C@@H]2OC(C)(C)O[C@@H]21)P(=O)(OCC)OCC]

### Ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoate

To a solution of ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)acetate (13 g, 17.78 mmol, 1 eq) in THF (130 mL) was added sodium bis(trimethylsilyl)amide (1 M, 35.56 mL, 2 eq) dropwise at −78° C. The mixture was stirred at −78° C. for 30 min. To the mixture was added tetrabutylammonium iodide (3.28 g, 8.89 mmol, 0.5 eq) and then to the mixture was added bromo(methoxy)methane (7.78 g, 62.23 mmol, 5.08 mL, 3.5 eq) dropwise. The mixture was stirred and slowly brought to 25° C. for 4 h. The reaction mixture was quenched by addition of NH₄Cl (35 mL) at 0° C., and then diluted with water (200 mL) and extracted with ethyl acetate (3×200 mL). The combined organic layer was washed with brine (600 mL), dried over Na₂SO₄, filtered and concentrated under reduced pressure. The residue was purified by column chromatography (SiO₂, Petroleum ether/Ethyl acetate=0:1 to 1:1) to give ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoate (12 g, 15.48 mmol, yield 87.06%) as a light-yellow oil.

¹H NMR: 400 MHz, DMSO-d₆; δ=1.28-1.32 (m, 6H), 1.38 (s, 9H), 1.54 (br d, J=5.13 Hz, 2H), 1.61 (s, 9H), 1.66 (br dd, J=10.13, 5.25 Hz, 4H), 1.91-1.99 (m, 2H), 3.35-3.39 (m, 2H), 3.81-3.91 (m, 2H), 3.95-4.04 (m, 2H), 4.13-4.32 (m, 7H), 4.40 (br t, J=8.38 Hz, 1H), 4.48 (td, J=5.38, 2.63 Hz, 1H), 5.22 (td, J=6.47, 2.44 Hz, 1H), 5.43 (dt, J=6.00, 2.50 Hz, 1H), 6.66 (dd, J=6.75, 1.88 Hz, 1H), 6.91 (s, 1H), 7.85 (d, J=1.50 Hz, 1H).

### Step 10

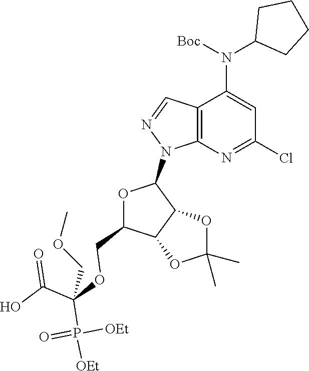 [SMILES: CCOP(=O)(OCC)[C@](COC)(OC[C@H]1O[C@@H](n2ncc3c(N(C(=O)OC(C)(C)C)C4CCCC4)cc(Cl)nc32)[C@@H]2OC(C)(C)O[C@@H]21)C(=O)O]

### (S)-2-(((3aR,4R,6R,6aR)-6-(4-((tert-Butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoic Acid

To a solution of ethyl 2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoate (9 g, 11.61 mmol, 1 eq) in THF (504 mL) and H₂O (252 mL) was added LiOH·H₂O (730.71 mg, 17.41 mmol, 36 mL, 1.5 eq). The mixture was stirred at 40° C. for 36 h. The mixture was acidified to pH 6 by slow addition of citric acid. The reaction mixture was concentrated under reduced pressure to remove THF. The residue was diluted with water (500 mL) and extracted with ethyl acetate (3×200 mL). The combined organic layer was washed with brine (600 mL), dried over Na₂SO₄, filtered and concentrated under reduced pressure. The residue was purified by prep-HPLC (column: Agela DuraShell C18 250*70 mm*10 um; mobile phase: [water(NH₄HCO₃)-ACN]; B %: 42%-62%, 20 min) to give desired compound (4.2 g, yield 26%, purity 90%) as a light yellow oil, which was further separated by SFC (column: DAICEL CHIRALPAK IC(250 mm*30 mm, 10 um); mobile phase: [0.1% NH₃H₂O ETOH]; B %: 40%-40%, 10 min) to give (S)-2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoic acid (1.9 g, 2.54 mmol, 21.90% yield) as a light yellow oil.

¹H NMR: 400 MHz, MeOD; δ=1.25 (d, J=7.15 Hz, 6H), 1.40 (s, 12H), 1.55-1.63 (m, 5H), 1.64-1.74 (m, 4H), 1.97-2.01 (m, 1H), 2.04 (br s, 1H), 3.31-3.31 (m, 3H), 3.73-3.80 (m, 2H), 3.87-3.99 (m, 2H), 4.11-4.20 (m, 4H), 4.40-4.49 (m, 2H), 5.22 (dd, J=6.02, 2.01 Hz, 1H), 5.47 (dd, J=6.02, 1.51 Hz, 1H), 6.57 (d, J=1.13 Hz, 1H), 7.12 (s, 1H), 8.04 (s, 1H).

### Step 11

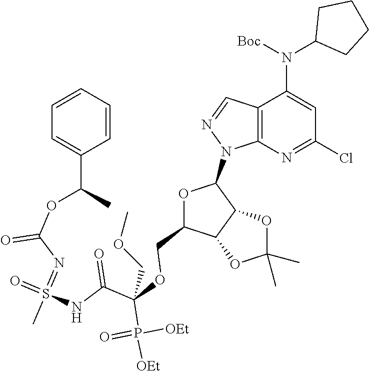 [SMILES: CCOP(=O)(OCC)[C@](COC)(OC[C@H]1O[C@@H](n2ncc3c(N(C(=O)OC(C)(C)C)C4CCCC4)cc(Cl)nc32)[C@@H]2OC(C)(C)O[C@@H]21)C(=O)N[S@@](C)(=O)=NC(=O)O[C@H](C)c1ccccc1]

### tert-Butyl (6-chloro-1-((3aR,4R,6R,6aR)-6-((((S)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate

To a solution of NaH (64.24 mg, 1.61 mmol, 60% purity, 3 eq) in DMF (2 mL) was added (R)-1-phenylethyl (amino(methyl)(oxo)-λ⁶-sulfaneylidene)carbamate (168.63 mg, 695.96 μmol, 1.3 eq). The mixture was stirred at 25° C. for 0.5 h. To a mixture of (S)-2-(((3aR,4R,6R,6aR)-6-(4-((tert-butoxycarbonyl)(cyclopentyl)amino)-6-chloro-1H-pyrazolo[3,4-b]pyridin-1-yl)-2,2-dimethyltetrahydrofuro[3,4-d][1,3]dioxol-4-yl)methoxy)-2-(diethoxyphosphoryl)-3-methoxypropanoic acid (400 mg, 535.35 μmol, 1 eq) in DMF (2 mL) was added HATU (223.91 mg, 588.89 μmol, 1.1 eq) and 4-methylmorpholine (162.45 mg, 1.61 mmol, 176.57 μL, 3 eq). The mixture was stirred at 25° C. for 0.5 h. Then the mixture was added the above solution at 25° C. and stirred for 1 h. The reaction mixture was quenched by addition saturated NH₄Cl aqueous solution (5 mL) at 0° C., and then diluted with water (20 mL) and extracted with ethyl acetate (3×20 mL). The combined organic layers were washed with brine (50 mL), dried over Na₂SO₄, filtered and concentrated under reduced pressure to give tert-butyl (6-chloro-1-((3aR,4R,6R,6aR)-6-((((S)-2-(diethoxyphosphoryl)-3-methoxy-1-oxo-1-((S)—N—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate (240 mg, 247.05 μmol, yield 46.15%) as a light-yellow oil.

LCMS: m/z 971.5 [M+H]⁺, Rt: 0.702 min.

### Step 12

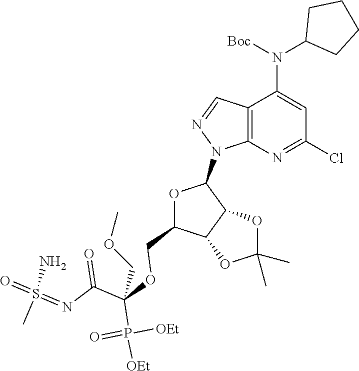 [SMILES: CCOP(=O)(OCC)[C@](COC)(OC[C@H]1O[C@@H](n2ncc3c(N(C(=O)OC(C)(C)C)C4CCCC4)cc(Cl)nc32)[C@@H]2OC(C)(C)O[C@@H]21)C(=O)N=[S@](C)(N)=O]

### tert-Butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-b]pyridin-4-yl)(cyclopentyl)carbamate

To a solution of tert-butyl (6-chloro-1-((3aR,4R,6R,6aR)-6-((((S)-2-(diethoxyphosphoryl)-3-methoxy-1-oxo-1-((S)—N—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate (110 mg, 113.23 μmol, 1 eq) in THF (2 mL) was added PtO₂ (25.71 mg, 113.23 μmol, 1 eq) and CHCl₃ (40.55 mg, 339.70 μmol, 27.40 μL, 3 eq) under H₂. The mixture was stirred at 25° C. for 2 h. Two additional vials were set up as described above. All three reaction mixtures were combined for purification. The reaction mixture was filtered and concentrated under reduced pressure. The residue was purified by prep-TLC (SiO₂, Ethyl acetate:Methanol=20:1) to give tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-b]pyridin-4-yl)(cyclopentyl)carbamate (210 mg, 255.07 μmol, yield 75.09%) as a colorless oil.

¹H NMR: 400 MHz, CDCl₃; δ=1.24-1.29 (m, 6H), 1.40 (s, 12H), 1.56-1.62 (m, 5H), 1.65-1.73 (m, 4H), 1.98-2.05 (m, 2H), 3.31 (s, 3H), 3.34 (s, 3H), 3.69 (dd, J=9.05, 6.11 Hz, 1H), 3.82-3.86 (m, 1H), 3.86-3.95 (m, 1H), 4.12-4.21 (m, 4H), 4.45-4.48 (m, 1H), 5.35 (dd, J=6.05, 1.65 Hz, 1H), 5.48-5.51 (m, 1H), 6.56 (s, 1H), 7.12 (s, 1H), 8.04 (s, 1H).

### Step 13

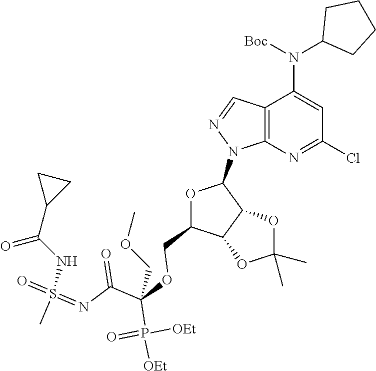 [SMILES: CCOP(=O)(OCC)[C@](COC)(OC[C@H]1O[C@@H](n2ncc3c(N(C(=O)OC(C)(C)C)C4CCCC4)cc(Cl)nc32)[C@@H]2OC(C)(C)O[C@@H]21)C(=O)N=[S@@](C)(=O)NC(=O)C1CC1]

### tert-Butyl (6-chloro-1-((3aR,4R,6R,6aR)-6-((((S)-1-(((R)-cyclopropanecarboxamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate

To a solution of tert-butyl (1-((3aR,4R,6R,6aR)-6-((((S)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-b]pyridin-4-yl)(cyclopentyl)carbamate (50.00 mg, 60.73 μmol, 1 eq) in dichloromethane (1 mL) was added triethylamine (18.44 mg, 182.20 μmol, 25.36 μL, 3 eq) and cyclopropanecarbonyl chloride (12.70 mg, 121.46 μmol, 11.04 μL, 2 eq). The mixture was stirred at 25° C. for 2 h. The reaction mixture was quenched by addition water at 0° C., and then diluted with water (3 mL) and extracted with ethyl acetate (3×2 mL). The combined organic layers were washed with brine (6 mL), dried over Na₂SO₄, filtered and concentrated under reduced pressure to give tert-butyl (6-chloro-1-((3aR,4R,6R,6aR)-6-((((S)-1-(((R)-cyclopropanecarboxamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate (45 mg, 50.48 μmol, yield 83.13%) as a colorless oil.

LCMS: m/z 891.4 [M+H]⁺, Rt: 0.659 min.

### Step 14

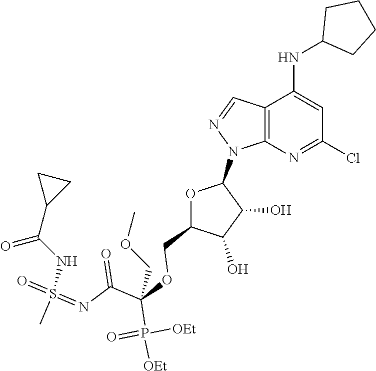 [SMILES: CCOP(=O)(OCC)[C@](COC)(OC[C@H]1O[C@@H](n2ncc3c(NC4CCCC4)cc(Cl)nc32)[C@H](O)[C@@H]1O)C(=O)N=[S@@](C)(=O)NC(=O)C1CC1]

### Diethyl ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonate

To a solution of tert-butyl (6-chloro-1-((3aR,4R,6R,6aR)-6-((((S)-1-(((R)-cyclopropanecarboxamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-1H-pyrazolo[3,4-b]pyridin-4-yl)(cyclopentyl)carbamate (45 mg, 50.48 μmol, 1 eq) in dichloromethane (1 mL) was added H₂O (1.82 mg, 100.97 μmol, 1.82 μL, 2 eq) and trifluoroacetic acid (0.2 mL). The mixture was stirred at 25° C. for 10 min. The reaction mixture was concentrated under reduced pressure to give a residue. To the residue was added ethyl acetate (1 mL) and triethylamine (0.05 mL). The mixture was concentrated under reduced pressure to give diethyl ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-_i-sulfaneylidene)amino)-3-methoxy-1-oxopropan-2-yl)phosphonate (35 mg, 46.59 μmol, yield 92.29%) as a light-yellow solid.

LCMS (ESI+): m/z 751.3 [M+H]⁺, Rt: 0.459 min.

### Step 15

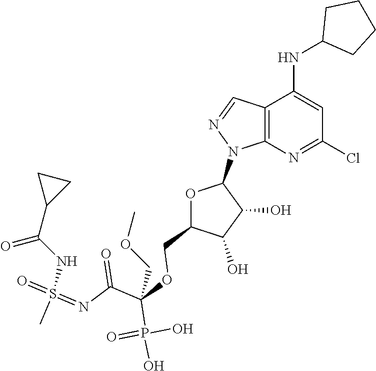 [SMILES: COC[C@@](OC[C@H]1O[C@@H](n2ncc3c(NC4CCCC4)cc(Cl)nc32)[C@H](O)[C@@H]1O)(C(=O)N=[S@@](C)(=O)NC(=O)C1CC1)P(=O)(O)O]

### ((S)-2-(((2R,3S,4R,5R)-5-(6-Chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonic Acid

To a solution of diethyl ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonate (35 mg, 46.59 μmol, 1 eq) in dichloromethane (1 mL) was added TEA (94.29 mg, 931.86 μmol, 129.70 μL, 20 eq) and TMSI (139.84 mg, 698.90 μmol, 95.13 μL, 15 eq). The mixture was stirred at 25° C. for 1 h. The reaction mixture was concentrated under reduced pressure. The residue was purified by prep-HPLC (column: Waters XBridge BEH C18 100*30 mm*10 um; mobile phase: [water (NH₃H₂O+NH₄HCO₃)-ACN]; B %: 5%-35%, 8 min) to give ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphonic acid (11.5 mg, 16.40 μmol, yield 35.19%, purity 99.1%) as a white solid.

¹H NMR: 400 MHz, MeOD-d₄; δ=0.67-0.80 (m, 2H), 0.84 (br d, J=4.13 Hz, 2H), 1.44-1.56 (m, 1H), 1.62-1.73 (m, 4H), 1.76-1.85 (m, 2H), 2.06-2.15 (m, 2H), 3.33 (br s, 3H), 3.39 (s, 3H), 3.92 (dd, J=11.38, 2.88 Hz, 1H), 3.98-4.05 (m, 1H), 4.10 (m, 2H), 4.15-4.21 (m, 1H), 4.23-4.27 (m, 1H), 4.60-4.64 (m, 1H), 4.77-4.79 (m, 1H), 6.26 (s, 1H), 6.31 (d, J=4.50 Hz, 1H), 8.20 (s, 1H).

LCMS (ESI+): m/z 695.2 [M+H]⁺, Rt: 2.122 min.

### Example 20

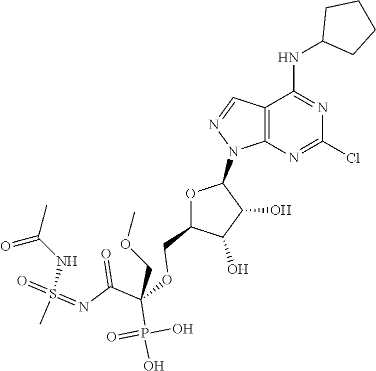 [SMILES: COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

### ((R)-1-(((R)-acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

### Step 1

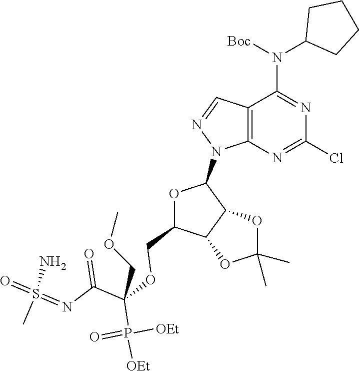 [SMILES: CCOP(=O)(OCC)[C@@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@](C)(N)=O]

### Tert-butyl (1-((3aR,4R,6R,6aR)-6-((((R)-1-(((S)-amino(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a mixture of tert-butyl (6-chloro-1-((3aR,4R,6R,6aR)-6-((((R)-2-(diethoxyphosphoryl)-3-methoxy-1-oxo-1-((S)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (120 mg, 123.40 μmol, 1 eq) in EtOH (2 mL) was added PtO₂ (28.02 mg, 123.40 μmol, 1 eq) and CHCl₃ (44.19 mg, 370.20 μmol, 29.86 μL, 3 eq), the resulting mixture was stirred at 20° C. under H₂ (15 Psi) for 12 h. The mixture was filtered and concentrated in vacuum. The residue was purified by prep-TLC (EtOAc/MeOH=20/1) to give the title compound (80 mg, yield 78.65%) as a colorless oil.

¹H NMR: ET54743-405-P1C, 400 MHz, MeOD-d₄. 6=1.24 (s, 3H), 1.28-1.31 (m, 3H), 1.40 (s, 3H), 1.56 (s, 9H), 1.58-1.65 (m, 5H), 1.85-2.00 (m, 4H), 2.02-2.09 (m, 2H), 3.33 (s, 3H), 3.40 (s, 3H), 3.69 (dd, J=9.01, 5.50 Hz, 1H), 3.83-3.92 (m, 1H), 3.92-3.98 (m, 2H), 4.12-4.24 (m, 4H), 4.41-4.48 (m, 1H), 4.59 (s, 2H), 5.03 (t, J=8.69 Hz, 1H), 5.34 (dd, J=6.07, 1.94 Hz, 1H), 5.46 (dd, J=6.00, 1.25 Hz, 1H), 6.48 (s, 1H), 8.07 (s, 1H).

### Step 2

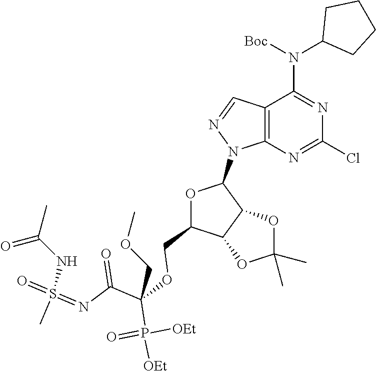 [SMILES: CCOP(=O)(OCC)[C@@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N=[S@@](C)(=O)NC(C)=O]

### Tert-butyl (1-((3aR,4R,6R,6aR)-6-((((R)-1-(((R)-acetamido(methyl)(oxo)-λ⁶-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate

To a mixture of tert-butyl (1-((3aR,4R,6R,6aR)-6-((((R)-1-(((S)-amino(methyl)(oxo)-16-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (40 mg, 48.53 μmol, 1 eq) in DCM (1 mL) was added TEA (9.82 mg, 97.05 μmol, 13.51 μL, 2 eq), DMAP (592.85 ug, 4.85 μmol, 0.1 eq) and Ac₂O (5.45 mg, 53.38 μmol, 5.00 μL, 1.1 eq), the resulting mixture was stirred at 20° C. for 1 h. The mixture was poured into water (5 mL), the aqueous layer was extracted with DCM (3×3 mL), the combined organic layer was washed with brine (2×3 mL), the combined organic layer was dried with anhydrous Na₂SO₄, filtered and concentrated in vacuum to give the title compound (40 mg, yield 95.15%) as a colorless oil.

LCMS (ESI+): m/z 865.9 [M+H]⁺, Rt:0.893 min.

### Step 3

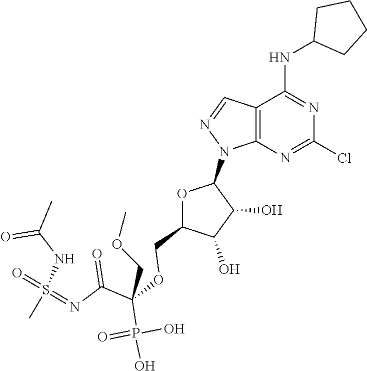 [SMILES: COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

### ((R)-1-(((R)-acetamido(methyl)(oxo)-λ⁶-sulfaneylidene)amino)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-oxopropan-2-yl)phosphonic Acid

To a mixture of tert-butyl (1-((3aR,4R,6R,6aR)-6-((((R)-1-(((R)-acetamido(methyl)(oxo)-16-sulfaneylidene)amino)-2-(diethoxyphosphoryl)-3-methoxy-1-oxopropan-2-yl)oxy)methyl)-2,2-dimethyltetrahydrofuro[3,4-d][1,3]dioxol-4-yl)-6-chloro-1H-pyrazolo[3,4-d]pyrimidin-4-yl)(cyclopentyl)carbamate (40 mg, 46.17 μmol, 1 eq) in DCM (1 mL) was added H₂O (1.66 mg, 92.35 μmol, 1.66 μL, 2 eq) and TFA (154.00 mg, 1.35 mmol, 100.00 μL, 29.25 eq), the resulting mixture was stirred at 20° C. for 1 h. The mixture was filtered and concentrated under reduced pressure. The residue was added TEA (83.73 mg, 827.42 μmol, 115.17 μL, 20 eq) and TMSI (124.17 mg, 620.56 μmol, 84.47 μL, 15 eq) in DCM (1 mL), the resulting mixture was stirred at 20° C. for 1 h. The mixture was filtered and concentrated under reduced pressure and the residue was purified by prep-HPLC (column: Waters XBridge BEH C18 100*30 mm*10 um; mobile layers: [water (NH3H2O+NH4HCO3)-ACN]; B %: 1%-30%, 8 min) to give the title compound (11 mg, 16.29 μmol, 39.37% yield, 99.2% purity) as a white solid.

¹H NMR: HNMR, 400 MHz, CD₃OD. 6=1.51-1.74 (m, 4H), 1.76-1.87 (m, 2H), 1.99 (s, 3H), 2.04-2.16 (m, 2H), 3.32 (br s, 3H), 3.43 (s, 3H), 3.91 (dd, J=10.69, 2.81 Hz, 1H), 4.02-4.13 (m, 2H), 4.17-4.28 (m, 2H), 4.52-4.57 (m, 1H), 4.68 (t, J=4.88 Hz, 1H), 4.88-4.88 (m, 1H), 6.21 (d, J=4.50 Hz, 1H), 8.14 (s, 1H).

LCMS (ESI+): m/z 670.3 [M+H]⁺, Rt:1.385 min.

The following compounds can generally be made using the methods described above. It is expected that these compounds when made will have activity similar to those that have been made in the examples disclosed herein.

### Example 21

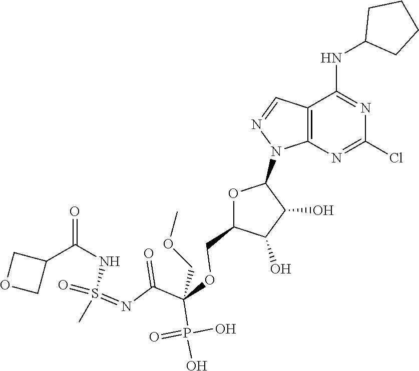 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1COC1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxetane-3-carboxamido)(oxo)-λ⁶-sulfaneylidene)amino)-1-oxopropan-2-yl)phosphonic Acid

¹H NMR: HNMR, 400 MHz, CD₃OD. δ=1.55-1.73 (m, 4H), 1.77-1.85 (m, 2H), 2.05-2.14 (m, 2H), 3.34 (m, 4H), 3.55 (s, 3H), 3.95 (dd, J=11.26, 2.75 Hz, 1H), 4.00 (dd, J=10.13, 4.75 Hz, 1H), 4.10-4.15 (m, 1H), 4.16-4.19 (m, 2H), 4.20-4.25 (m, 1H), 4.26-4.29 (m, 1H), 4.51-4.56 (m, 1H), 4.62-4.65 (m, 1H), 4.80 (br s, 1H), 5.63 (d, J=1.00 Hz, 1H), 6.06 (s, 1H), 6.23 (d, J=4.75 Hz, 1H), 8.16 (s, 1H).

LCMS (ESI+): m/z 712.2 [M+H]⁺, Rt:1.897 min.

### Example 22

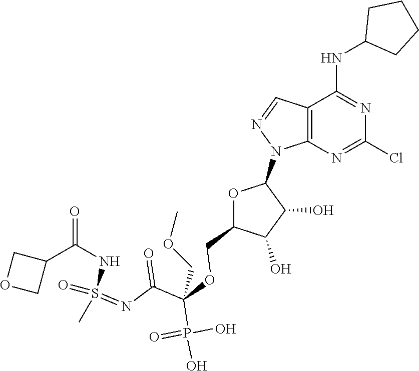 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1COC1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(oxetane-3-carboxamido)(oxo)-λ⁶-sulfaneylidene)amino)-1-oxopropan-2-yl)phosphonic Acid

¹H NMR: 400 MHz, CD₃OD. δ=1.57-1.71 (m, 4H), 1.81 (br d, J=5.25 Hz, 2H), 2.06-2.14 (m, 2H), 3.32 (br s, 3H), 3.34-3.35 (m, 1H), 3.53 (s, 3H), 3.92-3.98 (m, 1H), 4.02-4.08 (m, 1H), 4.11-4.16 (m, 1H), 4.18-4.26 (m, 4H), 4.52-4.56 (m, 1H), 4.67 (br d, J=4.38 Hz, 1H), 4.76 (br s, 1H), 5.68 (s, 1H), 6.11 (s, 1H), 6.23 (d, J=4.50 Hz, 1H), 8.19 (s, 1H).

LCMS (ESI-): m/z 710.1 [M−H]⁻, Rt:1.913 min.

### Example 23

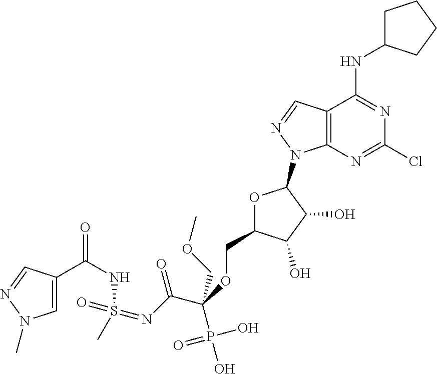 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cnn(C)c1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(1-methyl-1H-pyrazole-4-carboxamido)(oxo)-λ⁶-sulfaneylidene)amino)-1-oxopropan-2-yl)phosphonic Acid

¹H NMR: HNMR, 400 MHz, CD₃OD. δ=1.54-1.72 (m, 4H), 1.76-1.83 (m, 2H), 2.03-2.13 (m, 2H), 3.35 (s, 3H), 3.65 (s, 3H), 3.85 (s, 1H), 4.94-4.03 (m, 2H), 4.13-4.18 (m, 1H), 4.25-4.31 (m, 2H), 4.44-4.48 (m, 1H), 4.58-4.62 (m, 1H), 4.84 (br s, 1H), 6.24 (d, J=4.75 Hz, 1H), 7.58 (s, 1H), 7.71 (s, 1H), 8.14 (s, 1H).

LCMS (ESI+): m/z 736.2 [M+H]⁺, Rt:1.941 min.

### Example 24

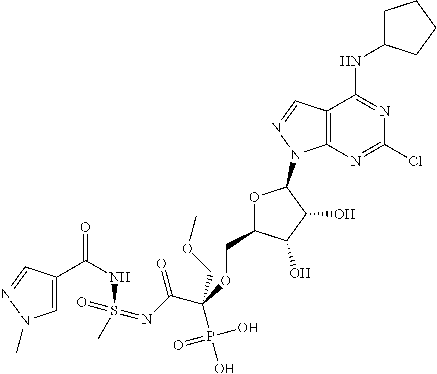 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1cnn(C)c1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(1-methyl-1H-pyrazole-4-carboxamido)(oxo)-λ⁶-sulfaneylidene)amino)-1-oxopropan-2-yl)phosphonic Acid

¹H NMR: HNMR, 400 MHz, CD₃OD. 6=1.57-1.70 (m, 4H), 1.76-1.83 (m, 2H), 2.05-2.13 (m, 2H), 3.32 (br s, 3H), 3.53 (s, 3H), 3.87 (s, 3H), 3.90-3.95 (m, 1H), 4.05-4.15 (m, 2H), 4.17-4.23 (m, 2H), 4.25-4.30 (m, 1H), 4.50-4.56 (m, 1H), 4.60-4.65 (m, 1H), 4.76 (br s, 1H), 6.23 (d, J=4.50 Hz, 1H), 7.77 (s, 1H), 7.92 (s, 1H), 8.15 (s, 1H).

LCMS (ESI+): m/z 736.2 [M+H]⁺, Rt:1.931 min.

### Example 25

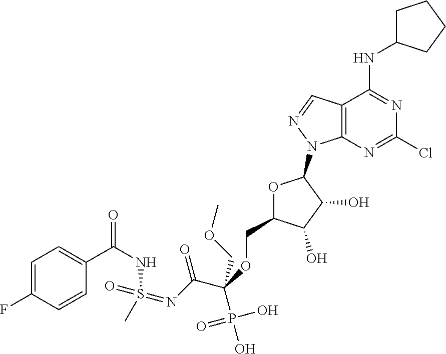 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1ccc(F)cc1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-1-(((4-fluorobenzamido)(methyl)(oxo)-λ⁶-sulfaneylidene)amino)-3-methoxy-1-oxopropan-2-yl)phosphonic Acid

¹H NMR: HNMR, 400 MHz, MeOD-d₄. 6=1.58 (dt, J=12.51, 6.38 Hz, 2H), 1.63-1.73 (m, 2H), 1.75-1.84 (m, 2H), 2.02-2.13 (m, 2H), 3.34 (s, 3H), 3.55 (br s, 3H), 3.96 (dd, J=10.94, 2.94 Hz, 1H), 4.11 (br d, J=3.63 Hz, 2H), 4.22 (br dd, J=10.63, 5.50 Hz, 1H), 4.28 (q, J=4.29 Hz, 1H), 4.45-4.52 (m, 1H), 4.62 (t, J=4.88 Hz, 1H), 4.76 (br s, 1H), 6.21 (d, J=4.63 Hz, 1H), 7.01 (t, J=8.76 Hz, 2H), 7.92 (br s, 2H), 8.11 (s, 1H).

LCMS (ESI+): m/z 750.1 [M+H]⁺, Rt: 2.171 min.

### Example 26

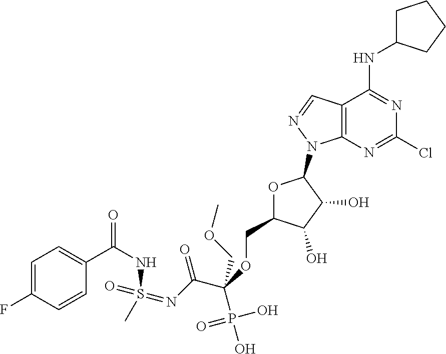 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1ccc(F)cc1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-1-(((4-fluorobenzamido)(methyl)(oxo)-λ⁶-sulfaneylidene)amino)-3-methoxy-1-oxopropan-2-yl)phosphonic Acid

¹H NMR: 400 MHz, CD₃OD. δ=1.52-1.62 (m, 2H), 1.63-1.70 (m, 2H), 1.75-1.83 (m, 2H), 2.03-2.11 (m, 2H), 3.32 (br s, 3H), 3.61 (s, 3H), 3.93-4.00 (m, 1H), 4.06 (dd, J=10.44, 4.06 Hz, 1H), 4.13-4.18 (m, 1H), 4.21-4.29 (m, 2H), 4.49 (t, J=6.94 Hz, 1H), 4.66 (t, J=4.50 Hz, 1H), 4.82 (br s, 1H), 6.23 (d, J=4.88 Hz, 1H), 7.04 (t, J=8.69 Hz, 2H), 7.93 (br dd, J=7.44, 5.82 Hz, 2H), 8.16 (s, 1H).

LCMS (ESI+): m/z 750.2 [M+H]⁺, Rt:2.167 min.

### Example 27

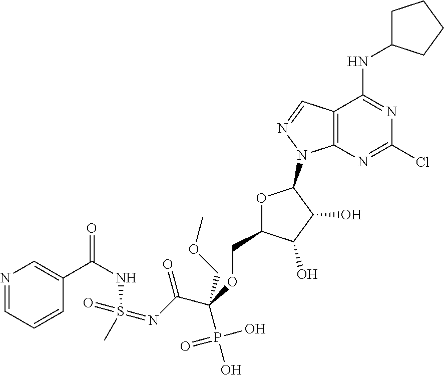 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cccnc1)P(=O)(O)O]

### ((2S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-3-methoxy-1-((methyl(nicotinamido)(oxo)-λ⁶-sulfaneylidene)amino)-1-oxopropan-2-yl)phosphonic Acid

¹H NMR: HNMR, 400 MHz, CD₃OD. δ=1.49-1.62 (m, 2H), 1.62-1.71 (m, 2H), 1.73-1.83 (m, 2H), 2.01-2.11 (m, 2H), 3.35 (s, 3H), 3.70 (br s, 3H), 3.95-4.00 (m, 1H), 4.01-4.07 (m, 1H), 4.13-4.19 (m, 1H), 4.26-4.32 (m, 2H), 4.39-4.44 (m, 1H), 4.61 (br s, 1H), 4.79-4.80 (m, 1H), 6.22 (d, J=5.63 Hz, 1H), 7.32-7.37 (m, 1H), 8.11 (s, 2H), 8.56-8.60 (m, 1H), 8.94 (s, 1H).

LCMS (ESI+): m/z 733.1 [M+H]⁺, Rt:1.988 min.

### Example 28

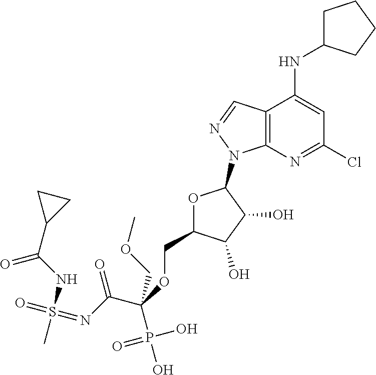 [SMILES: COC[C@@](OC[C@H]1O[C@@H](n2ncc3c(NC4CCCC4)cc(Cl)nc32)[C@H](O)[C@@H]1O)(C(=O)N=[S@](C)(=O)NC(=O)C1CC1)P(=O)(O)O]

### ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-(((S)-cyclopropanecarboxamido(methyl)(oxo)-λ⁶-sulfaneylidene)amino)-3-methoxy-1-oxopropan-2-yl)phosphonic Acid

¹H NMR: 400 MHz, MeOD-d₄. 6=0.74-0.82 (m, 2H), 0.89 (br d, J=3.67 Hz, 2H), 1.55-1.73 (m, 5H), 1.75-1.85 (m, 2H), 2.05-2.15 (m, 2H), 3.34 (s, 3H), 3.47 (s, 3H), 3.92 (br dd, J=11.25, 2.45 Hz, 1H), 3.99-4.06 (m, 1H), 4.07-4.16 (m, 2H), 4.18-4.27 (m, 2H), 4.64 (br t, J=5.32 Hz, 1H), 4.79 (br s, 1H), 6.25 (s, 1H), 6.30 (d, J=4.16 Hz, 1H), 8.20 (s, 1H).

LCMS (ESI+): m/z 695.1 [M+H]⁺, Rt: 1.537 min.

### Example 29

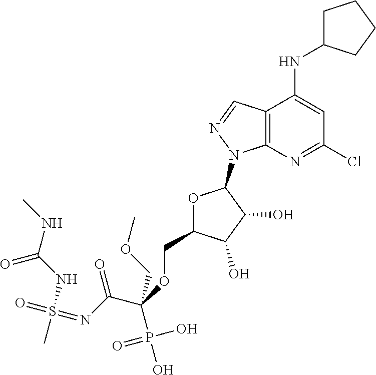 [SMILES: CNC(=O)N[S@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O]

### ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-3-methoxy-1-(((R)-methyl(3-methylureido)(oxo)-λ⁶-sulfaneylidene)amino)-1-oxopropan-2-yl)phosphonic Acid

¹H NMR: HNMR, 400 MHz, MeOD-d₄. 6=1.60-1.74 (m, 4H), 1.75-1.86 (m, 2H), 2.04-2.15 (m, 2H), 2.64 (s, 3H), 3.32-3.32 (m, 3H), 3.32-3.33 (m, 3H), 3.90-3.97 (m, 1H), 4.00-4.05 (m, 1H), 4.06-4.11 (m, 2H), 4.14-4.20 (m, 1H), 4.22-4.28 (m, 1H), 4.67 (br d, J=5.63 Hz, 1H), 4.73 (br s, 1H), 6.25 (s, 1H), 6.28-6.31 (m, 1H), 8.19 (s, 1H).

LCMS (ESI-): m/z 682.1 [M−H]⁻, Rt:2.084 min.

### Example 30

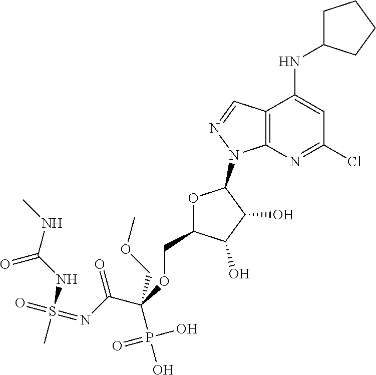 [SMILES: CNC(=O)N[S@@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O]

### ((S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-3-methoxy-1-(((S)-methyl(3-methylureido)(oxo)-λ⁶-sulfaneylidene)amino)-1-oxopropan-2-yl)phosphonic Acid

¹H NMR: 400 MHz, MeOD-d₄. δ=1.62-1.74 (m, 4H), 1.76-1.85 (m, 2H), 2.05-2.15 (m, 2H), 2.66 (s, 3H), 3.33 (s, 3H), 3.39 (s, 3H), 3.93 (dd, J=10.94, 3.31 Hz, 1H), 3.99-4.06 (m, 1H), 4.09 (d, J=4.75 Hz, 2H), 4.18 (dd, J=10.82, 5.32 Hz, 1H), 4.25 (q, J=5.09 Hz, 1H), 4.64 (t, J=5.25 Hz, 1H), 4.77 (t, J=4.69 Hz, 1H), 6.26 (s, 1H), 6.30 (d, J=4.00 Hz, 1H), 8.20 (s, 1H).

LCMS (ESI+): m/z 681.9 [M−H]⁻, Rt: 1.352 min.

### Example 31

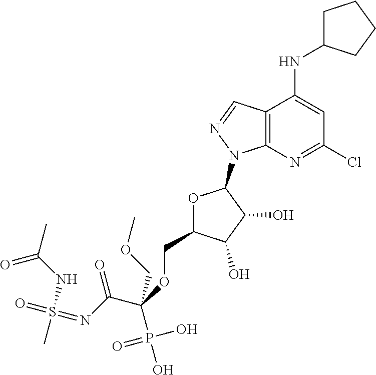 [SMILES: COC[C@@](OC[C@H]1O[C@@H](n2ncc3c(NC4CCCC4)cc(Cl)nc32)[C@H](O)[C@@H]1O)(C(=O)N=[S@@](C)(=O)NC(C)=O)P(=O)(O)O]

### ((S)-1-(((R)-acetamido(methyl)(oxo)-λ⁶-sulfaneylidene)amino)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-3-methoxy-1-oxopropan-2-yl)phosphonic Acid

¹H NMR: 400 MHz, MeOD-d₄. δ=1.61-1.75 (m, 4H), 1.75-1.86 (m, 2H), 1.95 (s, 3H), 2.04-2.15 (m, 2H), 3.32 (s, 3H), 3.36 (s, 3H), 3.91 (dd, J=10.82, 3.31 Hz, 1H), 3.99-4.10 (m, 2H), 4.10-4.19 (m, 2H), 4.24 (q, J=5.13 Hz, 1H), 4.63 (t, J=5.38 Hz, 1H), 4.73-4.77 (m, 1H), 6.26 (s, 1H), 6.29 (d, J=4.00 Hz, 1H), 8.19 (s, 1H).

LCMS (ESI+): m/z 669.2 [M+H]⁺, Rt: 2.064 min.

### Example 32

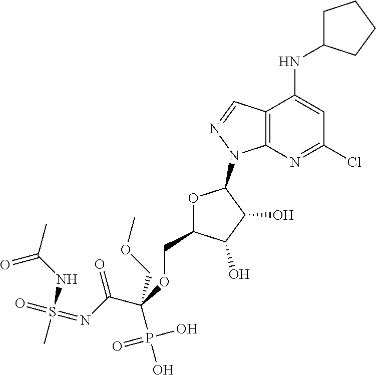 [SMILES: COC[C@@](OC[C@H]1O[C@@H](n2ncc3c(NC4CCCC4)cc(Cl)nc32)[C@H](O)[C@@H]1O)(C(=O)N=[S@](C)(=O)NC(C)=O)P(=O)(O)O]

### ((S)-1-(((S)-acetamido(methyl)(oxo)-λ⁶-sulfaneylidene)amino)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-3-methoxy-1-oxopropan-2-yl)phosphonic Acid

¹H NMR: HNMR, 400 MHz, MeOD-d₄. δ=1.68 (br dd, J=7.40, 4.39 Hz, 4H), 1.78-1.83 (m, 2H), 2.03 (s, 3H), 2.07-2.12 (m, 2H), 3.34 (s, 3H), 3.57 (s, 3H), 3.90 (dd, J=11.04, 2.89 Hz, 1H), 4.00-4.05 (m, 1H), 4.08-4.16 (m, 2H), 4.24 (d, J=4.77 Hz, 2H), 4.66 (t, J=4.89 Hz, 1H), 4.80-4.83 (m, 1H), 6.25 (s, 1H), 6.32 (d, J=4.27 Hz, 1H), 8.20 (s, 1H).

LCMS (ESI+): m/z 669.1 [M+H]⁺, Rt:1.481 min.

### Example 33

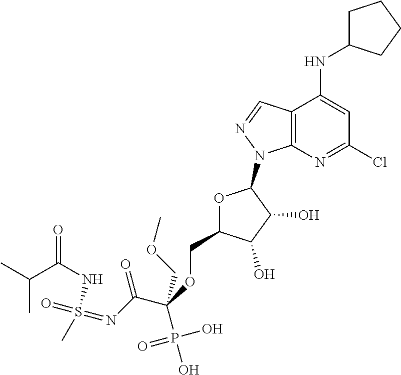 [SMILES: COC[C@@](OC[C@H]1O[C@@H](n2ncc3c(NC4CCCC4)cc(Cl)nc32)[C@H](O)[C@@H]1O)(C(=O)N=[S@@](C)(=O)NC(=O)C(C)C)P(=O)(O)O]

### ((2S)-2-(((2R,3S,4R,5R)-5-(6-chloro-4-(cyclopentylamino)-1H-pyrazolo[3,4-b]pyridin-1-yl)-3,4-dihydroxytetrahydrofuran-2-yl)methoxy)-1-((isobutyramido(methyl)(oxo)-λ⁶-sulfaneylidene)amino)-3-methoxy-1-oxopropan-2-yl)phosphonic Acid

¹H NMR: 400 MHz, MeOD-d₄. δ=1.04 (dd, J=8.69, 6.94 Hz, 6H), 1.63-1.72 (m, 4H), 1.77-1.84 (m, 2H), 2.05-2.13 (m, 2H), 2.25-2.36 (m, 1H), 3.34 (s, 3H), 3.44 (s, 3H), 3.92 (dd, J=10.94, 3.06 Hz, 1H), 3.99-4.05 (m, 1H), 4.10 (d, J=4.75 Hz, 2H), 4.20-4.27 (m, 2H), 4.62 (t, J=4.88 Hz, 1H), 4.80 (br s, 1H), 6.26 (s, 1H), 6.31 (d, J=4.75 Hz, 1H), 8.20 (s, 1H).

LCMS (ESI+): m/z 697.2 [M+H]⁺, Rt: 2.140 min.

### Example 34

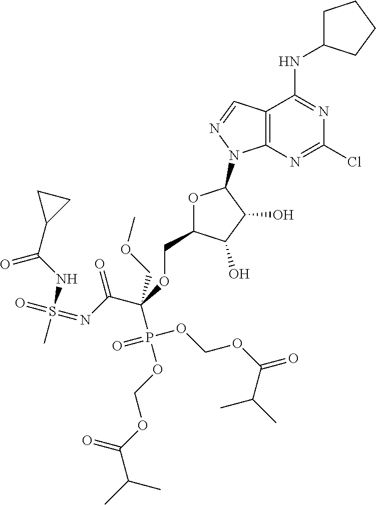 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(OCOC(=O)C(C)C)OCOC(=O)C(C)C]

### ((((S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-1-(((S)-cyclopropanecarboxamido(methyl)(oxo)-λ⁶-sulfaneylidene)amino)-3-methoxy-1-oxopropan-2-yl)phosphoryl)bis(oxy))bis(methylene)bis(2-methylpropanoate)

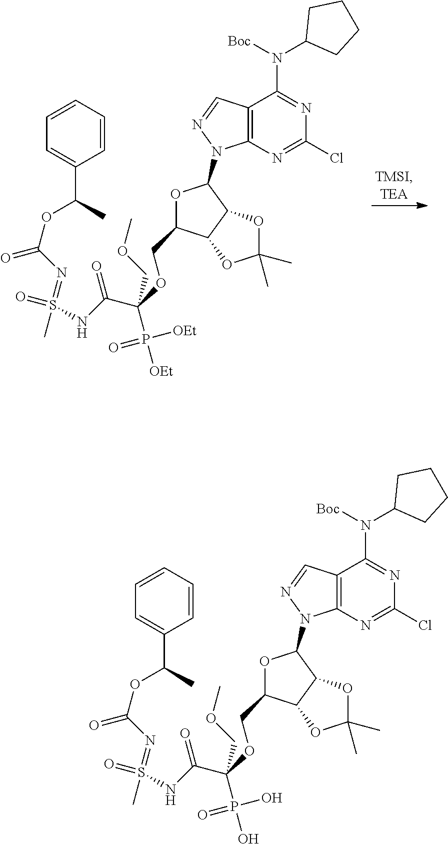 [SMILES: CCOP(=O)(OCC)[C@](COC)(OC[C@H]1O[C@@H](n2ncc3c(N(C(=O)OC(C)(C)C)C4CCCC4)nc(Cl)nc32)[C@@H]2OC(C)(C)O[C@@H]21)C(=O)N[S@](C)(=O)=NC(=O)O[C@H](C)c1ccccc1 ; COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N[S@](C)(=O)=NC(=O)O[C@H](C)c1ccccc1)P(=O)(O)O]

### Step 1

To a solution of tert-butyl (6-chloro-1-((3aR,4R,6R,6aR)-6-((((S)-2-(diethoxyphosphoryl)-3-methoxy-1-oxo-1-((R)—N′—(((R)-1-phenylethoxy)carbonyl)methylsulfonoamidimidamido)propan-2-yl)oxy)methyl)-2,2-dimethyltetrahydrofuro[3,4-d][1,3]dioxol-4-yl)-1H-pyrazolo[3,4-d]pyrimidin-4-yl)(cyclopentyl)carbamate (200 mg, 205.67 μmol, 1 eq) in DCM (3 mL) was added TMSI (617.29 mg, 3.09 mmol, 419.93 μL, 15 eq) and TEA (416.23 mg, 4.11 mmol, 572.53 μL, 20 eq) at 0° C. The mixture was stirred at 0° C. for 2 h. The reaction was poured into water (20 mL). The mixture was extracted with ethyl acetate (3×4 mL). The combined organic layers was washed with brine and dried over Na₂SO₄. The organic layer was concentrated under high vacuum. The residue was purified by prep-HPLC (column: Waters Xbridge C18 150*50 mm*10 um; mobile phase: [H₂O (0.05% NH₃H₂O+10 mM NH₄HCO₃)-ACN]; gradient: 30%-60% B over 8.0 min) to give ((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxo-1-((R)—N′—(((R)-1-phenylethoxy)carbonyl)methylsulfonoamidimidamido)propan-2-yl)phosphonic acid (60 mg, 65.48 μmol, 31.84% yield) as a white solid.

¹H NMR: 400 MHz, CDCl₃. δ=1.38 (s, 3H), 1.54 (s, 9H), 1.86-2.03 (m, 7H), 2.18-2.33 (m, 7H), 3.38 (br s, 3H), 3.49-3.56 (m, 3H), 3.95-4.07 (m, 4H), 4.46 (dt, J=4.10, 2.02 Hz, 1H), 5.00-5.06 (m, 1H), 5.09-5.16 (m, 1H), 5.29-5.35 (m, 1H), 5.79 (dt, J=6.85, 3.39 Hz, 1H), 6.58 (br d, J=3.00 Hz, 1H), 7.29-7.37 (m, 5H), 8.04-8.11 (m, 1H).

### Step 2

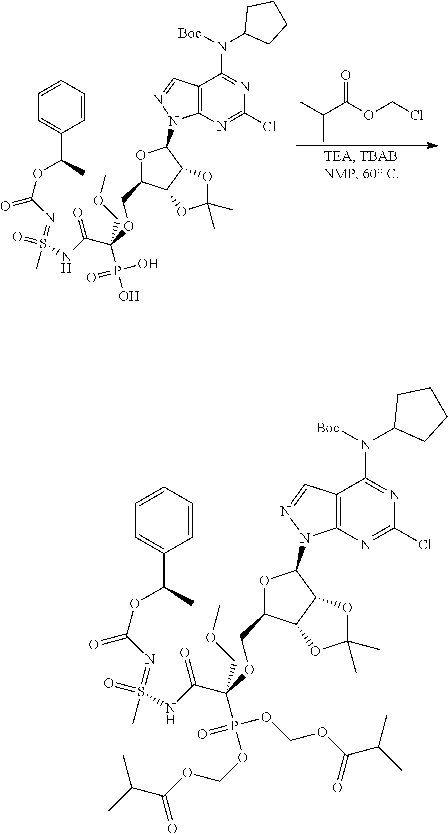

To a solution of ((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxo-1-((R)—N′—(((R)-1-phenylethoxy)carbonyl)methylsulfonoamidimidamido)propan-2-yl)phosphonic acid (50 mg, 54.57 μmol, 1 eq) in NMP (1 mL) was added TEA (143.56 mg, 1.42 mmol, 197.47 μL, 26 eq), TBAB (70.36 mg, 218.26 μmol, 4 eq) and chloromethyl 2-methylpropanoate (119.24 mg, 873.05 μmol, 16 eq). The mixture was stirred at 60° C. for 12 h. The reaction mixture was poured into water (10 mL). The mixture was extracted with ethyl acetate (3×2 mL). The combined organic layers was washed with brine and dried over Na₂SO₄. The organic layer was concentrated under high vacuum to give ((((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxo-1-((R)—N′—(((R)-1-phenylethoxy)carbonyl)methylsulfonoamidimidamido)propan-2-yl)phosphoryl)bis(oxy))bis(methylene)bis(2-methylpropanoate) (50 mg, 44.78 μmol, 82.07% yield) as a brown solid.

LCMS (ESI+): m/z 1116.3 [M+H]⁺, Rt:0.783 min.

### Step 3

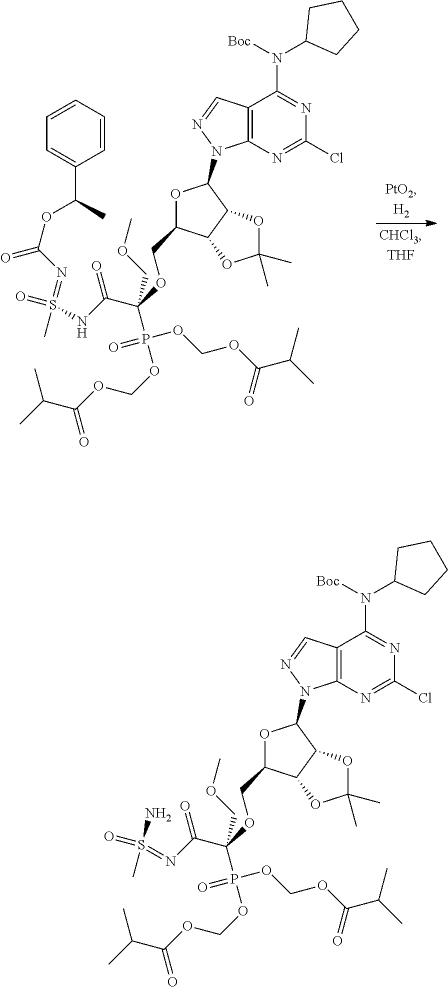 [SMILES: COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N[S@](C)(=O)=NC(=O)O[C@H](C)c1ccccc1)P(=O)(OCOC(=O)C(C)C)OCOC(=O)C(C)C ; COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N=[S@@](C)(N)=O)P(=O)(OCOC(=O)C(C)C)OCOC(=O)C(C)C]

To a mixture of PtO₂ (10.17 mg, 44.78 μmol, 1 eq) in EtOH (1 mL) was added ((((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxo-1-((R)—N′—(((R)-1-phenylethoxy)carbonyl)methylsulfonoamidimidamido)propan-2-yl)phosphoryl)bis(oxy))bis(methylene)bis(2-methylpropanoate) (50 mg, 44.78 μmol, 1 eq) and CHCl₃ (5.35 mg, 44.78 μmol, 3.60 μL, 1 eq) under H₂. The mixture was stirred at 25° C. for 2 h. The mixture was filtered. The filtrate was dried under high vacuum to give ((((2S)-1-((amino(methyl)(oxo)-λ⁶-sulfaneylidene)amino)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxopropan-2-yl)phosphoryl)bis(oxy))bis(methylene)bis(2-methylpropanoate) (50 mg, 30.98 μmol, 69.18% yield, 60% purity) as a brown solid.

LCMS (ESI+): m/z 968.4 [M+H]⁺, Rt:0.701 min.

### Step 4

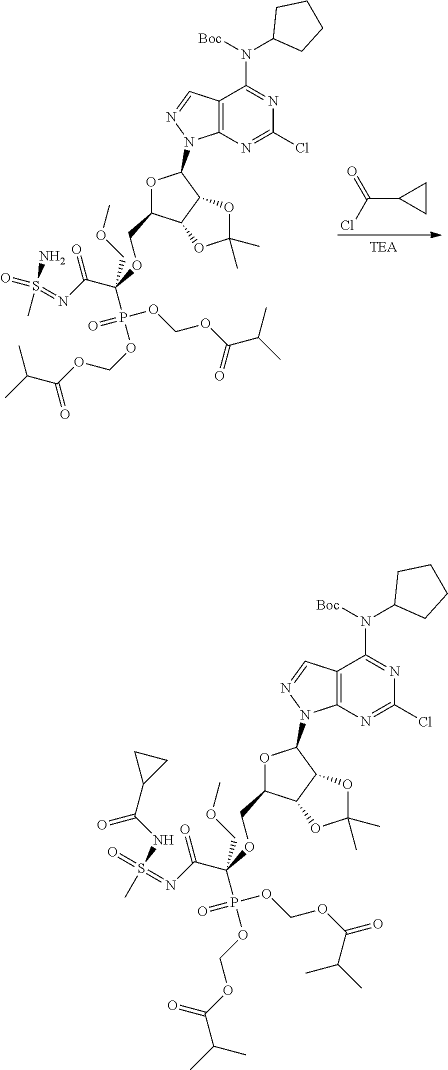 [SMILES: COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N=[S@@](C)(N)=O)P(=O)(OCOC(=O)C(C)C)OCOC(=O)C(C)C ; O=C(Cl)C1CC1 ; COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N=[S@](C)(=O)NC(=O)C1CC1)P(=O)(OCOC(=O)C(C)C)OCOC(=O)C(C)C]

To a solution of ((((2S)-1-((amino(methyl)(oxo)-λ⁶-sulfaneylidene)amino)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-3-methoxy-1-oxopropan-2-yl)phosphoryl)bis(oxy))bis(methylene)bis(2-methylpropanoate) (50 mg, 51.63 μmol, 1 eq) in DCM (1 mL) was added TEA (15.67 mg, 154.89 μmol, 21.56 μL, 3 eq) and cyclopropanecarbonyl chloride (10.79 mg, 103.26 μmol, 9.37 μL, 2 eq). The mixture was stirred at 25° C. for 2 h. The reaction mixture was poured into water (10 mL). The mixture was extracted with DCM (3×2 mL). The combined organic layers was washed with brine and dried over Na₂SO₄. The organic layer was concentrated under high vacuum. The residue was purified by prep-TLC (Ethyl acetate:Methanol=50:1) to give ((((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-1-(((S)-cyclopropanecarboxamido(methyl)(oxo)-λ⁶-sulfaneylidene)amino)-3-methoxy-1-oxopropan-2-yl)phosphoryl)bis(oxy))bis(methylene)bis(2-methylpropanoate) (30 mg, 28.94 μmol, 56.06% yield) as a white solid.

LCMS (ESI+): m/z 1036.4 [M+H]⁺, Rt: 0.756 min.

### Step 5

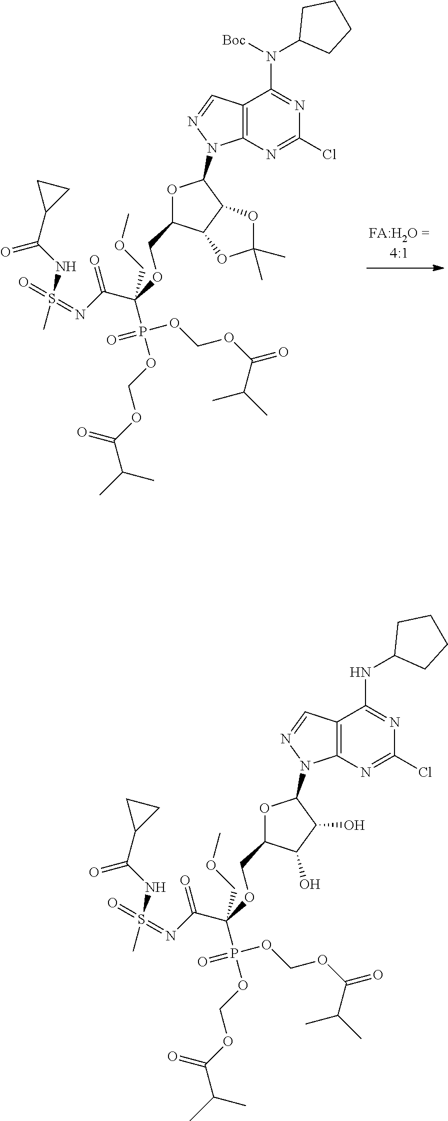 [SMILES: COC[C@@](OC[C@H]1O[C@@H](n2ncc3c(N(C(=O)OC(C)(C)C)C4CCCC4)nc(Cl)nc32)[C@@H]2OC(C)(C)O[C@@H]21)(C(=O)N=[S@](C)(=O)NC(=O)C1CC1)P(=O)(OCOC(=O)C(C)C)OCOC(=O)C(C)C ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(OCOC(=O)C(C)C)OCOC(=O)C(C)C]

To a solution of ((((S)-2-(((3aR,4R,6R,6aR)-6-(4-((tert-butoxycarbonyl)(cyclopentyl)amino)-6-chloro-1H-pyrazolo[3,4-d]pyrimidin-1-yl)-2,2-dimethyltetrahydrofuro[3,4-d][1,3]dioxol-4-yl)methoxy)-1-(((S)-cyclopropanecarboxamido(methyl)(oxo)-λ⁶-sulfaneylidene)amino)-3-methoxy-1-oxopropan-2-yl)phosphoryl)bis(oxy))bis(methylene)bis(2-methylpropanoate) (30 mg, 28.94 μmol, 1 eq) in H₂O (0.2 mL) was added HCOOH (1.39 mg, 28.94 μmol, 0.8 mL, 1 eq) at 0° C. The mixture was stirred at 25° C. for 12 h. The organic layer was separated and concentrated under high vacuum. The residue was purified by prep-HPLC (column: Waters Xbridge C18 150*50 mm*10 um; mobile phase: [H₂O (10 mM NH₄HCO₃)-ACN]; gradient: 25%-60% B over 8.0 min) to give the title compound (3 mg, 3.16 μmol, 10.93% yield, 94.5% purity) as a white solid.

¹H NMR: 400 MHz, MeOD-d₄. δ=0.73-0.81 (m, 2H), 0.89 (br s, 2H), 1.11-1.19 (m, 12H), 1.54-1.74 (m, 5H), 1.76-1.86 (m, 2H), 2.05-2.15 (m, 2H), 2.58 (quin, J=6.91 Hz, 2H), 3.35 (s, 3H), 3.42 (s, 3H), 3.82-3.89 (m, 1H), 3.90-3.96 (m, 1H), 3.98-4.06 (m, 2H), 4.22 (q, J=4.96 Hz, 1H), 4.50-4.58 (m, 1H), 4.70 (t, J=4.88 Hz, 1H), 4.79-4.82 (m, 1H), 5.61 (dd, J=13.07, 5.32 Hz, 1H), 5.65-5.71 (m, 3H), 6.17 (d, J=3.75 Hz, 1H), 8.10 (s, 1H).

LCMS (ESI+): m/z 895.9 [M+H]⁺, Rt: 1.950 min.

### Example 35

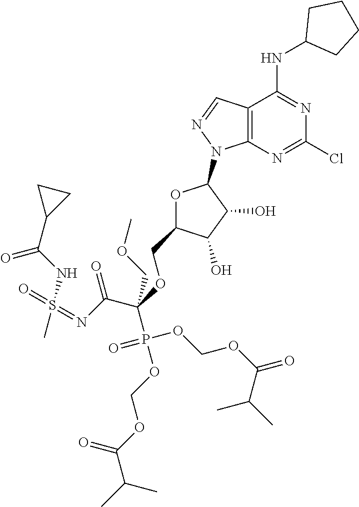 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(OCOC(=O)C(C)C)OCOC(=O)C(C)C]

### ((((S)-2-(((2R,3S,4R,5R)-5-(6-chloro-4-(cyclopentylamino)-1H-pyrazolo[3,4-d]pyrimidin-1-yl)-3,4-dihydroxytetrahydrofuran-2-yl)methoxy)-1-(((R)-cyclopropanecarboxamido(methyl)(oxo)-λ⁶-sulfaneylidene)amino)-3-methoxy-1-oxopropan-2-yl)phosphoryl)bis(oxy))bis(methylene)bis(2-methylpropanoate)

¹H NMR: 400 MHz, MeOD-d₄. δ=0.77-0.85 (m, 2H), 0.89-0.95 (m, 2H), 1.15-1.19 (m, 12H), 1.59-1.75 (m, 5H), 1.78-1.87 (m, 2H), 2.05-2.16 (m, 2H), 2.53-2.66 (m, 2H), 3.36 (br s, 3H), 3.45 (br s, 3H), 3.83-3.92 (m, 1H), 3.95-4.03 (m, 2H), 4.07 (dd, J=9.94, 3.94 Hz, 1H), 4.22 (br d, J=4.88 Hz, 1H), 4.53-4.59 (m, 1H), 4.74-4.78 (m, 1H), 4.82 (br s, 1H), 5.60-5.73 (m, 4H), 6.15-6.24 (m, 1H), 8.12 (s, 1H).

LCMS method: m/z 896.0 [M+H]⁺, Rt: 2.113 min.

### Biologic and Pharmacologic Assays and Methods

The activity of the compounds as CD73 inhibitors is illustrated in the following assay. The other compounds listed above which have not yet been made and/or tested are predicted to have activity in this assay as well.

### Biochemical CD73 Assay

Activity of recombinant CD73 was measured by quantification of free phosphate using the Malachite Green detection system (R&D Systems #DY996). Test compounds were solubilized in DMSO and dispensed into 384-well polystyrene plates in a 8-point 3× titration in duplicates. Then, 10 μL of 4 nM human CD73 enzyme (Novoprotein #C446) in phosphate-free assay buffer (10 mM HEPES, pH 7.4, 125 mM NaCl, 1 mM KCl, 10 mM glucose, 2 mM MgCl2) was added to the plates. The compound and enzyme were incubated for 15 min at RT. Then, 10 μL of 80 μM AMP (Sigma Aldrich #A1752) in the assay buffer was added and the reaction mixture was incubated for 45 min at 37° C. The final concentrations of CD73 and AMP in the reaction were 2 nM and 40 μM, respectively. The reaction was stopped by adding 5 μL of Malachite Green reagent A and incubating for 10 min at RT, followed by addition of 5 μL of Malachite Green reagent B and incubation for 20 min at RT. Absorbance was then read at 620 nM with the Flexstation spectrophotometer (Molecular Devices). The IC₅₀ values were determined using a four-parameter nonlinear regression curve fit.

### Cellular CD73 Assay

Primary human peripheral blood CD3+ T-cells obtained from AllCells were thawed and cultured for 24 h in a complete growth medium (Stemline T-cell expansion medium, Sigma #S1694) supplemented with 1 mM L-glutamine, 50 IU/mL of IL-2 and 25 μL/mL of Dynabeads™ Human T-Activator CD3/CD28 (Thermo-Fisher #11161D) at a density of 10⁶ cells/mL. Then, cells were labeled with 1.25 μM Cell Trace Violet (Thermo-Fisher #C34557) according to the manufacturer's instructions. Labeled cells were resuspended in a warm complete growth medium and plated onto U-bottom 96-well plates at 25,000 cells/90 μL/well. Next, 500× serial compound dilutions were prepared in DMSO in a 8-point 3-fold titration and then diluted 1:25 into a complete growth medium, generating 20× compound dilutions. Then, 5 μL of 20× compound dilutions was added to the cells and incubated for 60 min at 37° C. After this, 5 μL of 20×AMP was added to a final concentration of 40 μM. Treated cells were cultured for 3 days at 37° C., 5% CO₂. Thereafter, cells were gently pelleted by centrifugation, and supernatants were collected and frozen at −80° C. for subsequent cytokine analysis. Cell pellets were resuspended in 100 μL of PBS/2% FBS and gently pipetted up and down to produce a single cell suspension. Then, 3 μL of 7-AAD (BD Biosciences #559925) was added to each well and plates were incubated on ice for 5 min. Cells were then washed and resuspended in 150 μL of PBS/2% FBS. Divided and non-divided live cells were quantified by Attune NxT flow cytometer. Cell Trace Violet-labeled cells cultured without a T-cell activator were used as the nondividing control.

For IL-2 and IFN-γ cytokine analysis, supernatants were thawed on ice, diluted 1:10 in the buffer. The concentration of cytokines was determined using Meso Scale Discovery according to the manufacturer's instructions (Meso Scale Discovery). The EC₅₀ values were determined using a four-parameter nonlinear regression curve fit (GraphPad Prism Version 9.2, GraphPad Software Inc., San Diego, CA).

TABLE 2
Biological Activity

| Example # | CD73 IC₅₀ (nM) | CD73 EC₅₀ (nM) |
|---|---|---|
| 1 | 0.94 | 0.51 |
| 2 | 19 | — |
| 3 | 1 | 1.5 |
| 4 | 275 | — |
| 5 | 1.6 | — |
| 6 | 220 | — |
| 7 | 1.7 | — |
| 8 | 879 | — |
| 9 | 0.9 | 1.57 |
| 10 | 0.31 | 0.72 |
| 11 | 0.77 | 0.9 |
| 12 | 0.45 | 0.92 |
| 13 | 0.7 | 0.68 |
| 14 | 0.74 | 1.15 |
| 15 | 6.3 | 7.78 |
| 16 | 0.78 | 1.34 |
| 17 | 0.58 | 0.98 |
| 18 | 0.5 | 1 |
| 19 | — | — |
| 20 | 220 | — |
| 21 | 5.1 | — |
| 22 | 0.21 | — |
| 23 | 0.47 | — |
| 24 | 2.3 | — |
| 25 | 1.13 | — |
| 26 | 2.36 | — |
| 27 | 7.8 | — |
| 28 | 1 | — |
| 29 | 10 | — |
| 30 | 0.6 | — |
| 31 | 0.5 | — |
| 32 | 1 | — |
| 33 | 11 | — |
| 34 | — | — |
| 35 | — | — |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

1. A compound of Formula I: 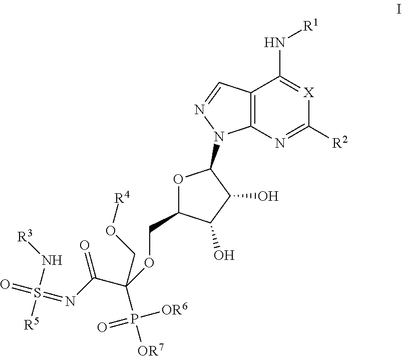
or a pharmaceutically acceptable salt thereof, wherein:
X is chosen from CH and N;
R¹ is chosen from C₁-C₆ alkyl, C₃-C₁₂ cycloalkyl, heterocycloalkyl, aryl, C₁-C₆ alkylaryl, C₁-C₆ alkylheteroaryl, C₃-C₅ cycloalkylaryl, C₃-C₅ cycloalkylheteroaryl, and heteroaryl, each of which is optionally substituted with one or more groups chosen from C₁-C₆ alkyl, C₁-C₆ alkoxy, C₁-C₆ fluoroalkyl, C₁-C₄ fluoroalkoxy, halo, and hydroxy;
R² is chosen from hydrogen, halo, aryl, cyano, C₃-C₅ cycloalkyl, and heteroaryl;
R³ is chosen from hydrogen, C₁-C₆ alkyl, —C(O)R⁸, —C(O)OR⁸, and —C(O)NHR⁸;
R⁴ is chosen from hydrogen, C₃-C₅ cycloalkyl, and C₁-C₆ alkyl;
R⁵ is C₁-C₆ alkyl;
R⁶ and R⁷ are each independently chosen from hydrogen, C₁-C₆ alkyl, —C(O)R⁹, and C₁-C₆ alkylcarboxylmethyl;
R⁸ is chosen from C₁-C₆ alkyl, C₃-C₇ cycloalkyl, 3- to 7-membered heterocycloalkyl, heteroaryl, and aryl, any of which may be substituted by one or more groups chosen from C₁-C₃ alkyl and halo; and
R⁹ is C₁-C₇ alkyl.

2. The compound of claim 1, or a pharmaceutically acceptable salt thereof, wherein X is N.

3. The compound of claim 1, or a pharmaceutically acceptable salt thereof, wherein X is CH.

4. The compound of claim 1, or a pharmaceutically acceptable salt thereof, wherein R¹ is cyclopentyl or cyclohexyl, each of which is optionally substituted with C₁-C₆ alkyl, C₁-C₆ alkoxy, C₁-C₆ fluoroalkyl, C₁-C₄ fluoroalkoxy, halo, and hydroxy.

5. The compound of claim 4, or a pharmaceutically acceptable salt thereof, wherein R¹ is cyclopentyl or cyclohexyl.

6. The compound of claim 5, or a pharmaceutically acceptable salt thereof, wherein R¹ is cyclopentyl.

7. The compound of claim 1, or a pharmaceutically acceptable salt thereof, wherein R² is halo.

8. The compound of claim 7, or a pharmaceutically acceptable salt thereof, wherein R² is chloro.

9. The compound of claim 1, or a pharmaceutically acceptable salt thereof, wherein R² is 1H-1,2,3-triazol-1-yl.

10. The compound of claim 1, or a pharmaceutically acceptable salt thereof, wherein X is N and R² is halo.

11. The compound of claim 10, or a pharmaceutically acceptable salt thereof, wherein R² is chloro.

12. The compound of claim 1, or a pharmaceutically acceptable salt thereof, wherein the compound of Formula I is a compound of Formula II, 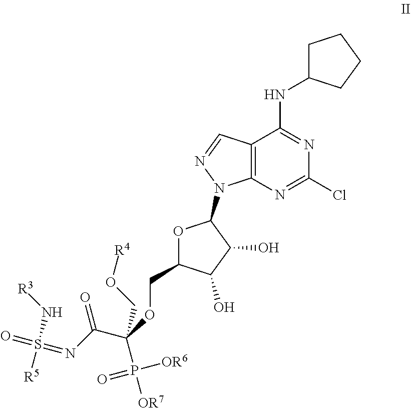
or a pharmaceutically acceptable salt thereof.

13. The compound of claim 1, or a pharmaceutically acceptable salt thereof, wherein the compound of Formula I is a compound of Formula III, 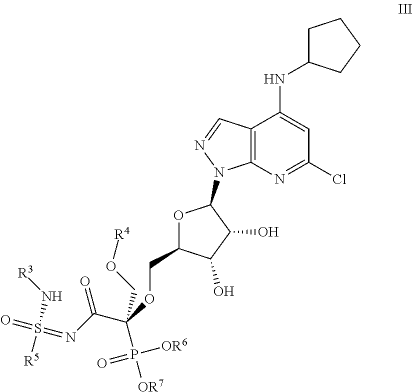
or a pharmaceutically acceptable salt thereof.

14. The compound of claim 1, or a pharmaceutically acceptable salt thereof, wherein R⁴ is C₁-C₆ alkyl.

15. The compound of claim 14, or a pharmaceutically acceptable salt thereof, wherein R⁴ is methyl.

16. The compound of claim 1, or a pharmaceutically acceptable salt thereof, wherein R⁶ is hydrogen.

17. The compound of claim 1, or a pharmaceutically acceptable salt thereof, wherein R⁷ is hydrogen.

18. The compound of claim 1, or a pharmaceutically acceptable salt thereof, wherein R⁶ and R⁷ are each hydrogen.

19. The compound of claim 1, or a pharmaceutically acceptable salt thereof, wherein R⁶ and R⁷ are each —CH₂OC(O)CH(CH₃)₂.

20. The compound of claim 1, or a pharmaceutically acceptable salt thereof, wherein R⁵ is methyl.

21. The compound of claim 1, or a pharmaceutically acceptable salt thereof, wherein R³ is chosen from hydrogen, —C(O)R⁸, —C(O)OR⁸, and —C(O)NHR⁸.

22. The compound of claim 21, or a pharmaceutically acceptable salt thereof, wherein R³ is hydrogen.

23. The compound of claim 21, or a pharmaceutically acceptable salt thereof, wherein R³ is —C(O)R⁸ and R⁸ is chosen from methyl, cyclopropyl, ethyl, isopropyl, oxetanyl, methylpyrazolyl, pyridinyl, and fluorophenyl.

24. The compound of claim 20, or a pharmaceutically acceptable salt thereof, wherein R³ is chosen from —C(O)OR⁸ or —C(O)NHR⁸ and R⁸ is C₁-C₃ alkyl.

25. The compound of claim 24, or a pharmaceutically acceptable salt thereof, wherein R⁸ is methyl.

26. The compound of claim 1, or a pharmaceutically acceptable salt thereof, wherein the compound of Formula I is chosen from

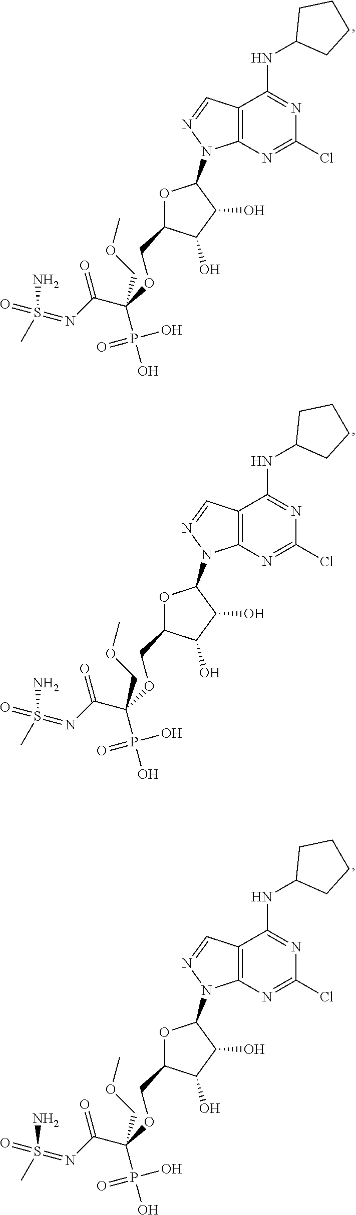 [SMILES: COC[C@@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O ; COC[C@](OC[C@H]1O[C@@H](n2ncc3c(NC4CCCC4)nc(Cl)nc32)[C@H](O)[C@@H]1O)(C(=O)N=[S@](C)(N)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O]

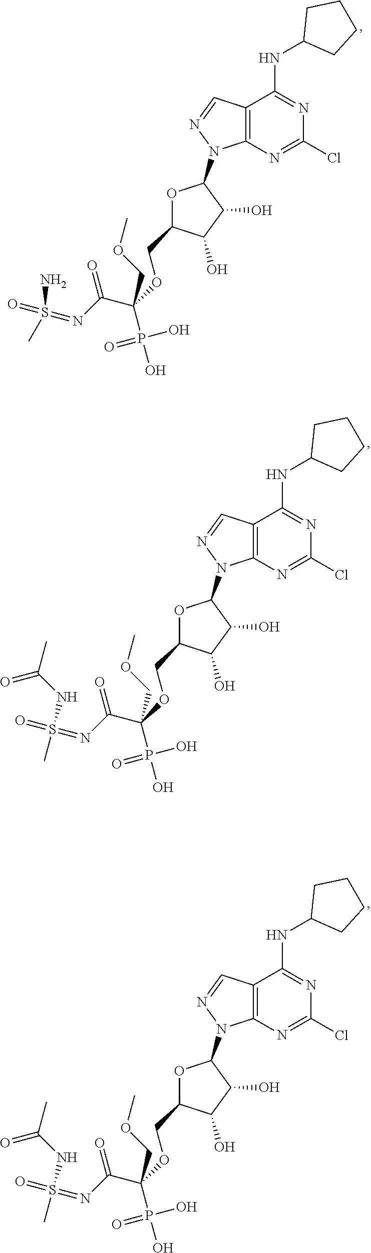 [SMILES: COC[C@](OC[C@H]1O[C@@H](n2ncc3c(NC4CCCC4)nc(Cl)nc32)[C@H](O)[C@@H]1O)(C(=O)N=[S@@](C)(N)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O]

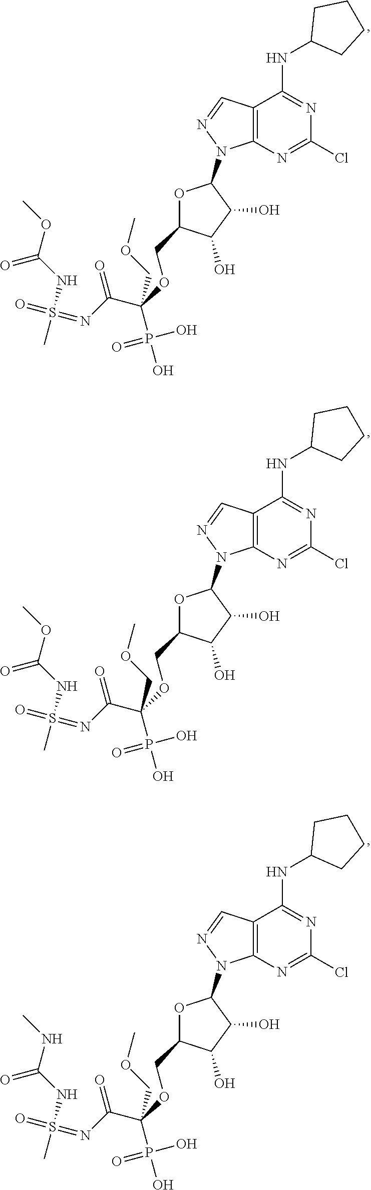 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(=O)OC)P(=O)(O)O ; CNC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O]

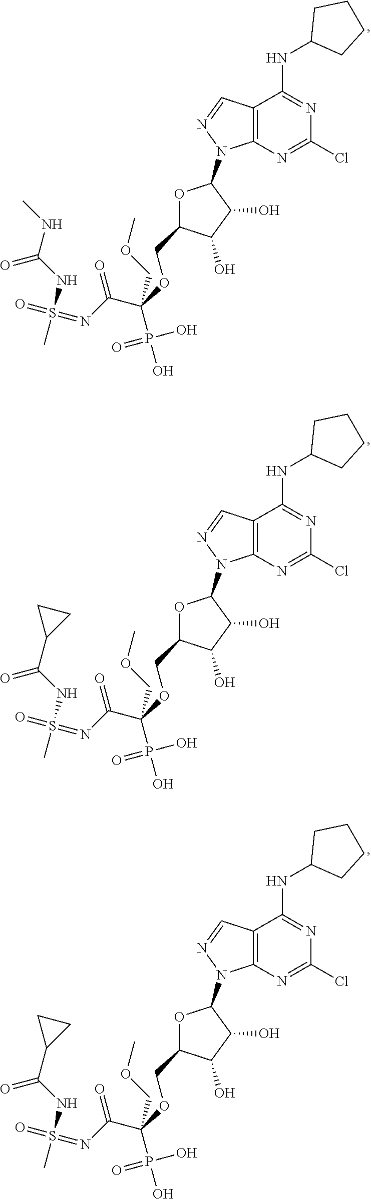 [SMILES: CNC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(O)O]

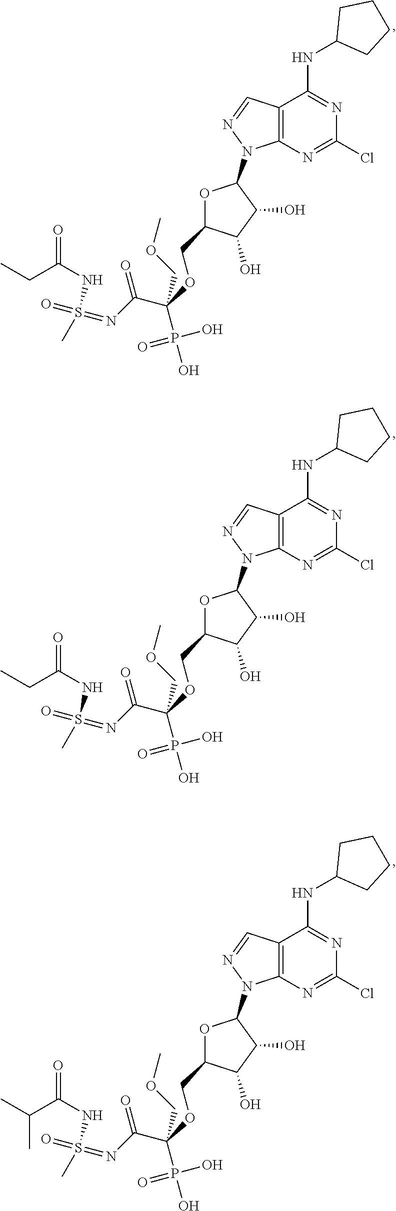 [SMILES: CCC(=O)N[S@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; CCC(=O)N[S@@](C)(=O)=NC(=O)[C@@](COC)(OC[C@H]1O[C@@H](n2ncc3c(NC4CCCC4)nc(Cl)nc32)[C@H](O)[C@@H]1O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C(C)C)P(=O)(O)O]

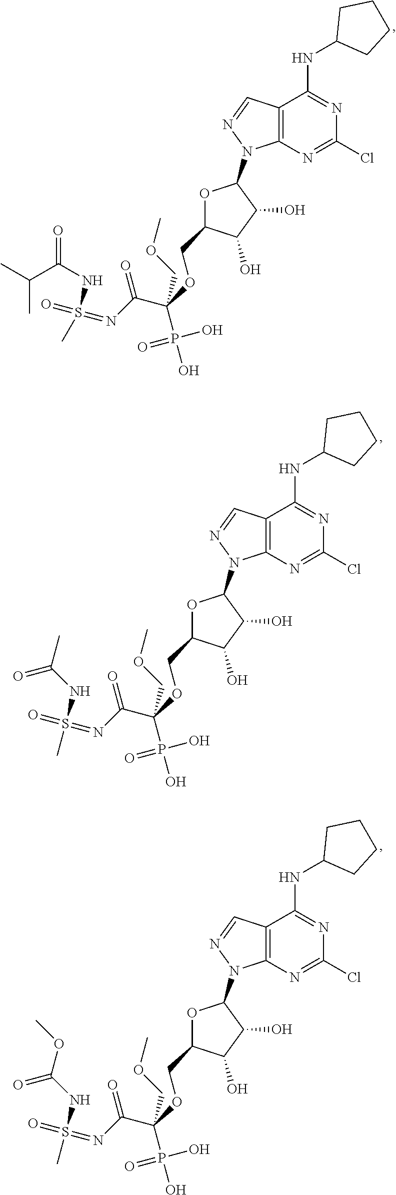 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C(C)C)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(C)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)OC)P(=O)(O)O]

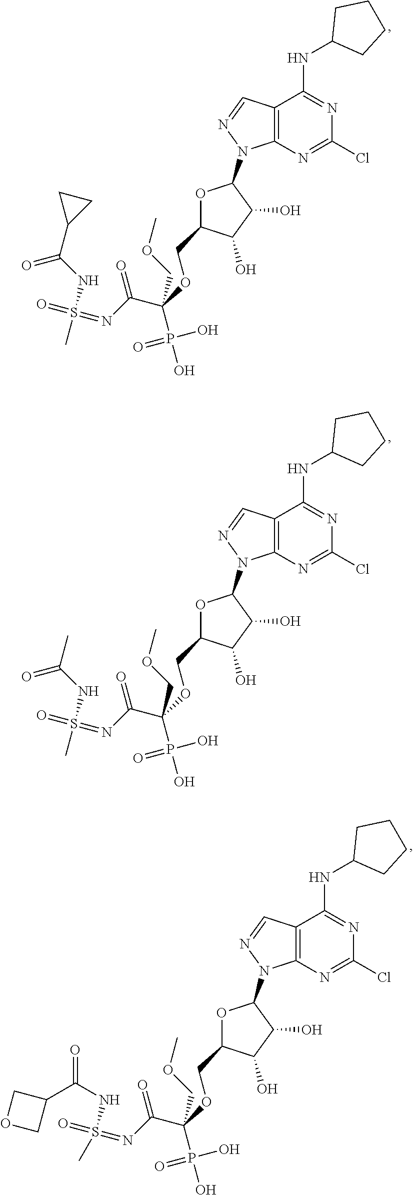 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(O)O ; COC[C@](OC[C@H]1O[C@@H](n2ncc3c(NC4CCCC4)nc(Cl)nc32)[C@H](O)[C@@H]1O)(C(=O)N=[S@@](C)(=O)NC(C)=O)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1COC1)P(=O)(O)O]

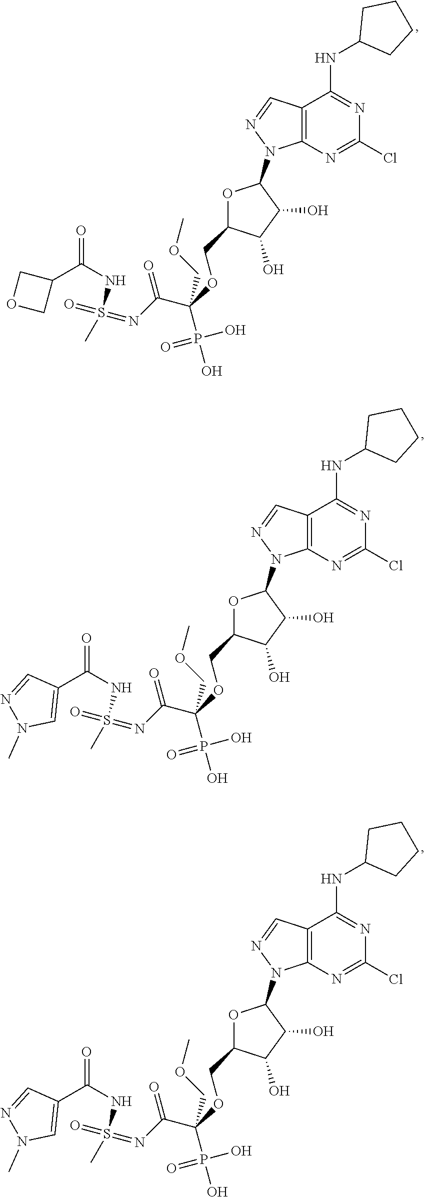 [SMILES: COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1COC1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cnn(C)c1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1cnn(C)c1)P(=O)(O)O]

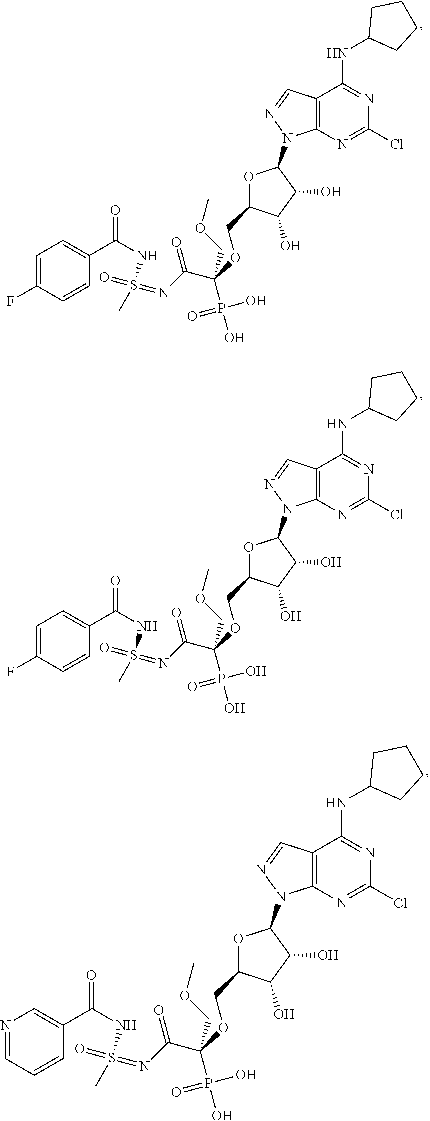 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1ccc(F)cc1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)c1ccc(F)cc1)P(=O)(O)O ; COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)c1cccnc1)P(=O)(O)O]

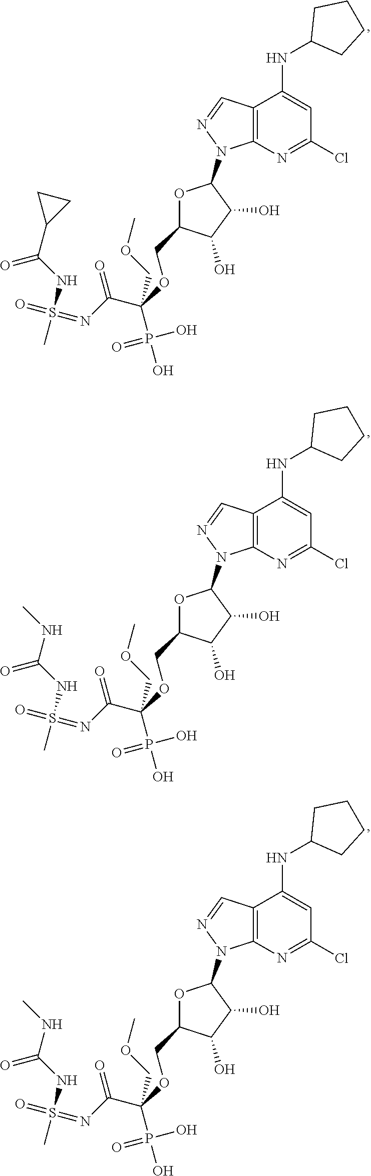 [SMILES: COC[C@@](OC[C@H]1O[C@@H](n2ncc3c(NC4CCCC4)cc(Cl)nc32)[C@H](O)[C@@H]1O)(C(=O)N=[S@](C)(=O)NC(=O)C1CC1)P(=O)(O)O ; CNC(=O)N[S@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O ; CNC(=O)N[S@@](C)(=O)=NC(=O)[C@@](COC)(OC[C@H]1O[C@@H](n2ncc3c(NC4CCCC4)cc(Cl)nc32)[C@H](O)[C@@H]1O)P(=O)(O)O]

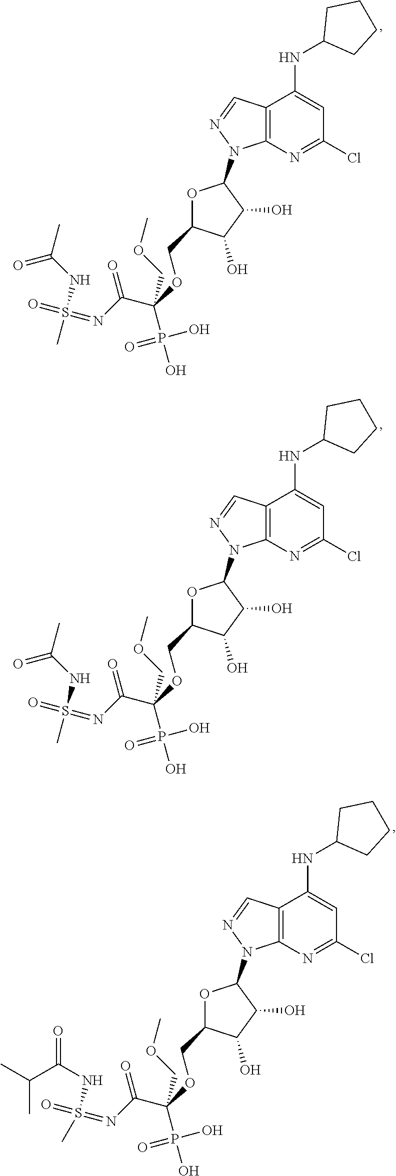 [SMILES: COC[C@@](OC[C@H]1O[C@@H](n2ncc3c(NC4CCCC4)cc(Cl)nc32)[C@H](O)[C@@H]1O)(C(=O)N=[S@@](C)(=O)NC(C)=O)P(=O)(O)O ; COC[C@@](OC[C@H]1O[C@@H](n2ncc3c(NC4CCCC4)cc(Cl)nc32)[C@H](O)[C@@H]1O)(C(=O)N=[S@](C)(=O)NC(C)=O)P(=O)(O)O ; COC[C@@](OC[C@H]1O[C@@H](n2ncc3c(NC4CCCC4)cc(Cl)nc32)[C@H](O)[C@@H]1O)(C(=O)N=[S@@](C)(=O)NC(=O)C(C)C)P(=O)(O)O]

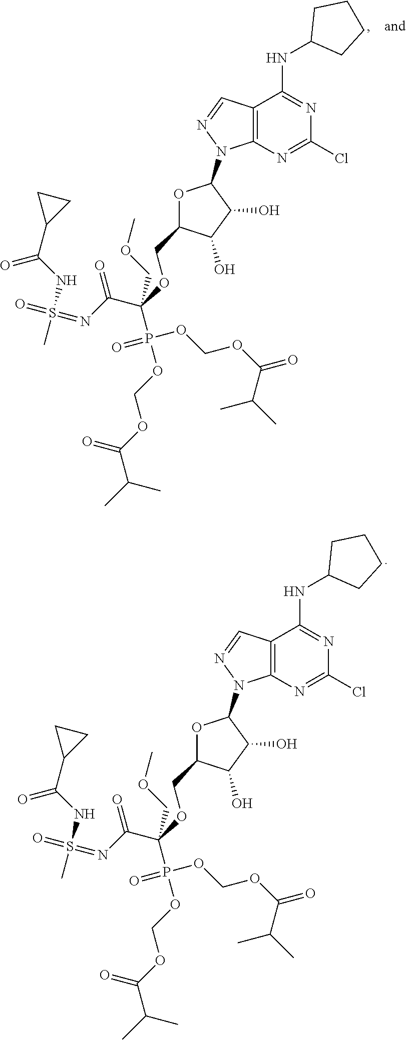 [SMILES: COC[C@@](OC[C@H]1O[C@@H](n2ncc3c(NC4CCCC4)nc(Cl)nc32)[C@H](O)[C@@H]1O)(C(=O)N=[S@@](C)(=O)NC(=O)C1CC1)P(=O)(OCOC(=O)C(C)C)OCOC(=O)C(C)C ; COC[C@@](OC[C@H]1O[C@@H](n2ncc3c(NC4CCCC4)nc(Cl)nc32)[C@H](O)[C@@H]1O)(C(=O)N=[S@](C)(=O)NC(=O)C1CC1)P(=O)(OCOC(=O)C(C)C)OCOC(=O)C(C)C]

27. A pharmaceutical composition comprising the compound of claim 1, or a pharmaceutically acceptable salt thereof, and a pharmaceutically acceptable carrier.

28. The pharmaceutical composition of claim 27, further comprising at least one additional pharmaceutically active agent, wherein the additional pharmaceutically active agent is a chemotherapeutic agent.

29. (canceled)

30. A method of treatment of a CD73-mediated disease, comprising the administration of a therapeutically effective amount of a compound as recited in claim 1, or a pharmaceutically acceptable salt thereof, to a patient having a CD73-mediated disease.

31. The method of claim 30, wherein the CD73-mediated disease is cancer.

32. The method of claim 31, wherein the cancer is breast cancer.

33. The method of claim 32, wherein the breast cancer is triple-negative breast cancer.

34. The method of claim 31, wherein the cancer is chosen from melanoma, renal cell carcinoma, colorectal carcinoma, pancreatic cancer, prostate cancer, ovarian cancer, gastric cancer, leukemia and lymphoma.

35. The method of claim 30, wherein the method further comprises administering one or more additional pharmaceutically active agents.

36. The method of claim 35, wherein the additional pharmaceutically active agent is a chemotherapeutic agent.
